(19) United States
(12) Patent Application Publication
(10) Pub. No.: US 2014/0135310 A1
(43) Pub. Date: May 15, 2014

(54) SULPHONAMIDE DERIVATIVES OF ALICYCLIC AMINES FOR THE TREATMENT OF CENTRAL NERVOUS SYSTEM DISEASES

(71) Applicants: Marcin Kolaczkowski, Wieliczka (PL); Monika Marcinkowska, Krakow (PL); Adam Bucki, Proszowice (PL); Maciej Pawlowski, Wieliczka (PL); Andrzej Krukowski, Warszawa (PL); Rafal Rusiecki, Warszawa (PL); Agata Magdalena Siwek, Brzeszcze (PL); Malgorzata Anna Wolak, Krakow (PL)

(72) Inventors: Marcin Kolaczkowski, Wieliczka (PL); Monika Marcinkowska, Krakow (PL); Adam Bucki, Proszowice (PL); Maciej Pawlowski, Wieliczka (PL); Andrzej Krukowski, Warszawa (PL); Rafal Rusiecki, Warszawa (PL); Agata Magdalena Siwek, Brzeszcze (PL); Malgorzata Anna Wolak, Krakow (PL)

(73) Assignee: Adamed Sp. z o.o., Czosnów k/Warszawy (PL)

(21) Appl. No.: 14/127,435

(22) PCT Filed: Jun. 29, 2012
(86) PCT No.: PCT/IB2012/053318

§ 371 Date: Dec. 18, 2013

(30) Foreign Application Priority Data
Jun. 29, 2011 (PL) P-395470

(51) Int. Cl. C07D 417/12; C07D 413/14; C07D 405/12; C07D 401/12; C07D 409/14; C07D 413/06; C07D 403/12

(52) U.S. Cl. CPC C07D 417/12; C07D 413/06; C07D 413/14; C07D 403/12; C07D 401/12; C07D 409/14; C07D 405/12
USPC 514/210.21; 548/241; 546/198; 548/467; 546/201; 548/953; 548/526; 546/197; 514/379; 514/321; 514/414; 514/323; 514/210.19; 514/422

(57) ABSTRACT

Sulphonamide derivatives of alicyclic amines of formula (I), wherein A represents naphthyl or 9- or 10-membered bicyclic group, consisting of benzene ring fused with 5- or 6-membered heterocyclic ring; D represents phenyl, naphthyl, 5-membered aromatic heterocyclic group, bicyclic group consisting of a ring selected from benzene and pyridine, fused with aromatic or non-aromatic 5-membered heterocyclic ring; p, r independently represent 0 or 1; x, z independently represent 1 or 2; n is 2 or 3;

and enancjomers, pharmaceutically acceptable salts and solvates thereof.

The compounds may be useful for the treatment and/or prevention of the central nervous system disorders.

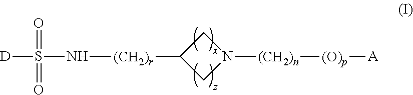

### FIELD OF THE INVENTION

The present invention relates to novel sulphonamide derivatives of alicyclic amines having affinity to dopaminergic, serotoninergic, adrenergic receptors and to serotonin transporter receptors, pharmaceutical compositions containing the same and to the use thereof. The compounds may be useful for the treatment of diseases of the central nervous system (CNS), such as schizophrenia, bipolar affective disorder, depression, anxiety disorders, sleep disorders or Alzheimer disease.

### STATE OF ART

CNS disorders are considered a global medical problem. A number of people suffering from those diseases constantly grows, particularly in highly developed countries and intensively developing ones.

Among all psychiatric diseases, schizophrenia, depression, bipolar affective disorder, anxiety, sleep disorders and addictions are the major ones. The main neurologic disorders are Alzheimer's disease, Parkinson's disease, epilepsy and different pain disorders.

Antipsychotic drugs, which are main treatment of schizophrenia, are divided into two main classes on the basis of their liability to induce neurological side effects after long-term treatment. Typical antipsychotic drugs, such as chlorpromazine and haloperidol, induce after repeated administration various extrapyramidal side effects (EPS) including Parkinson-like symptoms and tardive dyskinesia. Repeated treatment with so called atypical antipsychotic drugs, such as clozapine, risperidone, olanzapine, quetiapine, ziprasidone and aripiprazole, is associated with a lower incidence of neurological side effects. Typical antipsychotics reduce positive symptoms but do not reduce negative symptoms and cognitive dysfunctions. Plasma prolactin levels are increased in humans, and there is a gain in body weight potentially leading to the development of metabolic syndrome. Atypical antipsychotic drugs effectively reduce positive symptoms and also to some extent negative symptoms and cognitive disturbances, while producing less serious EPS. Atypical antipsychotic drugs differ in their propensity to elevate plasma prolactin levels in humans. Typical antipsychotic drugs block dopamine D2 receptors in the mesolimbic and nigrostriatal system. This mechanism is responsible for the antipsychotic effect (reduction of positive symptoms) as well as induction of EPS. Clinical support for the dopamine hypothesis of antipsychotic drug action was provided by PET findings of high dopamine D2 receptor occupancy in the striatum of patients responding to different antipsychotic drug treatments. Patients with a good response show dopamine D2 receptor occupancy of more than 65% (Nord M, Farde L., CNS Neuroscience & Therapeutics. 2010; 17:97.). The occurrence of EPS seems to be related to a higher occupancy of dopamine D2 receptors (above 80%). Atypical antipsychotics, also called second generation antipsychotic drugs, have clinical approvals for the treatment of psychosis and mania. Each drug has a unique pharmacodynamic and pharmacokinetic profile. Some of atypical antipsychotic drugs have additional antidepressant, anxiolytic or hypnotic profile (Schwartz T. L., Stahl S. M., CNS Neurosci. Ther.; 17(2), 110-7, 2011). Atypical antipsychotic drugs have in common a potent serotonin 5-HT2A receptor antagonism in relation to a weaker dopamine D2 receptor antagonism. This pharmacodynamic property is the basis of “atypicality” (Meltzer H. Y., Neuropsychopharmacology; 1, 193-6, 1989). Antagonism of 5-HT2A receptors likely allows more dopamine activity and neurotransmission to occur in the nigrostriatal system to avoid EPS. The same mechanism may allow small improvement in negative symptoms, and 5-HT2 antagonism in the tuberoinfundibular pathway may help to avoid hyperprolactinemia (Schwartz T. L., Stahl S. M., CNS Neurosci. Ther.; 17(2), 110-7, 2011).

Dopaminergic D2 receptors are the primary biological target of antipsychotic therapy. It is a recognized fact that blockade of these receptors in the mesolimbic system is responsible for the antipsychotic activity of neuroleptics, in particular for preventing positive symptoms. All antipsychotic drugs currently used exhibit at least moderate affinity for dopamine D2 receptors. However, blockade of these receptors in the nigrostriatal system if not compensated by a partial agonism to these receptors or by affecting other receptors (5-HT2A, 5-HT1A, alfa2c), may be a cause of extrapyramidal disorders, such as drug-induced parkinsonism, and within tuberoinfundibular pathway—of hyperprolactinaemia (Miyamoto S. et al., Mol. Psychiatry; 10(1), 79-104, 2005).

Dopaminergic D3 receptors are localized in limbic cortex and thus a preferential blockade of these receptors offers locally selective antidopaminergic activity. This results in increased effectiveness in reducing positive symptoms of schizophrenia sparing the blockade of extrapyramidal system and therefore reduces the risk of the main side effect such as pseudoparkinson's syndrome. Moreover, several preclinical data suggests that D3 dopamine receptor antagonism is more efficient in reducing the negative symptoms of schizophrenia and improves working memory. (Gray, J. A., Roth B. L.; Schizophr. Bull.; 33(5, 1100-19, 2007).

Serotoninergic neurons interact with dopaminergic neurons. Antagonistic activity of antipsychotics against serotoninergic receptors 5-HT2A type can stimulate the release of dopamine in the extrapyramidal, tuberoinfundibular systems and prefrontal cortex but not in the limbic system, what can result in alleviation of undesirable extrapyramidal symptoms and hyperprolactinaemia induced by D2 receptor blockade and in increased effectiveness of the drug against some of negative symptoms of schizophrenia, without increasing the positive symptoms. It is considered that high affinity for 5-HT2A receptors, higher than for D2 receptors, is one of the reasons of atypicality of the second-generation antipsychotics. Similar effects to those caused by the blockade of 5-HT2A receptors, are achieved by stimulation of serotonin receptor type 5-HT1A (aripiprazole, ziprasidone). It is assumed that stimulation of 5-HT1A receptors takes part in the antipsychotic effect in combination with D2 receptor blockade, especially in the safety profile of drug as well as is beneficial in fighting mood and cognitive symptoms of schizophrenia (Kim D. et al., Neurotheropeutics, 6(1), 78-85, 2009).

Serotoninergic receptors type 5-HT6 are almost exclusively localized in the central nervous system (CNS). Both the localization of the 5-HT6 receptors in limbic and cortical brain areas and relatively potent affinity and antagonistic activity of several antipsychotics (clozapine, olanzapine, sertindole) and antidepressants (mianserin, amitryptiline) at 5-HT6 receptors are suggestive of a potential role in pathophysiology and treatment of CNS disorders. Recent data in the literature indicate that blockade of 5-HT6 receptors may be implicated in a pro-cognitive effect due to the increase in cholinergic transmission, in antidepressant activity due to the increase in noradrenergic and dopaminergic one, as well as in an anxiolytic effect, It is evident that 5-HT6 receptor has emerged as a very interesting molecular target and antagonists of this receptor may serve as potential drugs in treatment of disorders characterized by cognitive impairments, such as Alzheimer's disease, schizophrenia, depression, anxiety (Liu K., Robichaud A., Drug Development Research 70,145-168, 2009; Wesotowska, A; Nikiforuk, A, Neuropharmacology 52(5), 1274-83, 2007). Moreover, 5-HT6 receptor antagonists have been demonstrated to be active in reduction of food intake and body weight by clinically approved mechanism that is consistent with the enhancement of satiety. Hence, several compounds with 5-HT6 receptor antagonistic activity are currently being clinically evaluated for the treatment of obesity (Heal D. et al., Pharmacology therapeutics, 117(2), 207-231, 2008).

Intensive research conducted since 1993 indicates that serotoninergic 5-HT7 receptors may play some role in the control of circadian rhythms, sleep, thermoregulation, cognitive processes, pain and migraine, as well as in neuronal excitability. Potent affinity and antagonistic activity of several antipsychotic and antidepressant drugs at 5-HT7 receptors suggest a potential role of these receptors in pathophysiology of many neuropsychiatric disorders. Taking into account the behavioral data presented in the literature, it has been established that selective 5-HT7 receptor antagonists produce antidepressant and anxiolytic activity in rats and mice (Wesotowska A. et al., Neuropharmacology 51, 578-586, 2006). Using mouse models of antipsychotic activity, Galici et al. showed that a selective 5-HT7 receptor antagonist SB-269970 may also evoke antipsychotic-like effects (Galici R. et al., Behav. Pharmacol.; 19(2), 153-9, 2008).

Serotoninergic 5-HT2C and histaminergic H1 receptors localized in hypothalamus play important role in food intake regulation. Blockade of both types of these receptors produced by antipsychotic drugs is most closely correlated with increased risk of weight gain and diabetes. On the other hand, blockade of 5-HT2C receptors, mostly localized in cortical areas and in the hippocampus, striatum, septal nuclei, thalamic and midbrain nuclei, may produce beneficial antidepressant and pro-cognitive effects. In the substantia nigra, 5-HT2C receptors are co-localised with GABA, indicating that they yield indirect control of dopaminergic transmission. Consequently, the blockade of 5-HT2C receptors, together with the 5-HT2A receptor one, would potentiate the D2 receptor-mediated tonic inhibitory control of dopaminergic projection, with protective effect against extrapyramidal symptoms (Kim D. et al., Neurotherapeutics, 6(1), 78-85, 2009). Histaminergic H1 receptor blockade produced by antipsychotic drugs may be implicated in sedative effect that is clinically profitable in controlling arousal that accompanies the acute phase of psychosis. It seems that simultaneous reduction in affinity of new molecule for both types of these receptors may be an element that protects against excessive body weight. However, the total elimination of affinity for these receptors may not be necessary because of certain benefits of blockade of 5-HT2C and H1 receptors.

Blockade of alpha2 adrenergic receptors potentiates antidepressants-induced increase of extracellular monoamines. This may suggest that substances inhibiting monoamine transporters and simultaneously blocking alpha2 adrenergic receptors may be potent and fast acting new antidepressants. Moreover, alpha2 antagonists potentiate acetylcholine secretion in the frontal cortex and may improve cognitive functions, what may provide additional advantages both in antidepressant therapy and antipsychotic therapy (especially improvement in negative symptoms). Blockade of alpha2 adrenergic receptors may also counteract sexual dysfunctions caused by serotonin reuptake inhibitors (Millan M., Neurotherapeutics, 6(1), 53-77, 2009). Alpha2 antagonists may also be beneficial in reducing extrapyramidal symptoms caused by blockade of D2 receptors in the striatum. Similarly, blockade of alpha1 adrenergic receptors, despite potential peripheral adverse effects involving hypotension, may cause some central nervous system benefits involving decrease in the risk of extrapyramidal side effects caused be antipsychotics. This may be associated with interaction between noradrenergic and serotoninergic neurons (Horacek J. et al., CNS Drugs, 20(5), 389-409, 2006).

Sigma receptors are a separate group of CNS receptors; however their physiological role is still unknown. It has been shown that some psychotomimetic substances like phencyclidine, metamphetamine, heroin or dextrometorphan are potent sigma receptor agonists. On the other hand, a classic antipsychotic drug haloperidol is a strong antagonist of sigma receptors, what may be important for its antipsychotic potential. It has been established that selective sigma receptor agonists may produce antidepressant effect (Cobos E. et al., Curr. Neuropharmacol., 6(4), 344-66, 2008). The above findings provide evidence that sigma receptors affinity may contribute to the overall beneficial pharmacological profile of a new psychotropic drug.

Because of important role of cholinergic system in the cognitive processes, current research is focused on substances which can directly or indirectly potentiate the activity of cholinergic system. This includes substances which are agonists of selected subtypes of nicotinic or muscarinic receptors and antagonists of 5-HT6 receptors. On the other hand, potential procognitive effects evoked by interaction with the above receptors may be masked by cholinolytic activity. Thus, in the scope of interest are substances free of antagonistic properties against cholinergic receptors. Moreover, this strategy allows to eliminate many undesired peripheral autonomic effects like constipations, dry mouth or tachycardia (Miyamoto S. et al., Mol. Psychiatry; 10(1), 79-104, 2005). In addition, it has been found that M3 muscarinic receptors are engaged in the control of insulin secretion, and their activation stimulates pancreas to secrete insulin. Hence, it can be expected that M3 receptors blockade may be unfavorable in terms of the risk of development of type II diabetes in patients treated with second generation antipsychotics (ex. olanzapine, clozapine, quetiapine). Recent research is focused on substances free of this undesired effect (Silvestre J. S., Prous J., Methods Find. Exp. Clin. Pharmacol.; 27(5), 289-304, 2005).

Another serious side effects caused by antipsychotic drugs, e.g. sertindole, ziprasidone, are cardiac arrhythmias associated with delayed repolarization of cardiomyocytes. This condition appears on electrocardiograms (ECG) as prolonged corrected QT interval (QTc), what is most often evoked by substances which block hERG potassium channels. To prevent introduction to the developmental pipelines drugs with proarrhythmic potential, at a very early stage of research new substances are screened in vitro for their potency to block hERG potassium channels, using electrophysiological methods (Recanatini M. et al., Med. Res. Rev., 25(2), 133-66, 2005).

Although introduction of new psychotropic drugs (among others neuroleptics, antidepressants, benzodiazepines, acetylocholinesterase inhibitors) since 50-thies of the XX century was an unquestioned breakthrough, therapy of neuropsychiatric disorders is still far from satisfactory both because of limited efficacy and wide spectrum of side effects evoked by available drugs. These disadvantages are a challenge for modern pharmacotherapy and there is a continuous effort to search for new, more effective psychotropic drugs.

Some sulphonamide derivatives of alicyclic amines are known in the art.

US2001/0034352 discloses sulphonamide derivatives of piperidine, useful for the treatment of diseases related to endothelial dysfunction.

In WO98/29411 some sulphonamide derivatives are disclosed, having affinity for 5-HT1A and D2, d3 and D4 receptors and useful for the treatment of CNS diseases.

Certain sulphonamide derivatives of alicyclic amines having hypotensive activity are known from U.S. Pat. No. 4,034,098.

EP976732A discloses compounds revealing serotonin antagonism and useful for treatment, ameliorating or preventing spastic paralysis or as central muscle relaxants for ameliorating myotonia.

In WO02/22579 sulphonamide heterocycles having antipsychotic activity are disclosed. These compounds are useful for treatment of diseases caused by abnormal activity of one or more GPCR-s or ligand-gated ion-channels, i.a. for the treatment of psychiatric disorders.

WO2007/110449, WO2007/118853 and WO 2009/040659 disclose benzenesulphonamide derivatives as calcium channel blockers, especially useful for the treatment of pain.

Further, in WO2006/105127 sulphonamide derivatives active as hydroxysteride dehydrogenase inhibitors.

EP1190710A relates to compounds, i.a. piperidine sulphonamides, useful for the treatment of diabetes.

WO03/087086 discloses a broad group of substituted indole derivatives for the prophylaxis and/or therapy of diseases in which 5HT plays a role, i.a. depression.

U.S. Pat. No. 5,739,135, U.S. Pat. Nos. 5,827,875 and 5,885,983 relate to compounds potentially useful as inhibitors of microsomal triglyceride transfer protein.

WO01/07436 discloses substituted oxoazaheterocyclyl compounds, which inhibit both factor Xa and Factor IIa, thus being useful in the treatment and prophylaxis of diseases relating to blood coagulation.

In WO2004/002490 piperidine derivatives for the treatment of bacterial infections in mammals were disclosed.

### AIM OF THE INVENTION

The aim of the present invention is to provide novel compounds potentially useful for the treatment of diseases of the central nervous system. A further aim of the invention is to provide novel compounds useful for the treatment of diseases of central nervous system having higher effectiveness compared to currently used medicaments. Yet further aim of the present invention is to provide novel compounds useful for the treatment of diseases of the central nervous system, which could allow to eliminate or minimize adverse effects associated with currently used therapies.

### DISCLOSURE OF THE INVENTION

The present invention relates to novel sulphonamide derivatives of alicyclic amines having the structure represented by the general formula (I)

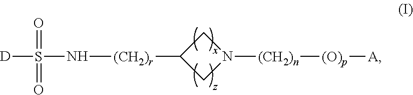

wherein
A represents naphthyl or 9- or 10-membered bicyclic group, linked to —(O)ₚ—(CH₂)ₙ— through one of its aromatic carbon atoms, consisting of benzene ring fused with:
5-membered heteroaromatic ring having 1 heteroatom selected from N and S or 2 heteroatoms independently selected from N, O, and S, wherein such a bicyclic group may be unsubstituted or substituted with halogen atom; or
5- or 6-membered heterocyclic non-aromatic ring having 1 or 2 heteroatoms independently selected from N and O, wherein heterocyclic ring may be unsubstituted or substituted with ═O or one or more C₁-C₃-alkyls;
   D represents a moiety selected from the group consisting of:
phenyl unsubstituted or substituted with one or more substituents independently selected from the group consisting of C₁-C₄-alkyl, C₁-C₃-alkyloxy, halogeno-C₁-C₃-alkyl, halogen atom, halogeno-C₁-C₃-alkyloxy-, —CN, —OH, and phenyl;
naphthyl unsubstituted or substituted with one or more substituents independently selected from the group consisting of C₁-C₄-alkyl, C₁-C₃-alkyloxy and halogen atom;
5-membered aromatic heterocyclic group having 1 or 2 heteroatoms independently selected from N, O, and S, unsubstituted or substituted with one or more substituents independently selected from the group consisting of C₁-C₄-alkyl, halogen atom, and 5-membered heteroaromatic ring having 1 or 2 heteroatoms independently selected from N and O, linked to sulphonamide group through one of its aromatic carbon atoms; and
bicyclic group consisting of a ring selected from benzene and pyridine, fused with 5-membered aromatic or non-aromatic heterocyclic ring, having 1 or 2 heteroatoms independently selected from N, O, and S, unsubstituted or substituted with one or more substitutents independently selected from the group consisting of C₁-C₄-alkyl, halogen atom, and ═O, linked to sulphonamide moiety through one of its aromatic carbon atoms;
   r represents 0 or 1;
   x and z represent independently 1 or 2;
   n represents 3 and p represents 0, or n represents 2 and p represents 1;
   and enantiomers, pharmaceutically acceptable salts and solvates thereof.

For one particular group of compounds of the present invention D represents a moiety selected from the group consisting of:
phenyl unsubstituted or substituted with one or more substituents independently selected from the group consisting of C₁-C₄-alkyl, C₁-C₃-alkyloxy, halogeno-C₁-C₃-alkyl, halogen atom, —CN, —OH, and phenyl;
naphthyl unsubstituted or substituted with one or more substituents independently selected from the group consisting of C₁-C₄-alkyl and halogen atom;
5-membered aromatic heterocyclic group having 1 or 2 heteroatoms independently selected from N, O, and S, unsubstituted or substituted with one or more substituents independently selected from the group consisting of C₁-C₄-alkyl, halogen atom, and 5-membered heteroaromatic ring having 1 or 2 heteroatoms independently selected from N and O; linked to sulphonamide group through one of its aromatic carbon atoms; and
bicyclic group consisting of a ring selected from benzene and pyridine, fused with 5-membered aromatic or non-aromatic heterocyclic ring, having 1 or 2 heteroatoms independently selected from N, O, S, unsubstituted or substituted with one or more substitutents independently selected from the group consisting of C₁-C₄-alkyl, halogen atom, and ═O, linked to sulphonamide moiety through one of its aromatic carbon atoms.

In one of embodiments of the present invention, A is linked to oxygen atom of —(O)ₚ—(CH₂)ₙ— moiety when p is 1, or to carbon atom of —(CH₂)ₙ— moiety when p is 0, through carbon atom of benzene ring. Preferably, when p is 1, then A is linked to oxygen atom of —(O)ₚ—(CH₂)ₙ— moiety through carbon atom of benzene ring.

In an alternative embodiment of the invention A is linked to oxygen atom of —(O)ₚ—(CH₂)ₙ— moiety when p is 1, or to carbon atom of —(O)ₚ—(CH₂)ₙ— moiety when p is 0, through carbon atom of heterocyclic ring. Preferably, when p is 0, then A is linked to carbon atom of —(O)ₚ—(CH₂)ₙ— moiety through carbon atom of heterocyclic ring.

Preferably, for compounds of formula (I) as described above, if A is linked to —(O)ₚ—(CH₂)ₙ— moiety through carbon atom of benzene ring, then n is 2 and p is 1, and if A is linked to —(O)ₚ—(CH₂)ₙ— moiety through carbon atom of 5-membered heteroaromatic ring, then n is 2 and p is 1, or n is 3 and p is 0.

One of variants of the compounds of the present invention are compounds of formula (I) wherein A represents naphthyl. Naphthyl may be linked to oxygen atom of —(O)ₚ—(CH₂)ₙ— moiety when p is 1, or to carbon atom of —(CH₂)ₙ— moiety when p is 0, through position 1 (alpha) or 2 (beta) of naphthyl ring. Preferred in the above variant are compounds (I) of the invention where A is naphthyl and is linked to oxygen atom of —(O)ₚ—(CH₂)ₙ— moiety (p=1).

Another group of compounds of the invention are compounds of formula (I), wherein A represents 9-membered bicyclic group consisting of benzene ring fused with 5-membered monoheteroaromatic ring having 1 heteroatom selected from N and S, preferably having N as heteroatom. In this case A may be linked to oxygen atom, of —(O)ₚ—(CH₂)ₙ— moiety when p is 1, or to carbon atom of —(O)ₚ—(CH₂)ₙ— moiety when p is 0, through carbon atom of benzene ring or through carbon atom of 5-membered heteroaromatic ring. Advantageously, in this case A is linked to oxygen atom of —(O)ₚ—(CH₂)ₙ— moiety when p is 1, through carbon atom of benzene ring, or to carbon atom of —(O)ₚ—(CH₂)ₙ— moiety when p is 0, through carbon atom of 5-membered heteroaromatic ring. Preferably A in this group represents 1H-indol-4-yl, 1H-indol-6-yl, or 1H-indol-3-yl, which may be optionally substituted with halogen atom. More preferably, A in this group represents 1H-indol-4-yl or 1H-indol-6-yl linked to oxygen atom of —(O)ₚ—(CH₂)ₙ— moiety (p=1), or 1H-indol-3-yl substituted with halogen atom and linked to carbon atom of —(CH₂)ₙ— moiety (p=0).

Further group of compounds of the present invention are the compounds of formula (I), wherein A represents 9-membered bicyclic group consisting of benzene ring fused with 5-membered heteroaromatic ring having 2 heteroatoms independently selected from N, O, and S. A may be linked to oxygen atom of —(O)ₚ—(CH₂)ₙ— moiety when p is 1, or to carbon atom of —(CH₂)ₙ— moiety when p is 0, through carbon atom of benzene ring or through carbon atom of 5-membered heteroaromatic ring, preferably through carbon atom of 5-membered heteroaromatic ring. Preferred A in this group of compounds is selected from 1,2-benzoxazol-3-yl and 1,2-benzothiazol-3-yl, which may be optionally substituted with halogen atom.

Another group of compounds of the present invention are the compounds of formula (I), wherein A represents 10-membered bicyclic group consisting of benzene ring fused with 6-membered heterocyclic ring having 1 or 2 heteroatoms independently selected from N and O. In this variant A may only be linked to oxygen atom of —(O)ₚ—(CH₂)ₙ— moiety when p is 1, or to carbon atom of —(CH₂)ₙ— moiety when p is 0, through carbon atom of benzene ring. Preferably in this variant A represents 1,4-benzodioxan-5-yl.

Yet another group of compounds of the present invention are the compounds of formula (I), wherein A represents 9-membered bicyclic group consisting of benzene ring fused with 5-membered heterocyclic non-aromatic having 1 or 2 heteroatoms independently selected from N and O, and wherein heterocyclic ring is substituted with ═O or with one or more C₁-C₃-alkyl. Preferably in this group of compounds A is selected from 1,3-dihydro-2H-indol-2-on-4-yl, 1,3-benzoxazol-2(3H)-on-7-yl, 1,3-benzoxazol-2(3H)-on-4-yl and 2,2-dimethyl-2,3-dihydro-1-benzofuran-7-yl.

Further group of compounds of the present invention are the compounds of formula (I), wherein D represents phenyl. Phenyl may be unsubstituted or substituted, as defined for substituent D above.

Yet another group of compounds of the invention are compounds of formula (I), wherein D represents naphthyl. Naphthyl may be linked to sulphur atom of sulphonamide moiety in position 1 (alpha) or 2 (beta) of naphthyl ring. Naphthyl may be unsubstituted or substituted, as defined for substituent D above, for example with halogen atom or C₁-C₃-alkyloxy. Preferably, naphthyl is unsubstituted.

Further group of compounds of the invention are compounds of formula (I), wherein D represents bicyclic group consisting of a ring selected from benzene and pyridine, fused with 5-membered aromatic or non-aromatic heterocyclic ring, having 1 or 2 heteroatoms independently selected from N, O, and S, unsubstituted or substituted with one or more substituents independently selected from the group consisting of C₁-C₄-alkyl, halogen atom, and ═O. Preferably, in this variant D is selected from the group consisting of 2,3-dihydrobenzofuran-6-yl, benzotiophen-2-yl, benzotiophen-3-yl, imidazo[1,2-a]pyridyn-3-yl, 1,3-benzothiazol-4-yl, and 1,3-benzothiazol-5-yl, which may be optionally substituted with halogen atom and/or C₁-C₃-alkyl.

Further variant of the compounds of formula (I) according to the invention are compounds wherein n is 3 and p is 0.

Another variant of the compounds of formula (I) according to the invention are compounds wherein n is 2 and p is 0.

Yet another group of the compounds of formula (I) according to the invention are compounds, wherein x and z are both 2. These group are therefore piperidine derivatives.

Further group of the compounds of formula (I) according to the invention are compounds wherein x is 2 and z is 1. These group are therefore pyrrolidine derivatives.

Yet further group of the compounds of formula (I) according to the invention are compounds wherein x and z are both 1. These group are therefore azetidine derivatives.

Another variant of the compounds of formula (I) of the present invention are compounds wherein r is 0.

Further variant of the compounds of formula (I) of the present invention are compounds wherein r is 1.

The following specific compounds of formula (I) of the invention can be mentioned:
1. N-[1-[3-(6-fluoro-1,2-benzoxazol-3-yl)propyl]azetidin-3-yl]benzene-sulphonamide,
2. 3-fluoro-N-[1-[3-(6-fluoro-1,2-benzoxazol-3-yl)propyl]azetidin-3-yl]benzene-sulphonamide,
3. 4-fluoro-N-[1-[3-(6-fluoro-1,2-benzoxazol-3-yl)propyl]azetidin-3-yl]benzene-sulphonamide,
4. 3-chloro-N-[1-[3-(6-fluoro-1,2-benzoxazol-3-yl)propyl]azetidin-3-yl]benzene-sulphonamide,
5. N-[1-[3-(6-fluoro-1,2-benzoxazol-3-yl)propyl]azetidin-3-yl]-3-methylbenzene-sulphonamide,
6. N-[1 [3-(6-fluoro-1,2-benzoxazol-3-yl)propyl]pyrrolidin-3-yl]benzenesulphonamide,
7. 3-fluoro-N-[1-[3-(6-fluoro-1,2-benzoxazol-3-yl)propyl]pyrrolidin-3-yl]benzene-sulphonamide,
8. 4-fluoro-N-[1-[3-(6-fluoro-1,2-benzoxazol-3-yl)propyl]pyrrolidin-3-yl]benzene-sulphonamide,
9. 3-chloro-N-[1-[3-(6-fluoro-1,2-benzoxazol-3-yl)propyl]pyrrolidin-3-yl]benzene-sulphonamide,
10. 4-bromo-N-[1-[3-(6-fluoro-1,2-benzoxazol-3-yl)propyl]pyrrolidin-3-yl]benzene-sulphonamide,
11. 4-chloro-3-fluoro-N-[1-[3-(6-fluoro-1,2-benzoxazol-3-yl)propyl]pyrrolidin-3-yl]benzenesulphonamide,
12. N-[1-[3-(6-fluoro-1,2-benzoxazol-3-yl)propyl]pyrrolidin-3-yl]-3-methylbenzene-sulphonamide,
13. N-[1-[3-(6-fluoro-1,2-benzoxazol-3-yl)propyl]pyrrolidin-3-yl]-4-propylbenzene-sulphonamide,
14. 4-tert-butyl-N-[1-[3-(6-fluoro-1,2-benzoxazol-3-yl)propyl]pyrrolidin-3-yl]-benzenesulphonamide,
15. N-[1-[3-(6-fluoro-1,2-benzoxazol-3-yl)propyl]pyrrolidin-3-yl]-3-(trifluoro-methyl)-benzenesulphonamide,
16. N-[1-[3-(6-fluoro-1,2-benzoxazol-3-yl)propyl]pyrrolidin-3-yl]-4-(trifluoro-methyl)-benzenesulphonamide,
17. N-[1-[3-(6-fluoro-1,2-benzoxazol-3-yl)propyl]pyrrolidin-3-yl]-3-methoxy-benzenesulphonamide,
18. N-[1-[3-(6-fluoro-1,2-benzoxazol-3-yl)propyl]pyrrolidin-3-yl]-3-hydroxy-benzenesulphonamide,
19. 3-cyano-N-[1-[3-(6-fluoro-1,2-benzoxazol-3-yl)propyl]pyrrolidin-3-yl]benzene-sulphonamide,
20. N-[1-[3-(6-fluoro-1,2-benzoxazol-3-yl)propyl]pyrrolidin-3-yl]naphthalene-1-sulphonamide,
21. N-[1-[3-(6-fluoro-1,2-benzoxazol-3-yl)propyl]pyrrolidin-3-yl]naphthalene-2-sulphonamide,
22. 5-chloro-N-[1-[3-(6-fluoro-1,2-benzoxazol-3-yl)propyl]pyrrolidin-3-yl]thiophene-2-sulphonamide,
23. 6-chloro-N-[1-[3-(6-fluoro-1,2-benzoxazol-3-yl)propyl]pyrrolidin-3-yl]naphthalene-2-sulphonamide,
24. N-[1-[3-(6-fluoro-1,2-benzoxazol-3-yl)propyl]pyrrolidin-3-yl]-2,3-dihydrobenzo-furano-6-sulphonamide,
25. N-[1-[3-(6-fluoro-1,2-benzoxazol-3-yl)propyl]pyrrolidin-3-yl]benzothiophene-2-sulphonamide,
26. N-[1 [3-(6-fluoro-1,2-benzoxazol-3-yl)propyl]pyrrolidin-3-yl]benzothiophene-3-sulphonamide,
27. 6-chloro-N-[1-[3-(6-fluoro-1,2-benzoxazol-3-yl)propyl]pyrrolidin-3-yl]benzo-thiophene-2-sulphonamide,
28. 5-fluoro-N-[1-[3-(6-fluoro-1,2-benzoxazol-3-yl)propyl]pyrrolidin-3-yl]-3-methyl-benzothiophene-2-sulphonamide,
29. 5-chloro-N-[1-[3-(6-fluoro-1,2-benzoxazol-3-yl)propyl]pyrrolidin-3-yl]-3-methyl-benzothiophene-2-sulphonamide,
30. N-[1-[3-(6-fluoro-1,2-benzoxazol-3-yl)propyl]pyrrolidin-3-yl]imidazo[1,2-a]-pyridine-3-sulphonamide,
31. N-[1-[3-(6-fluoro-1,2-benzoxazol-3-yl)propyl]pyrrolidin-3-yl]-1,3-benzothiazole-4-sulphonamide,
32. N-[1-[3-(6-fluoro-1,2-benzoxazol-3-yl)propyl]-4-piperidine]benzenesulphonamide,
33. N-[1-[3-(6-fluoro-1,2-benzoxazol-3-yl)propyl]-4-piperidine]-3-methylbenzene-sulphonamide,
34. N-[1-[3-(6-fluoro-1,2-benzoxazol-3-yl)propyl]-4-piperidine]naphthalene-1-sulphonamide,
35. N-[1-[3-(6-fluoro-1,2-benzoxazol-3-yl)propyl]-4-piperidine]naphthalene-2-sulphonamide,
36. N-[[1-[3-(6-fluoro-1,2-benzoxazol-3-yl)propyl]-4-piperidine]methyl]-3-methy-benzenesulphonamide,
37. N-[[1-[3-(6-fluoro-1,2-benzoxazol-3-yl)propyl]-4-piperidine]methyl]-naphthalene-1-sulphonamide,
38. N-[[1-[3-(6-fluoro-1,2-benzoxazol-3-yl)propyl]-4-piperidine]methyl]-naphthalene-2-sulphonamide,
39. N-[1-[2-(1,2-benzothiazol-3-yloxy)ethyl]pyrrolidin-3-yl]naphthalene-2-sulphonamide,
40. N-[1 [2-(1,2-benzothiazol-3-yloxy)ethyl]-4-piperidine]naphthalene-2-sulphonamide,
41. N-[[1-[2-(1,2-benzothiazol-3-yloxy)ethyl]azetidin-3-yl]methyl]-3-hydroxy-benzene-sulphonamide,
42. N-[[1-[2-(1,2-benzothiazol-3-yloxy)ethyl]azetidin-3-yl]methyl]naphthalene-2-sulphonamide,
43. N-[[1-[2-(1,2-benzothiazol-3-yloxy)ethyl]pyrrolidin-3-yl]methyl]-3-hydroxy-benzene-sulphonamide,
44. N-[[1-[2-(1,2-benzothiazol-3-yloxy)ethyl]pyrrolidin-3-yl]methyl]naphthalene-2-sulphonamide,
45. N-[1-[2-(1H-indol-4-yloxy)ethyl]azetidin-3-yl]benzenesulphonamide,
46. 4-fluoro-N-[1-[2-(1H-indol-4-yloxy)ethyl]azetidin-3-yl]benzenesulphonamide,
47. 3-chloro-N-[1-[2-(1 N-indol-4-yloxy)ethyl]azetidin-3-yl]benzenesulphonamide,
48. N-[1-[2-(1H-indol-4-yloxy)ethyl]azetidin-3-yl]-3-methyl-benzenesulphonamide,
49. N-[1-[2-(1H-indol-4-yloxy)ethyl]azetidin-3-yl]naphthalene-1-sulphonamide,
50. N-[1-[2-(l N-indol-4-yloxy)ethyl]azetidin-3-yl]naphthalene-2-sulphonamide,
51. N-[1-[2-(1H-indol-4-yloxy)ethyl]pyrrolidin-3-yl]benzenesulphonamide,
52. N-[1-[2-(1H-indol-4-yloxy)ethyl]pyrrolidin-3-yl]-3-methylbenzenesulphonamide,
53. N-[1-[2-(1H-indol-4-yloxy)ethyl]pyrrolidin-3-yl]naphthalene-1-sulphonamide,
54. N-[1-[2-(1H-indol-4-yloxy)ethyl]pyrrolidin-3-yl]naphthalene-2-sulphonamide,
55. N-[1-[2-(1H-indol-4-yloxy)ethyl]-4-piperidine]benzenesulphonamide,
56. N-[1-[2-(1H-indol-4-yloxy)ethyl]-4-piperidine]-3-methylbenzenesulphonamide,
57. 4-tert-butyl-N-[1-[2-(1H-indol-4-yloxy)ethyl]-4-piperidine]benzene-sulphonamide,
58. N-[1-[2-(1H-indol-4-yloxy)ethyl]-4-piperidine]-4-(trifluoromethyl)benzene-sulphonamide,
59. 4-cyano-N-[1-[2-(1H-indol-4-yloxy)ethyl]-4-piperidine]benzenesulphonamide,
60. N-[1-[2-(1H-indol-4-yloxy)ethyl]-4-piperidine]naphthalene-1-sulphonamide,
61. N-[1-[2-(1H-indo-4-yloxy)ethyl]-4-piperidine]naphthalene-2-sulphonamide,
62. 5-chloro-N-[1-[2-(1H-indo-4-yloxy)ethyl]-4-piperidine]-3-methylbenzo-thiophene-2-sulphonamide,
63. N-[[1-[2-(1H-indol-4-yloxy)ethyl]-4-piperidine]methyl]benzenesulphonamide,
64. N-[[1-[2-(1H-indol-4-yloxy)ethyl]-4-piperidine]methyl]-3-methyl-benzene-sulphonamide,
65. 3-hydroxy-N-[[1-[2-(1H-indol-4-yloxy)ethyl]-4-piperidine]methyl]benzene-sulphonamide,
66. N-[[1-[2-(1H-indol-4-yloxy)ethyl]-4-piperidine]methyl]naphthalene-1-sulphonamide,
67. N-[[1-[2-(1H-indol-4-yloxy)ethyl]-4-piperidine]methyl]naphthalene-2-sulphonamide,
68, N-[1-[2-(1H-indol-6-yloxy)ethyl]pyrrolidin-3-yl]benzenesulphonamide,
69. N-[1-[2-(1H-indol-6-yloxy)ethyl]pyrrolidin-3-yl]-3-methylbenzenesulphonamide,
70. N-[[1-[2-(1H-indol-6-yloxy)ethyl]-4-piperidine]methyl]benzenesulphonamide,
71. N-[[1-[2-(1H-indo-6-yloxy)ethyl]-4-piperidine]methyl]naphthalene-1-sulphonamide,
72. N-[[1-[2-(1H-indol-6-yloxy)ethyl]-4-piperidine]methyl]naphthalene-2-sulphonamide,
73. N-[1-[3-(5-fluoro-1H-indol-3-yl)propyl]pyrrolidin-3-yl]benzenesulphonamide,
74. 3-fluoro-N-[1-[3-(5-fluoro-1H-indo-3-yl)propyl]pyrrolidin-3-yl]benzene-sulphonamide,
75. 4-fluoro-N-[1-[3-(5-fluoro-1H-indol-3-yl)propyl]pyrrolidin-3-yl]benzene-sulphonamide,
76. 3-chloro-N-[1-[3-(5-fluoro-1H-indol-3-yl)propyl]pyrrolidin-3-yl]benzene-sulphonamide,
77. 4-chloro-N-[1-[3-(5-fluoro-1H-indol-3-yl)propyl]pyrrolidin-3-yl]benzene-sulphonamide,
78. N-[1-[3-(5-fluoro-1H-indol-3-yl)propyl]pyrrolidin-3-yl]-3-methyl-benzene-sulphonamide,
79. N-[1-[3-(5-fluoro-1H-indol-3-yl)propyl]pyrrolidin-3-yl]naphthalene-1-sulphonamide,
80. N-[1-[3-(5-fluoro-1H-indol-3-yl)propyl]pyrrolidin-3-yl]naphthalene-2-sulphonamide,
81. N-[1-[3-(5-chloro-1H-indol-3-yl)propyl]pyrrolidin-3-yl]benzenesulphonamide,
82. N-[1-[3-(5-chloro-1H-indol-3-yl)propyl]pyrrolidin-3-yl]-3-fluoro-benzene-sulphonamide,
83. N-[1-[3-(5-chloro-1-indol-3-yl)propyl]pyrrolidin-3-yl]-4-fluoro-benzene-sulphonamide,
84. 3-chloro-N-[1-[3-(5-chloro-1H-indol-3-yl)propyl]pyrrolidin-3-yl]benzene-sulphonamide,
85. 4-chloro-N-[1-[3-(5-chloro-1H-indol-3-yl)propyl]pyrrolidin-3-yl]benzene-sulphonamide,
86. N-[1-[3-(5-chloro-1H-indol-3-yl)propyl]pyrrolidin-3-yl]-3-methyl-benzene-sulphonamide,
87. N-[1-[3-(5-chloro-1H-indol-3-yl)propyl]pyrrolidin-3-yl]naphthalene-2-sulphonamide,
88. N-[1-[2-(2,3-dihydro-1,4-benzodioxan-8-yloxy)ethyl]azetidin-3-yl]benzene-sulphonamide,
89. N-[1-[2-(2,3-dihydro-1,4-benzodioxan-8-yloxy)ethyl]azetidin-3-yl]-3-fluoro-benzenesulphonamide,
90. N-[1-[2-(2,3-dihydro-1,4-benzodioxan-8-yloxy)ethyl]azetidin-3-yl]-4-fluoro-benzenesulphonamide,
91. 3-chloro-N-[1-[2-(2,3-dihydro-1,4-benzodioxan-8-yloxy)ethyl]azetidin-3-yl]-benzene-sulphonamide,
92. 4-chloro-N-[1-[2-(2,3-dihydro-1,4-benzodioxan-8-yloxy)ethyl]azetidin-3-yl]-benzenesulphonamide,
93. N-[1-[2-(2,3-dihydro-1,4-benzodioxan-8-yloxy)ethyl]azetidin-3-yl]-3-methyl-benzenesulphonamide,
94. N-[1-[2-(2,3-dihydro-1,4-benzodioxan-8-yloxy)ethyl]azetidin-3-yl]naphthalene-1-sulphonamide,
95. N-[1-[2-(2,3-dihydro-1,4-benzodioxan-8-yloxy)ethyl]azetidin-3-yl]naphthalene-2-sulphonamide,
96. N-[1-[2-(2,3-dihydro-1,4-benzodioxan-8-yloxy)ethyl]pyrrolidin-3-yl]benzene-sulphonamide,
97. N-[1-[2-(2,3-dihydro-1,4-benzodioxan-8-yloxy)ethyl]pyrrolidin-3-yl]-3-methyl-benzene-sulphonamide,
98. N-[1-[2-(2,3-dihydro-1,4-benzodioxan-8-yloxy)ethyl]pyrrolidin-3-yl]-naphthalene-1-sulphonamide
99. N-[1-[2-(2,3-dihydro-1,4-benzodioxan-8-yloxy)ethyl]pyrrolidin-3-yl]-naphthalene-2-sulphonamide,
100. N-[1-[2-(2,3-dihydro-1,4-benzodioxan-8-yloxy)ethyl]-4-piperidine]naphthalene-1-sulphonamide,
101. N-[[1-[2-(2,3-dihydro-1,4-benzodioxan-5-yloxy)ethyl]-4-piperidine]methyl]-benzenesulphonamide,
102. N-[[1-[2-(2,3-dihydro-1,4-benzodioxan-5-yloxy)ethyl]-4-piperidine]methyl]-3-methylbenzenesulphonamide,
103. N-[[1-[2-(2,3-dihydro-1,4-benzodioxan-5-yloxy)ethyl]-4-piperidine]methyl]-3-hydroxybenzenesulphonamide,
104. N-[[1-[2-(2,3-dihydro-1,4-benzodioxan-5-yloxy)ethyl]-4-piperidine]methyl]-naphthalene-1-sulphonamide,
105. N-[[1-[2-(2,3-dihydro-1,4-benzodioxan-5-yloxy)ethyl]-4-piperidine]methyl]-naphthalene-2-sulphonamide,
106. N-[[1-[2-(2-oxoindolin-4-yl)oxyethyl]pyrrolidin-3-yl]methyl]naphthalene-2-sulphonamide,
107. N-[1-[2-[(2,2-dimethyl-3H-benzofuran-7-yl)oxy]ethyl]pyrrolidin-3-yl]-3-hydroxy-benzenesulphonamide,
108. N-[1-[2-[(2,2-dimethyl-3H-benzofuran-7-yl)oxy]ethyl]pyrrolidin-3-yl]-naphthalene-2-sulphonamide,
109. N-[1-[2-[(2,2-dimethyl-3H-benzofuran-7-yl)oxy]ethyl]-4-piperidine]-3-hydroxy-benzene-sulphonamide,
110. N-[1-[2-[(2,2-dimethyl-3H-benzofuran-7-yl)oxy]ethyl]-4-piperidine]naphthalene-2-sulphonamide,
111. N-[[1-[2-[(2,2-dimethyl-3H-benzofuran-7-yl)oxy]ethyl]azetidin-3-yl]methyl]-3-hydroxy-benzenesulphonamide,
112. N-[[1-[2-[(2,2-dimethyl-3H-benzofuran-7-yl)oxy]ethyl]azetidin-3-yl]methyl]-naphthalene-2-sulphonamide,
113. N-[[1-[2-[(2,2-dimethyl-3H-benzofuran-7-yl)oxy]ethyl]pyrrolidin-3-yl]methyl]-3-hydroxy-benzenesulphonamide,
114. N-[[1-[2-[(2,2-dimethyl-3H-benzofuran-7-yl)oxy]ethyl]pyrrolidin-3-yl]methyl]-naphthalene-2-sulphonamide,
115. 3-hydroxy-N-[1-[2-(1-naphthyloxy)ethyl]pyrrolidin-3-yl]benzenesulphonamide,
116. N-[1-[2-(1-naphthyloxy)ethyl]pyrrolidin-3-yl]naphthalene-2-sulphonamide,
117. N-[1-[2-(2,3-dihydro-1,4-benzodioxan-8-yloxy)ethyl]-4-piperidine]benzene-sulphonamide,
118. N-[1-[2-(2,3-dihydro-1,4-benzodioxan-8-yloxy)ethyl]-4-piperidine]-3-methyl-benzenesulphonamide,
119. 3-hydroxy-N-[1-[2-(1-naphthyloxy)ethyl]-4-piperidine]benzenesulphonamide,
120. N-[1-[2-(1-naphthyloxy)ethyl]-4-piperidine]naphthalene-2-sulphonamide,
121. 3-hydroxy-N-[[1-[2-(1-naphthyloxy)ethyl]azetidin-3-yl]methyl]benzene-sulphonamide,
122. N-[[1-[2-(1-naphthyloxy)ethyl]azetidin-3-yl]methyl]naphthalene-2-sulphonamide,
123. 3-hydroxy-N-[[1-[2-(1-naphthyloxy)ethyl]pyrrolidin-3-yl]methyl]benzene-sulphonamide,
124. N-[[1-[2-(1-naphthyloxy)ethyl]pyrrolidin-3-yl]methyl]naphthalene-2-sulphonamide,
125. N-[1-[2-(1,2-benzothiazol-3-yloxy)ethyl]pyrrolidin-3-yl]-3-hydroxybenzene-sulphonamide,
126. N-[[1-[3-(6-fluoro-1,2-benzoxazol-3-yl)propyl]azetidin-3-yl]methyl]-1,3-benzodioxole-5-sulphonamide,
127. N-[[1-[3-(6-fluoro-1,2-benzoxazol-3-yl)propyl]azetidin-3-yl]methyl]benzo-thiophene-2-sulphonamide,
128. N-[[1-[3-(6-fluoro-1,2-benzoxazol-3-yl)propyl]azetidin-3-yl]methyl]-1,3-benzothiazole-4-sulphonamide,
129. 6-chloro-N-[[1-[3-(6-fluoro-1,2-benzoxazol-3-yl)propyl]azetidin-3-yl]-methyl]-naphthalene-2-sulphonamide,
130. 5-fluoro-N-[[1-[3-(6-fluoro-1,2-benzoxazol-3-yl)propyl]azetidin-3-yl]methyl]-3-methyl-benzothiophene-2-sulphonamide,
131. 5-chloro-N-[[1-[3-(6-fluoro-1,2-benzoxazol-3-yl)propyl]azetidin-3-yl]-methyl]-3-methyl-benzothiophene-2-sulphonamide,
132. N-[[1-[3-(6-fluoro-1,2-benzoxazol-3-yl)propyl]azetidin-3-yl]methyl]-1,3-benzothiazole-5-sulphonamide,
133. N-[[1-[3-(6-fluoro-1,2-benzoxazol-3-yl)propyl]azetidin-3-yl]methyl]-naphthalene-1-sulphonamide,
134. N-[[1-[3-(6-fluoro 1,2-benzoxazol-3-yl)propyl]azetidin-3-yl]methyl]-naphthalene-2-sulphonamide,
135. N-[[1. [3-(6-fluoro-1,2-benzoxazol-3-yl)propyl]azetidin-3-yl]methyl]-4-phenyl-benzenesulphonamide,
136. 4-chloro-N-[[1-[3-(6-fluoro-1,2-benzoxazol-3-yl)propyl]azetidin-3-yl]-methyl]benzenesulphonamide,
137. 3-chloro-N-[[1-[3-(6-fluoro-1,2-benzoxazol-3-yl)propyl]azetidin-3-yl]-methyl]benzenesulphonamide,
138. N-[[1-[3-(6-fluoro-1,2-benzoxazol-3-yl)propyl]azetidin-3-yl]methyl]-4-methyl-benzenesulphonamide,
139. N-[[1-[3-(6-fluoro-1,2-benzoxazol-3-yl)propyl]azetidin-3-yl]methyl]-benzene-sulphonamide,
140. N-[[1. [3-(6-fluoro-1,2-benzoxazol-3-yl)propyl]azetidin-3-yl]methyl]-4-(trifluoromethyl)benzenesulphonamide,
141. 4-tert-butyl-N-[[1-[3-(6-fluoro-1,2-benzoxazol-3-yl)propyl]azetidin-3-yl]methyl]benzenesulphonamide,
142. N-[[1-[3-(6-fluoro-1,2-benzoxazol-3-yl)propyl]azetidin-3-yl]methyl]-3-methyl-benzenesulphonamide,
143. N-[[1-[3-(6-fluoro 1,2-benzoxazol-3-yl)propyl]azetidin-3-yl]methyl]-3-methoxy-benzenesulphonamide,
144. 3-fluoro-N-[[1-[3-(6-fluoro-1,2-benzoxazol-3-yl)propyl]azetidin-3-yl]methyl]-benzenesulphonamide,
145. 4-cyano-N-[[1-[3-(6-fluoro-1,2-benzoxazol-3-yl)propyl]azetidin-3-yl]methyl]-benzenesulphonamide,
146. 3,4-dichloro-N-[[1-[3-(6-fluoro-1,2-benzoxazol-3-yl)propyl]azetidin-3-yl]-methyl]benzenesulphonamide,
147. 4-fluoro-N-[[1. [3-(6-fluoro 1,2-benzoxazol-3-yl)propyl]azetidin-3-yl]methyl]-benzenesulphonamide,
148. 4-bromo-N-[[1-[3-(6-fluoro-1,2-benzoxazol-3-yl)propyl]azetidin-3-yl]methyl]-benzenesulphonamide,
149. N-[[1-[3-(6-fluoro 1,2-benzoxazol-3-yl)propyl]azetidin-3-yl]methyl]-3-hydroxy-benzenesulphonamide, N-[[1-[3-(6-fluoro 1,2-benzoxazol-3-yl)propyl]azetidin-3-yl]methyl]-1-methyl-indole-5-sulphonamide,
151. N-[[1-[3-(6-fluoro-1,2-benzoxazol-3-yl)propyl]azetidin-3-yl]methyl]-benzofuran-2-sulphonamide,
152. N-[[1-[3-(6-fluoro-1,2-benzoxazol-3-yl)propyl]azetidin-3-yl]methyl]-1-methyl-indole-4-sulphonamide,
153. N-[[1-[3-(6-fluoro-1,2-benzoxazol-3-yl)propyl]azetidin-3-yl]methyl]-benzo-thiophene-2-sulphonamide,
154. N-[[1-[3-(6-fluoro-1,2-benzoxazol-3-yl)propyl]azetidin-3-yl]methyl]-thiophene-3-sulphonamide,
155. 5-chloro-N-[[1-[3-(6-fluoro-1,2-benzoxazol-3-yl)propyl]azetidin-3-yl]methyl]-thiophene-2-sulphonamide,
156. 3-chloro-4-fluoro-N-[[1-[3-(6-fluoro-1,2-benzoxazol-3-yl)propyl]azetidin-3-yl]-methyl]benzenesulphonamide,
157. N-[[1-[3-(6-fluoro-1,2-benzoxazol-3-yl)propyl]azetidin-3-yl]methyl]-4-propyl-benzenesulphonamide,
158. 3,4-difluoro-N-[[1-[3-(6-fluoro-1,2-benzoxazol-3-yl)propyl]azetidin-3-yl]-methyl]benzenesulphonamide,
159. N-[[1-[3-(6-fluoro-1,2-benzoxazol-3-yl)propyl]azetidin-3-yl]methyl]-4-(trifluoro-methoxy)benzenesulphonamide,
160. N-[[1-[3-(5-fluoro-1H-indol-3-yl)propyl]azetidin-3-yl]methyl]naphthalene-2-sulphonamide,
161. N-[[1-[3-(5-chloro-1 I-indol-3-yl)propyl]azetidin-3-yl]methyl]naphthalene-2-sulphonamide,
162. N-[[1-[2-(1,2-benzothiazol-3-yloxy)ethyl]azetidin-3-yl]methyl]-naphthalene-2-sulphonamide,
163. N-[[1-[2-(1-naphthyloxy)ethyl]azetidin-3-yl]methyl]benzothiophene-2-sulphonamide,
164. 6-chloro-N-[[1-[2-(1-naphthyloxy)ethyl]azetidin-3-yl]methyl]-benzothiophene-2-sulphonamide,
165. 6-chloro-N-[[1-[2-(1-naphthyloxy)ethyl]azetidin-3-yl]methyl]naphthalene-2-sulphonamide,
166. 5-fluoro-3-methyl-N-[[1-[2-(1-naphthyloxy)ethyl]azetidin-3-yl]methyl]benzo-thiophene-2-sulphonamide,
167. 5-chloro-3-methyl-N-[[1-[2-(1-naphthyloxy)ethyl]azetidin-3-yl]methyl]benzo-thiophene-2-sulphonamide,
168. N-[[1-[2-(1-naphthyloxy)ethyl]azetidin-3-yl]methyl]naphthalene-1-sulphonamide,
169. 1-methyl-N-[[1-[2-(1-naphthyloxy)ethyl]azetidin-3-yl]methyl]indole-5-sulphonamide,
170. 1-methyl-N-[[1-[2-(1-naphthyloxy)ethyl]azetidin-3-yl]methyl]indol-4-sulphonamide,
171. N-[[1-[2-(1-naphthyloxy)ethyl]azetidin-3-yl]methyl]benzothiophene-3-sulphonamide,
172. 3-chloro-4-fluoro-N-[[1-[2-(1-naphthyloxy)ethyl]azetidin-3-yl]methyl]benzene-sulphonamide,
173. 3,4-difluoro-N-[[1-[2-(1-naphthyloxy)ethyl]azetidin-3-yl]methyl]-benzene-sulphonamide,
174. 6-chloro-N-[[1-[2-(2,3-dihydro-1,4-benzodioxin-5-yloxy)ethyl]azetidin-3-yl]-methyl]naphthalene-2-sulphonamide,
175. 5-chloro-N-[[1-[2-(2,3-dihydro-1,4-benzodioxin-5-yloxy)ethyl]azetidin-3-yl]-methyl]-3-methyl-benzothiophene-2-sulphonamide,
176. N-[[1-[2-(2,3-dihydro-1,4-benzodioxin-5-yloxy)ethyl]azetidin-3-yl]methyl]-naphthalene-1-sulphonamide,
177. N-[[1-[2-(2,3-dihydro-1,4-benzodioxin-5-yloxy)ethyl]azetidin-3-yl]methyl]-naphthalene-2-sulphonamide,
178. N-[[1-[2-(2,3-dihydro-1,4-benzodioxin-5-yloxy)ethyl]azetidin-3-yl]methyl]-4-phenyl-benzenesulphonamide,
179. 4-chloro-N-[[1-[2-(2,3-dihydro-1,4-benzodioxin-5-yloxy)ethyl]azetidin-3-yl]methyl]benzenesulphonamide,
180. N-[[1-[2-(2,3-dihydro-1,4-benzodioxin-5-yloxy)ethyl]azetidin-3-yl]methyl]-4-(trifluoromethyl)benzenesulphonamide,
181. 4-tert-butyl-N-[[1-[2-(2,3-dihydro-1,4-benzodioxin-5-yloxy)ethyl]azetidin-3-yl]methyl]benzenesulphonamide,
182. N-[[1-[2-(2,3-dihydro-1,4-benzodioxin-5-yloxy)ethyl]azetidin-3-yl]methyl]-4-fluoro-benzenesulphonamide
183. 3,4-dichloro-N-[[1-[2-(2,3-dihydro-1,4-benzodioxin-5-yloxy)ethyl]azetidin-3-yl]methyl]benzenesulphonamide,
184. N-[[1-[2-(2,3-dihydro-1,4-benzodioxin-5-yloxy)ethyl]azetidin-3-yl]methyl]-thiophene-2-sulphonamide,
185. 4-bromo-N-[[1-[2-(2,3-dihydro-1,4-benzodioxin-5-yloxy)ethyl]azetidin-3-yl]methyl]benzenesulphonamide,
186. N-[[1-[2-(2,3-dihydro-1,4-benzodioxin-5-yloxy)ethyl]azetidin-3-yl]methyl]-benzofuran-2-sulphonamide,
187. N-[[1-[2-(2,3-dihydro-1,4-benzodioxin-5-yloxy)ethyl]azetidin-3-yl]methyl]-1-methyl-indole-5-sulphonamide,
188. N-[[1-[2-(2,3-dihydro-1,4-benzodioxin-5-yloxy)ethyl]azetidin-3-yl]methyl]-1-methyl-indole-4-sulphonamide,
189. N-[[1-[2-(2,3-dihydro-1,4-benzodioxin-5-yloxy)ethyl]azetidin-3-yl]methyl]-2-oxo-indoline-5-sulphonamide,
190. N-[[1-[2-(2,3-dihydro-1,4-benzodioxin-5-yloxy)ethyl]azetidin-3-yl]methyl]-benzothiophene-3-sulphonamide,
191. N-[[1-[2-(2,3-dihydro-1,4-benzodioxin-5-yloxy)ethyl]azetidin-3-yl]methyl]-thiophene-3-sulphonamide,
192. 5-chloro-N-[[1-[2-(2,3-dihydro-1,4-benzodioxin-5-yloxy)ethyl]azetidin-3-yl]methyl]thiophene-2-sulphonamide,
193. N-[[1-[2-(2,3-dihydro-1,4-benzodioxin-5-yloxy)ethyl]azetidin-3-yl]methyl]-4-iodo-benzenesulphonamide,
194. N-[1-[3-(6-fluoro-1,2-benzoxazol-3-yl)propyl]pyrrolidin-3-yl]-1,3-benzo-dioxole-5-sulphonamide,
195. N-[1-[3-(6-fluoro-1,2-benzoxazol-3-yl)propyl]pyrrolidin-3-yl]-4-phenyl-benzenesulphonamide,
196. N-[(3R)-1-[3-(6-fluoro-1,2-benzoxazol-3-yl)propyl]pyrrolidin-3-yl]benzo-thiophene-2-sulphonamide,
197. N-[(3S)-1-[3-(6-fluoro-1,2-benzoxazol-3-yl)propyl]pyrrolidin-3-yl]benzo-thiophene-2-sulphonamide,
198. 6-chloro-N-[(3R)-1-[3-(6-fluoro-1,2-benzoxazol-3-yl)propyl]pyrrolidin-3-yl]-benzothiophene-2-sulphonamide,
199. 6-chloro-N-[(3S)-1-[3-(6-fluoro-1,2-benzoxazol-3-yl)propyl]pyrrolidin-3-yl]-benzothiophene-2-sulphonamide,
200. 6-chloro-N-[(3R)-1-[3-(6-fluoro-1,2-benzoxazol-3-yl)propyl]pyrrolidin-3-yl]-naphthalene-2-sulphonamide,
201. 6-chloro-N-[(3S)-1-[3-(6-fluoro-1,2-benzoxazol-3-yl)propyl]pyrrolidin-3-yl]-naphthalene-2-sulphonamide,
202. 5-fluoro-N-[(3R)-1-[3-(6-fluoro-1,2-benzoxazol-3-yl)propyl]pyrrolidin-3-yl]-3-methyl-benzothiophene-2-sulphonamide,
203. 5-fluoro-N-[(3S)-1-[3-(6-fluoro-1,2-benzoxazol-3-yl)propyl]pyrrolidin-3-yl]-3-methyl-benzothiophene-2-sulphonamide,
204. 5-chloro-N-[(3R)-1-[3-(6-fluoro-1,2-benzoxazol-3-yl)propyl]pyrrolidin-3-yl]-3-methyl-benzothiophene-2-sulphonamide,
205. 5-chloro-N-[(3S)-1-[3-(6-fluoro-1,2-benzoxazol-3-yl)propyl]pyrrolidin-3-yl]-3-methyl-benzothiophene-2-sulphonamide,
206. N-[(3R)-1-[3-(6-fluoro-1,2-benzoxazol-3-yl)propyl]pyrrolidin-3-yl]naphthalene-2-sulphonamide,
207. N-[(3S)-1-[3-(6-fluoro-1,2-benzoxazol-3-yl)propyl]pyrrolidin-3-yl]-naphthalene-2-sulphonamide,
208. 4-chloro-N-[1-[3-(6-fluoro-1,2-benzoxazol-3-yl)propyl]pyrrolidin-3-yl]-benzenesulphonamide,
209. N-[1-[3-(6-fluoro-1,2-benzoxazol-3-yl)propyl]pyrrolidin-3-yl]-4-methyl-benzene-sulphonamide,
210. 4-cyano-N-[1-[3-(6-fluoro-1,2-benzoxazol-3-yl)propyl]pyrrolidin-3-yl]-benzene-sulphonamide
211. 3,4-dichloro-N-[1-[3-(6-fluoro-1,2-benzoxazol-3-yl)propyl]pyrrolidin-3-yl]-benzenesulphonamide,
212. N-[1-[3-(6-fluoro-1,2-benzoxazol-3-yl)propyl]pyrrolidin-3-yl]thiophene-2-sulphonamide,
213. N-[1-[3-(6-fluoro-1,2-benzoxazol-3-yl)propyl]pyrrolidin-3-yl]-4-methoxy-benzenesulphonamide,
214. N-[(3R)-1-[3-(6-fluoro-1,2-benzoxazol-3-yl)propyl]pyrrolidin-3-yl]benzo-furan-2-sulphonamide,
215. N-[(3S)-1-[3-(6-fluoro-1,2-benzoxazol-3-yl)propyl]pyrrolidin-3-yl]benzo-furan-2-sulphonamide,
216. N-[1-[3-(6-fluoro-1,2-benzoxazol-3-yl)propyl]pyrrolidin-3-yl]benzofuran-2-sulphonamide,
217. N-[1-[3-(6-fluoro-1,2-benzoxazol-3-yl)propyl]pyrrolidin-3-yl]-1-methyl-imidazole-4-sulphonamide,
218. N-[1-[3-(6-fluoro-1,2-benzoxazol-3-yl)propyl]pyrrolidin-3-yl]-1-methyl-indole-5-sulphonamide,
219. N-[1-[3-(6-fluoro-1,2-benzoxazol-3-yl)propyl]pyrrolidin-3-yl]-1-methyl-indole-4-sulphonamide,
220. N-[1-[3-(6-fluoro-1,2-benzoxazol-3-yl)propyl]pyrrolidin-3-yl]-2-oxo-indoline-5-sulphonamide,
221. N-[1-[3-(6-fluoro-1,2-benzoxazol-3-yl)propyl]pyrrolidin-3-yl]-2,5-dimethyl-thiophene-3-sulphonamide,
222. N-[(3R)-1-[3-(6-fluoro-1,2-benzoxazol-3-yl)propyl]pyrrolidin-3-yl]-2,5-dimethyl-thiophene-3-sulphonamide,
223. N-[(3S)-1-[3-(6-fluoro-1,2-benzoxazol-3-yl)propyl]pyrrolidin-3-yl]-2,5-dimethyl-thiophene-3-sulphonamide,
224. N-[(3R)-1-[3-(6-fluoro-1,2-benzoxazol-3-yl)propyl]pyrrolidin-3-yl]-benzo-thiophene-3-sulphonamide
225. N-[(3S)-1-[3-(6-fluoro-1,2-benzoxazol-3-yl)propyl]pyrrolidin-3-yl]-benzo-thiophene-3-sulphonamide,
226. N-[1-[3-(6-fluoro-1,2-benzoxazol-3-yl)propyl]pyrrolidin-3-yl]-5-methyl-benzo-thiophene-2-sulphonamide,
227. N-[1-[3-(6-fluoro-1,2-benzoxazol-3-yl)propyl]pyrrolidin-3-yl]-6-methoxy-naphthalene-2-sulphonamide,
228. N-[(3R)-1-[3-(6-fluoro-1,2-benzoxazol-3-yl)propyl]pyrrolidin-3-yl]-5-methyl-benzothiophene-2-sulphonamide,
229. N-[(3S)-1-[3-(6-fluoro-1,2-benzoxazol-3-yl)propyl]pyrrolidin-3-yl]-5-methyl-benzothiophene-2-sulphonamide,
230. N-[(3R)-1-[3-(6-fluoro-1,2-benzoxazol-3-yl)propyl]pyrrolidin-3-yl]-6-methoxy-naphthalene-2-sulphonamide,
231. N-[(3S)-1-[3-(6-fluoro-1,2-benzoxazol-3-yl)propyl]pyrrolidin-3-yl]-6-methoxy-naphthalene-2-sulphonamide,
232. 7-chloro-N-[(3R)-1-[3-(6-fluoro-1,2-benzoxazol-3-yl)propyl]pyrrolidin-3-yl]naphthalene-2-sulphonamide,
233. 7-chloro-N-[(3S)-1-[3-(6-fluoro-1,2-benzoxazol-3-yl)propyl]pyrrolidin-3-yl]naphthalene-2-sulphonamide,
234. 6-fluoro-N-[1-[3-(6-<fluoro-1,2-benzoxazol-3-yl)propyl]pyrrolidin-3-yl]benzo-thiophene-2-sulphonamide,
235. N-[[1-[3-(6-fluoro-1,2-benzoxazol-3-yl)propyl]pyrrolidin-3-yl]methyl]-1,3-benzodioxole-5-sulphonamide,
236. N-[[1-[3-(6-fluoro-1,2-benzoxazol-3-yl)propyl]pyrrolidin-3-yl]methyl]-1,3-benzothiazole-4-sulphonamide,
237. 6-chloro-N-[[1-[3-(6-fluoro-1,2-benzoxazol-3-yl)propyl]pyrrolidin-3-yl]methyl]-naphthalene-2-sulphonamide,
238. 5-chloro-N-[[1-[3-(6-fluoro-1,2-benzoxazol-3-yl)propyl]pyrrolidin-3-yl]methyl]-3-methyl-benzothiophene-2-sulphonamide,
239. N-[[1-[3-(6-fluoro-1,2-benzoxazol-3-yl]propyl]pyrrolidin-3-yl)methyl]-naphthalene-1-sulphonamide,
240. N-[[1-[3-(6-fluoro-1,2-benzoxazol-3-yl)propyl]pyrrolidin-3-yl]methyl]-naphthalene-2-sulphonamide,
241. N-[[1-[3-(6-fluoro-1,2-benzoxazol-3-yl)propyl]pyrrolidin-3-yl]methyl]-4-phenyl-benzenesulphonamide,
242. 4-chloro-N-[[1-[3-(6-fluoro-1,2-benzoxazol-3-yl)propyl]pyrrolidin-3-yl]methyl]-benzenesulphonamide,
243. 3-chloro-N-[[1-[3-(6-fluoro-1,2-benzoxazol-3-yl)propyl]pyrrolidin-3-yl]methyl]-benzenesulphonamide,
244. N-[[1-[3-(6-fluoro-1,2-benzoxazol-3-yl)propyl]pyrrolidin-3-yl]methyl]-4-methyl-benzenesulphonamide,
245. N-[[1-[3-(6-fluoro-1,2-benzoxazol-3-yl)propyl]pyrrolidin-3-yl]methyl]-benzenesulphonamide,
246. N-[[1-[3-(6-fluoro-1,2-benzoxazol-3-yl)propyl]pyrrolidin-3-yl]methyl]-4-(trifluoromethyl)benzenesulphonamide,
247. N-[[1-[3-(6-fluoro 1,2-benzoxazol-3-yl)propyl]pyrrolidin-3-yl]methyl]-3-(trifluoromethyl)benzenesulphonamide,
248. 4-tert-butyl-N-[[1-[3-(6-fluoro-1,2-benzoxazol-3-yl)propyl]pyrrolidin-3-yl]-methyl]benzenesulphonamide,
249. 3-tert-butyl-N-[[1-[3-(6-fluoro-1,2-benzoxazol-3-yl)propyl]pyrrolidin-3-yl]-methyl]benzenesulphonamide,
250. N-[[1-[3-(6-fluoro-1,2-benzoxazol-3-yl)propyl]pyrrolidin-3-yl]methyl]-3-methoxy-benzenesulphonamide,
251. 4-cyano-N-[[1-[3-(6-fluoro-1,2-benzoxazol-3-yl)propyl]pyrrolidin-3-yl]-methyl]benzenesulphonamide,
252. 4-fluoro-N-[[1-[3-(6-fluoro-1,2-benzoxazol-3-yl)propyl]pyrrolidin-3-yl]methyl]-benzenesulphonamide,
253. 3,4-dichloro-N-[[1-[3-(6-fluoro-1,2-benzoxazol-3-yl)propyl]pyrrolidin-3-yl]-methyl]benzenesulphonamide,
254. N-[[1-[3-(6-fluoro-1,2-benzoxazol-3-yl)propyl]pyrrolidin-3-yl]methyl]-3-hydroxy-benzenesulphonamide,
255. N-[[1-[3-(6-fluoro-1,2-benzoxazol-3-yl)propyl]pyrrolidin-3-yl]methyl]-4-methoxy-benzenesulphonamide,
256. N-[[1-[3-(6-fluoro-1,2-benzoxazol-3-yl)propyl]pyrrolidin-3-yl]methyl]-2,3-dihydrobenzofuran-5-sulphonamide,
257. N-[[1-[3-(6-fluoro-1,2-benzoxazol-3-yl)propyl]pyrrolidin-3-yl]methyl]-benzofuran-2-sulphonamide,
258. N-[[1-[3-(6-fluoro-1,2-benzoxazol-3-yl)propyl]pyrrolidin-3-yl]methyl]-1-methyl-indole-5-sulphonamide,
259. N-[[1-[3-(6-fluoro-1,2-benzoxazol-3-yl)propyl]pyrrolidin-3-yl]methyl]-1-methyl-indole-4-sulphonamide,
260. N-[[1-[3-(6-fluoro-1,2-benzoxazol-3-yl)propyl]pyrrolidin-3-yl]methyl]-2-oxo-indoine-5-sulphonamide,
261. N-[[1-[3-(6-fluoro-1,2-benzoxazol-3-yl)propyl]pyrrolidin-3-yl]methyl]benzo-thiophene-3-sulphonamide,
262. N-[[1-[3-(6-fluoro-1,2-benzoxazol-3-yl)propyl]pyrrolidin-3-yl]methyl]-2,5-dimethylthiophene-3-sulphonamide,
263. 3-chloro-4-fluoro-N-[[1-[3-(6-fluoro-1,2-benzoxazol-3-yl)propyl]pyrrolidin-3-yl]methyl]benzenesulphonamide,
264. N-[[1-[3-(6-fluoro-1,2-benzoxazol-3-yl)propyl]pyrrolidin-3-yl]methyl]-4-propyl-benzenesulphonamide,
265. 3,4-difluoro-N-[[1-[3-(6-fluoro-1,2-benzoxazol-3-yl)propyl]pyrrolidin-3-yl]-methyl]benzenesulphonamide,
266. N-[[1-[3-(6-fluoro-1,2-benzoxazol-3-yl)propyl]pyrrolidin-3-yl]methyl]-4-(trifluoromethoxy)benzenesulphonamide,
267. N-[[1-[3-(5-fluoro-1H-indol-3-yl)propyl]pyrrolidin-3-yl]methyl]-naphthalene-2-sulphonamide,
268. N-[[1-[2-(1-naphthyloxy)ethyl]pyrrolidin-3-yl]methyl]benzothiophene-2-sulphonamide,
269. 6-chloro-N-[[1-[2-(1-naphthyloxy)ethylpyrrolidin-3-yl]methyl]-benzothiophene-2-sulphonamide,
270. 6-chloro-N-[[1-[2-(1-naphthyloxy)ethyl]pyrrolidin-3-yl]methyl]-naphthalene-2-sulphonamide,
271. 5-fluoro-3-methyl-N-[[1-[2-(1-naphthyloxy)ethyl]pyrrolidin-3-yl]methyl]-benzothiophene-2-sulphonamide,
272. 5-chloro-3-methyl-N-[[1-[2-(1-naphthyloxy)ethyl]pyrrolidin-3-yl]methyl]-benzothiophene-2-sulphonamide,
273. N-[[1-[2-(1-naphthyloxy)ethyl]pyrrolidin-3-yl]methyl]naphthalene-1-sulphonamide,
274. 1-methyl-N-[[1-[2-(1-naphthyloxy)ethyl]pyrrolidin-3-yl]methyl]indole-5-sulphonamide,
275. 1-methyl-N-[[1-[2-(1-naphthyloxy)ethyl]pyrrolidin-3-yl]methyl]indole-4-sulphonamide,
276. N-[[1-[2-(1-naphthyloxy)ethyl]pyrrolidin-3-yl]methyl]benzothiophene-3-sulphonamide,
277. 3-chloro-4-fluoro-N-[[1-[2-(1-naphthyloxy)ethyl]pyrrolidin-3-yl]methyl]-benzenesulphonamide,
278. 3,4-difluoro-N-[[1-[2-(1-naphthyloxy)ethyl]pyrrolidin-3-yl]methyl]-benzene-sulphonamide,
279. N-[[1-[2-(2,3-dihydro-1,4-benzodioxin-5-yloxy)ethyl]pyrrolidin-3-yl]methyl]-1,3-benzodioxole-5-sulphonamide,
280. N-[[1-[2-(2,3-dihydro-1,4-benzodioxin-5-yloxy)ethyl]pyrrolidin-3-yl]methyl]-benzothiophene-2-sulphonamide,
281. N-[[1-[2-(2,3-dihydro-1,4-benzodioxin-5-yloxy)ethyl]pyrrolidin-3-yl]methyl]-1,3-benzothiazole-4-sulphonamide,
282. 6-chloro-N-[[1. [2-(2,3-dihydro-1,4-benzodioxin-5-yloxy)ethyl]pyrrolidin-3-yl]-methyl]naphthalene-2-sulphonamide,
283. 5-chloro-N-[[1-[2-(2,3-dihydro-1,4-benzodioxin-5-yloxy)ethyl]pyrrolidin-3-yl]-methyl]-3-methyl-benzothiophene-2-sulphonamide,
284. N-[[1-[2-(2,3-dihydro-1,4-benzodioxin-5-yloxy)ethyl]pyrrolidin-3-yl]methyl]-thiazole-2-sulphonamide,
285. N-[[1-[2-(2,3-dihydro-1,4-benzodioxin-5-yloxy)ethyl]pyrrolidin-3-yl]methyl]-1,3-benzothiazole-5-sulphonamide,
286. N-[[1-[2-(2,3-dihydro-1,4-benzodioxin-5-yloxy)ethyl]pyrrolidin-3-yl]methyl]-naphthalene-1-sulphonamide,
287. N-[[1-[2-(2,3-dihydro-1,4-benzodioxin-5-yloxy)ethyl]pyrrolidin-3-yl]methyl]-naphthalene-2-sulphonamide,
288. N-[[1-[2-(2,3-dihydro-1,4-benzodioxin-5-yloxy)ethyl]pyrrolidin-3-yl]methyl]-4-phenyl-benzenesulphonamide,
289. 4-chloro-N-[[1-[2-(2,3-dihydro-1,4-benzodioxin-5-yloxy)ethyl]pyrrolidin-3-yl]-methyl]benzenesulphonamide,
290. 3-chloro-N-[[1-[2-(2,3-dihydro-1,4-benzodioxin-5-yloxy)ethyl]pyrrolidin-3-yl]-methyl]benzenesulphonamide,
291. N-[[1-[2-(2,3-dihydro-1,4-benzodioxin-5-yloxy)ethyl]pyrrolidin-3-yl]methyl]-4-methyl-benzenesulphonamide,
292. N-[[1-[2-(2,3-dihydro-1,4-benzodioxin-5-yloxy)ethyl]pyrrolidin-3-yl]methyl]-benzenesulphonamide,
293. N-[[1-[2-(2,3-dihydro-1,4-benzodioxin-5-yloxy)ethyl]pyrrolidin-3-yl]methyl]-4-(trifluoromethyl)benzenesulphonamide,
294. N-[[1-[2-(2,3-dihydro-1,4-benzodioxin-5-yloxy)ethyl]pyrrolidin-3-yl]methyl]-3-(trifluoromethyl)benzenesulphonamide,
295. 4-tert-butyl-N-[[1-[2-(2,3-dihydro-1,4-benzodioxin-5-yloxy)ethyl]pyrrolidin-3-yl]methyl]benzenesulphonamide,
296. N-[[1-[2-(2,3-dihydro-1,4-benzodioxin-5-yloxy)ethyl]pyrrolidin-3-yl]methyl]-3-methylbenzenesulphonamide,
297. N-[[1-[2-(2,3-dihydro-1,4-benzodioxin-5-yloxy)ethyl]pyrrolidin-3-yl]methyl]-3-methoxybenzenesulphonamide,
298. N-[[1-[2-(2,3-dihydro-1,4-benzodioxin-5-yloxy)ethyl]pyrrolidin-3-yl]methyl]-3-fluoro-benzenesulphonamide,
299. 4-cyano-N-[[1-[2-(2,3-dihydro-1,4-benzodioxin-5-yloxy)ethyl]pyrrolidin-3-yl]-methyl]benzenesulphonamide,
300. N-[[1-[2-(2,3-dihydro-1,4-benzodioxin-5-yloxy)ethyl]pyrrolidin-3-yl]methyl]-4-fluoro-benzenesulphonamide,
301. 3,4-dichloro-N-[[1-[2-(2,3-dihydro-1,4-benzodioxin-5-yloxy)ethyl]pyrrolidin-3-yl]methyl]benzenesulphonamide,
302. N-[[1-[2-(2,3-dihydro-1,4-benzodioxin-5-yloxy)ethyl]pyrrolidin-3-yl]methyl]-thiophene-2-sulphonamide,
303. N-[[1-[2-(2,3-dihydro-1,4-benzodioxin-5-yloxy)ethyl]pyrrolidin-3-yl]methyl]-3-hydroxybenzenesulphonamide,
304. N-[[1-[2-(2,3-dihydro-1,4-benzodioxin-5-yloxy)ethyl]pyrrolidin-3-yl]methyl]-4-methoxybenzenesulphonamide,
305. 4-bromo-N-[[1-[2-(2,3-dihydro-1,4-benzodioxin-5-yloxy)ethyl]pyrrolidin-3-yl]methyl]benzenesulphonamide,
306. N-[[1-[2-(2,3-dihydro 1,4-benzodioxin-5-yloxy)ethyl]pyrrolidin-3-yl]methyl]-2,3-dihydrobenzofuran-5-sulphonamide,
307. N-[[1-[2-(2,3-dihydro-1,4-benzodioxin-5-yloxy)ethyl]pyrrolidin-3-yl]methyl]-benzofuran-2-sulphonamide,
308. N-[[1-[2-(2,3-dihydro-1,4-benzodioxin-5-yloxy)ethyl]pyrrolidin-3-yl]methyl]-1-methyl-indole-5-sulphonamide,
309. N-[[1-[2-(2,3-dihydro-1,4-benzodioxin-5-yloxy)ethyl]pyrrolidin-3-yl]methyl]-1-methyl-indole-4-sulphonamide,
310. N-[[1-[2-(2,3-dihydro-1,4-benzodioxin-5-yloxy)ethyl]pyrrolidin-3-yl]methyl]-2-oxo-indoline-5-sulphonamide,
311. N-[[1-[2-(2,3-dihydro-1,4-benzodioxin-5-yloxy)ethyl]pyrrolidin-3-yl]methyl]-benzothiophene-3-sulphonamide,
312. N-[[1-[2-(2,3-dihydro-1,4-benzodioxin-5-yloxy)ethyl]pyrrolidin-3-yl]methyl]-2,5-dimethyl-thiophene-3-sulphonamide,
313. N-[[1-[2-(2,3-dihydro-1,4-benzodioxin-5-yloxy)ethyl]pyrrolidin-3-yl]methyl]-thiophene-3-sulphonamide,
314. N-[[1-[2-(2,3-dihydro-1,4-benzodioxin-5-yloxy)ethyl]pyrrolidin-3-yl]methyl]-5-isoxazol-5-yl-thiophene-2-sulphonamide,
315. 3-cyano-N-[[1-[2-(2,3-dihydro-1,4-benzodioxin-5-yloxy)ethyl]pyrrolidin-3-yl]-methyl]benzenesulphonamide,
316. 5-chloro-N-[[1-[2-(2,3-dihydro-1,4-benzodioxin-5-yloxy)ethyl]pyrrolidin-3-yl]-methyl]thiophene-2-sulphonamide,
317. 3-chloro-N-[[1-[2-(2,3-dihydro-1,4-benzodioxin-5-yloxy)ethyl]pyrrolidin-3-yl]-methyl]-4-fluorobenzenesulphonamide,
318. N-[[1-[2-(2,3-dihydro-1,4-benzodioxin-5-yloxy)ethyl]pyrrolidin-3-yl]methyl]-4-propylbenzenesulphonamide,
319. N-[[1-[2-(2,3-dihydro-1,4-benzodioxin-5-yloxy)ethyl]pyrrolidin-3-yl]methyl]-3,4-difluoro-benzenesulphonamide,
320. N-[[1-[2-(2,3-dihydro-1,4-benzodioxin-5-yloxy)ethyl]pyrrolidin-3-yl]methyl]-4-(trifluoromethoxy)benzenesulphonamide,
321. N-[[1-[2-(2,3-dihydro-1,4-benzodioxin-5-yloxy)ethyl]pyrrolidin-3-yl]methyl]-4-iodobenzenesulphoniamide,
322. 3-bromo-N-[[1-[2-(2,3-dihydro-1,4-benzodioxin-5-yloxy)ethyl]pyrrolidin-3-yl]methyl]benzenesulphonamide,
323. N-[[1-[2-(2,3-dihydro-1,4-benzodioxin-5-yloxy)ethyl]pyrrolidin-3-yl]methyl]-5-methyl-isoxazole-4-sulphonamide,
324. N-[1-[3-(6-fluoro-1,2-benzoxazol-3-yl)propyl]-4-piperidyl]benzothiophene-2-sulphonamide
325. 6-chloro-N-[1-[3-(6-fluoro-1,2-benzoxazol-3-yl)propyl]-4-piperidyl]-naphthalene-2-sulphonamide,
326. 5-chloro-N-[1-[3-(6-fluoro-1,2-benzoxazol-3-yl)propyl]-4-piperidyl]-3-methyl-benzothiophene-2-sulphonamide,
327. N-[1-[3-(6-fluoro-1,2-benzoxazol-3-yl)propyl]-4-piperidyl]-4-phenylbenzene-sulphonamide,
328. 4-tert-butyl-N-[1-[3-(6-fluoro-1,2-benzoxazol-3-yl)propyl]-4-piperidyl]-benzenesulphonamide,
329. N-[1-[3-(6-fluoro-1,2-benzoxazol-3-yl)propyl]-4-piperidyl]-1-methyl-indole-4-sulphonamide,
330. N-[1-[3-(6-fluoro-1,2-benzoxazol-3-yl)propyl]-4-piperidyl]benzothiophene-3-sulphonamide,
331. N-[1-[3-(5-chloro-1H-indol-3-yl)propyl]pyrrolidin-3-yl]benzothiophene-2-sulphonamide,
332. 6-chloro-N-[1-[3-(5-chloro-1H-indol-3-yl)propyl]pyrrolidin-3-yl]benzothiophene-2-sulphonamide,
333. 6-chloro-N-[1-[3-(5-chloro-1 N-indol-3-yl)propyl]pyrrolidin-3-yl]naphthalene-2-sulphonamide,
334. N-[1-[3-(5-chloro-1H-indol-3-yl)propyl]pyrrolidin-3-yl]-5-fluoro-3-methyl-benzothiophene-2-sulphonamide,
335. 5-chloro-N-[1-[3-(5-chloro-1 N-indol-3-yl)propyl]pyrrolidin-3-yl]-3-methy-benzothiophene-2-sulphonamide,
336. 3,4-dichloro-N-[1-[3-(5-chloro-1H-indol-3-yl)propyl]pyrrolidin-3-yl]benzenesulphonamide,
337. N-[1-[3-(5-chloro-1H-indol-3-yl)propyl]pyrrolidin-3-yl]thiophene-2-sulphonamide,
338. N-[1-[3-(5-chloro-1H-indol-3-yl)propyl]pyrrolidin-3-yl]-1-methyl-indole-5-sulphonamide,
339. N-[1-[3-(5-chloro-1H-indol-3-yl)propyl]pyrrolidin-3-yl]-1-methyl-indole-4-sulphonamide,
340. N-[1-[3-(5-chloro-1H-indol-3-yl)propyl]pyrrolidin-3-yl]benzothiophene-3-sulphonamide,
341. N-[1-[3-(5-chloro-1H-indol-3-yl)propyl]pyrrolidin-3-yl]naphthalene-1-sulphonamide,
342. 3-chloro-N-[1-[3-(5-chloro-1H-indol-3-yl)propyl]pyrrolidin-3-yl]-4-fluoro-benzenesulphonamide,
343. N-[1-[3-(5-chloro-1H-indol-3-yl)propyl]pyrrolidin-3-yl]-3,4-difluoro-benzenesulphonamide,
344. N-[1-[3-(5-fluoro-1H-indol-3-yl)propyl]pyrrolidin-3-yl]benzothiophene-2-sulphonamide,
345. 6-chloro-N-[1-[3-(5-fluoro-1H-indol-3-yl)propyl]pyrrolidin-3-yl]benzothiophene-2-sulphonamide,
346. 6-chloro-N-[1-[3-(5-fluoro-1H-indol-3-yl)propyl]pyrrolidin-3-yl]naphthalene-2-sulphonamide,
347. 5-fluoro-N-[1-[3-(5-fluoro-1H-indol-3-yl)propyl]pyrrolidin-3-yl]-3-methy-benzothiophene-2-sulphonamide,
348. 5-chloro-N-[1-[3-(5-fluoro-1H-indol-3-yl)propyl]pyrrolidin-3-yl]-3-methyl-benzothiophene-2-sulphonamide,
349. 3,4-dichloro-N-[1-[3-(5-fluoro-1H-indol-3-yl)propyl]pyrrolidin-3-yl]benzenesulphonamide,
350. N-[1-[3-(5-fluoro-1-indol-3-yl)propyl]pyrrolidin-3-yl]thiophene-2-sulphonamide,
351. N-[1-[3-(5-fluoro-1H-indol-3-yl)propyl]pyrrolidin-3-yl]-1-methyl-indole-5-sulphonamide,
352. N-[1-[3-(5-fluoro-1 N-indol-3-yl)propyl]pyrrolidin-3-yl]-1-methyl-indole-4-sulphonamide,
353. N-[1-[3-(5-fluoro-1H-indol-3-yl)propyl]pyrrolidin-3-yl]benzothiophene-3-sulphonamide,
354. 3-chloro-4-fluoro-N-[1-[3-(5-fluoro-1H-indol-3-yl)propyl]pyrrolidin-3-yl]benzenesulphonamide,
355. 3,4-difluoro-N-[1-[3-(5-fluoro-1H-indol-3-yl)propyl]pyrrolidin-3-yl]benzenesulphonamide,
   and enantiomers, pharmaceutically acceptable salts and solvates thereof.

Sulphonamide derivatives of alicyclic amines of the above formula (I) exhibit affinity for receptors which are recognized therapeutical targets in the treatment of CNS disorders, such as dopaminergic, in particular D2 and D3, serotoninergic, in particular 5-HT1A, 5-HT2A, 5-HT6, 5-HT7, adrenergic, in particular α1 and α2C, and to serotonin transporter receptors. They have low affinity for biological targets associated with adverse effects, such as muscarinic receptors M3, histaminergic receptors H1 or serotoninergic receptors 5-HT2C. Due to such a broad pharmacological profile, the compounds of the invention may be useful in medicine as medicaments, for the treatment and/or prevention of the central nervous system disorders such as schizophrenia, schizoaffective disorders, schizophreniform disorders, delusional syndromes and other psychotic conditions related and not related to taking psychoactive substances, depression, affective bipolar disorder, mania and depression episodes, anxiety disorders of various etiology, conciousness disorders including coma, delirium of alcoholic or other etiology, aggression, psychomotor agitation and other conduct disorders, sleep disorders of various etiology, withdrawal syndromes of various etiology, addiction, pain syndromes of various etiology, intoxication with psychoactive substances, cerebral circulatory disorders of various etiology, psychosomatic disorders of various etiology, conversion disorders, dissociative disorders, urination disorders, autism and other developmental disorders, including nocturia, stuttering, tics, cognitive disorders of various types, such as Alzheimer's disease, psychopatological symptoms and neurological disorders in the course of other diseases of the central and peripheral nervous systems.

Thus, the subject of the present invention are the compounds of formula (I) as defined above, for use as a medicament.

In the treatment of central nervous system disorders compounds of formula (I) may be administered in the form of a pharmaceutical composition or preparation containing it.

Thus, the subject of the present invention is also the pharmaceutical composition containing the compound or compounds of formula (I) as defined above as an active substance, in combination with pharmaceutically acceptable carrier(s) and/or excipient(s).

The subject of the invention are also sulphonamide derivatives of the above formula (I) for use in the treatment of disorders of central nervous system.

The invention relates also to a method for the treatment of disorders of the central nervous system in mammals, including humans, comprising administration of a therapeutically effective amount of the compound of above formula (I) or the pharmaceutical composition containing the compound of formula (I) as defined above as an active substance.

Terms used in the description of the present invention have the following meanings.

Unless otherwise indicated, the term “C₁-C₄-alkyl” relates to a saturated, straight or branched hydrocarbon group, having indicated number of carbon atoms. Specific examples of groups encompassed by this term are methyl, ethyl, n-propyl, isopropyl, n-butyl, tert-butyl and sec-butyl.

The term “C₁-C₃-alkyloxy” relates to —O—C₁-C₃-alkyl group, wherein C₁-C₃-alkyl relates to a saturated, straight or branched hydrocarbon group, having indicated number of carbon atoms. Specific examples of groups encompassed by this term are methoxy, ethoxy, n-propoxy, isopropoxy.

The term “halogen atom” relates to a substituent selected from F, Cl, Br and I.

The term “halogeno-C₁-C₃-alkyl” relates to a saturated, straight or branched hydrocarbon group, having indicated number of carbon atoms and in which one carbon atom may be substituted with from 1-3 halogen atoms, depending on the number of carbon atoms bonded to it. Halogen atom has the meaning as defined above. Particularly preferred example of a group encompassed by this term is trifluoromethyl group —CF₃.

The term “halogeno-C₁-C₃-alkyloxy” relates to —O—C₁-C₃-halogenoalkyl group, wherein C₁-C₃-halogenoalkyl relates to a saturated, straight or branched hydrocarbon group, having indicated number of carbon atoms and in which one carbon atom may be substituted with from 1-3 halogen atoms, depending on the number of carbon atoms bonded to it. Halogen atom has the meaning as defined above. Particularly preferred example of a group encompassed by this term is trifluoromethoxy group —O—CF₃.

The compounds of formula (I) according to the invention can be prepared in a process presented in the following scheme:

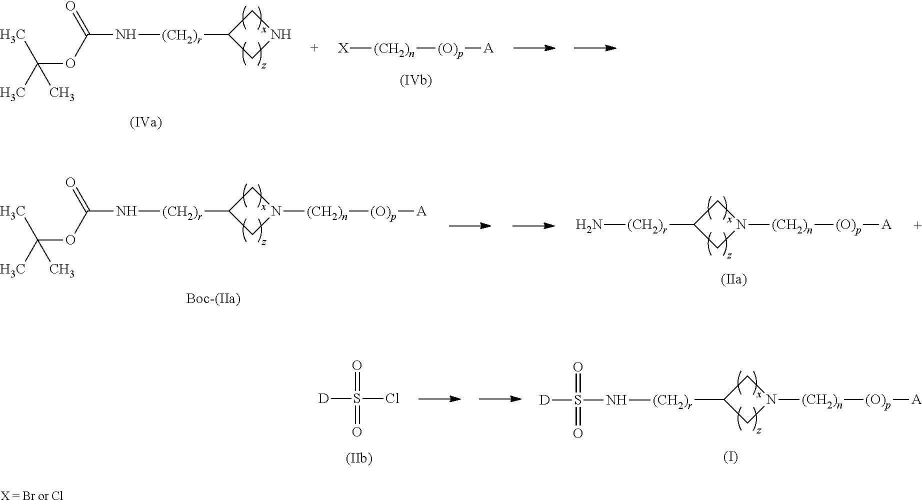

In the first step, an appropriate diamine having Boc-protected (tert-butyl carboxylate) primary amino group (IVa) is subjected to nucleophillic substitution reaction with an appropriate halogen derivative (IVb) in a solvent, for example in acetonitrite, in the presence of a base, for example triethylamine and/or potassium carbonate, at elevated temperature, for example at the boiling point of the solvent, to afford a derivative of formula (III). Product of the substitution reaction, amine Boc-(IIA), is deprotected using 4M solution of hydrogen chloride in dioxane or using a solution of trifluoroacetic acid in methylene chloride. The resulting amine (IIa) is reacted with sulfonyl chloride (IIb) in a solvent, for example N,N-dimethylformamide or methylene chloride, in the presence of a base, for example diisopropylethylamine, pyridine, or cesium carbonate, and 4-dimethylaminopyridine (DMAP) to give sulphonamide derivative of alicyclic amine (I) according to the invention.

Starting materials of formulas (IVa), (IVb) and (IIb) are either well known or commercially available, or can be prepared from commercially available starting materials by adapting and applying known methods.

Preparation of exemplary starting compounds of formula (IIa) is described in detail in the experimental part.

Since the compounds of formula (I) have alkaline character (contain at least one tertiary amine group), they can form acid addition salts.

Salts with acids can be pharmaceutically acceptable, especially when they are intended to be an active ingredient in a pharmaceutical composition. The present invention relates also to salts of the compounds of formula (I) with acids other than pharmaceutically acceptable ones, which may be useful for example as intermediates suitable for purification of the compounds of the invention. In practice, it is often desirable to isolate first the compound from a reaction mixture in the form of a salt which is not pharmaceutically acceptable to purify the compound, and then convert the salt into free base by treatment with alkaline agent and to isolate, and optionally convert into the salt again.

Acid addition salts can be formed with inorganic (mineral) or organic acids. In particular, hydrochloric, hydrobromic, hydroiodic, phosphoric, sulphuric, nitric, carbonic, succinic, maleic, formic, acetic, propionic, fumaric, citric, tartaric, lactic, benzoic, salicylic, glutamic, aspargic, p-toluenesulphonic, benzenesulphonic, methanesulphonic, ethanesulphonic, naphthalenesulphonic such as 2-naphthalene-sulphonic, pamoic, xinafoic or hexanoic acids can be mentioned as examples of acids.

Acid addition salt can be prepared in a simple manner by reaction of the compound of formula (I) with suitable inorganic or organic acid, optionally in suitable solvent, such as organic solvent, to form a salt that is usually isolated, for example by crystallization and filtration. For example, compounds in the form of a free base can be converted into corresponding hydrochloride salts by reaction of a compound in a solution, for example in methanol, with stoichiometric amount of hydrochloric acid or with solution of hydrochloric acid in methanol, ethanol or diethyl ether, followed by evaporation of solvent(s).

The term “disorders of the central nervous system” should be understood as including disorders selected from schizophrenia, schizoaffective disorders, schizophreniform disorders, delusional syndromes and other psychotic conditions related and not related to taking psychoactive substances, affective disorder, bipolar disorder, mania, depression, anxiety disorders of various etiology, stress reactions, conciousness disorders, coma, delirium of alcoholic and other etiology, aggression, psychomotor agitation and other conduct disorders, sleep disorders of various etiology, withdrawal syndromes of various etiology, addiction, pain syndromes of various etiology, intoxication with psychoactive substances, cerebral circulatory disorders of various etiology, psychosomatic disorders of various etiology, conversion disorders, dissociative disorders, urination disorders, autism and other developmental disorders, including nocturia, stuttering, and tics, cognitive disorders of various types, like Alzheimer's disease, psychopathological symptoms and neurological disorders in the course of other diseases of the central and peripheral nervous systems.

In the treatment of the disorders mentioned above, compounds of formula (I) of the present invention can be administered as a chemical compound, but usually will be applied in the form of a pharmaceutical compositions containing the compound of the present invention or its pharmaceutically acceptable salt as defined above as an active ingredient in combination with pharmaceutically acceptable carrier(s) and/or excipient(s).

In the treatment of the above mentioned disorders the pharmaceutical compositions of the invention can be delivered by any route of administration, preferably oral or parenteral, and will have the form of a preparation for use in medicine, depending on the intended route of administration.

Compositions for oral administration may have the form of solid or liquid preparations. Solid preparations may be in the form, for example, tablets or capsules prepared in conventional manner using pharmaceutically acceptable inactive ingredients, such as binding agents (e.g. pregelatinized maize starch, polyvinylpyrrolidone or hydroxypropylmethylcellulose); fillers (e.g. lactose, sucrose, carboxymethylcellulose, microcrystalline cellulose or calcium hydrogen phosphate) lubricants (e.g. magnesium stearate, talc or silica); disintegrants (e.g. crospovidone, maize starch or sodium starch glycolate); wetting agents (e.g. sodium lauryl sulfate). The tablets may be coated using methods well known in the art with conventional coatings, delaying/controlling release coatings or enteric coatings. Liquid preparations for oral administration may have the form of e.g. solutions, syrups or suspensions, or may be prepared from a dry product suitable for reconstitution with water or other suitable carrier ex tempore. Such liquid preparations may be prepared by conventional methods with pharmaceutically acceptable inactive ingredients, such as suspending agents (e.g. sorbitol syrup, cellulose derivatives or hydrogenated edible fats), emulsifying agents (e.g. lecithin or acacia gum), non-aqueous matrix components (e.g. almond oil, oils esters, ethyl alcohol or fractionated vegetable oils) and preservatives (e.g. methyl or propyl p-hydroxybenzoates or sorbic acid). The preparations may also contain suitable buffering systems, flavouring and aroma agents, colourants and sweeteners.

Preparations for oral administration can be formulated according to methods well known to those skilled in the art to afford a controlled release of the active compound.

The parenteral route of administration comprises administration by intramuscular and intravenous injections and intravenous continuous infusions. Compositions for parenteral administration may be in the form of a dosage unit, e.g. in ampoules or in multidose containers with the addition of a preservative. The compositions may be in the form of suspensions, solutions or emulsions in oily or aqueous media, and may contain pharmaceutically acceptable excipients, such as suspending agents, stabilizing and/or dispersing agents. Alternatively, the active ingredient may be in the form of a powder for reconstitution ex tempore in a suitable carrier, e.g. sterile pyrogen-free water.

Method of treatment using compounds of this invention will be based on administration of a therapeutically effective amount of the compound of the invention, preferably in the form of a pharmaceutical composition, to a subject in need of such a treatment.

The proposed dose of the compounds of the invention will be comprised in the range from 1 to about 1000 mg per day, in a single dose or in divided doses. It will be apparent to those skilled in the art that selection of a dose required to achieve the desired biological effect will depend on several factors, such as the type of specific compound, the indication, route of administration, age and condition of a patient and the exact dose will be finally determined at the discretion of attending physician.

### EXAMPLE 1

### Preparation of Starting Compounds of Formula IIa

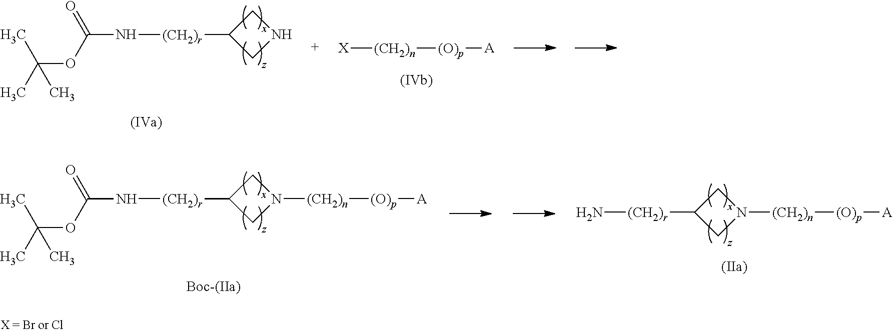

### 1a) Procedure for Halogen Derivative (IVb) Wherein X is Br and p=1

The amine (IVa) (1 mmol), bromoderivative (IVb) (1 mmol) and potassium carbonate (1.5 mmol) were stirred in acetonitrile (50 ml) under reflux overnight. Then the inorganic precipitate was filtered off, the filtrate was concentrated under reduced pressure and the residue was purified by column chromatography on silica gel using methylene chloride methanol 100:0 to 95:5 v/v as eluent.

Then the resulting protected amine Boc-(IIa) was subjected to deprotection according to one of the following procedures.

### 1a-1) Procedure for Deprotection of Amines Boc-(IIa) where r=0

To amine Boc-(IIIa) (0.5 mmol) 20 ml of methylene chloride and 5 ml of trifluoroacetic acid were added and the mixture was stirred at room temperature for 1 hour. Then the solvent was evaporated under reduced pressure and the product amine (IIa) as trifluoroacetic acid salt was used in the next step without purification.

### 1a-2) Procedure for Deprotection of Amines Boc-(IIa) where r=1

To amine Boc-(IIa) (0.5 mmol) 20 ml of methylene chloride and 5 ml of trifluoroacetic acid were added and the mixture was stirred at room temperature for 1 hour. Then the solvent was evaporated under reduced pressure and to the residue saturated aqueous sodium bicarbonate solution was added and then the mixture was extracted with ethyl acetate. After drying the organic phase over anhydrous magnesium sulfate, the residue after evaporation was purified by column chromatography on silica gel using methylene chloride/methanol 100:0-90:10 v/v as eluent to afford amine (IIa).

### 1b) Procedure for Halogen Derivatives (IVb) where X Represents Cl

A mixture of halogen derivative (IVb) (2.43 mmol), amine (IVa) (2.68 mmol), potassium carbonate (5.36 mmol), triethylamine (5.36 mmol) in acetonitrile (15 mL) was stirred at 70° C. for 16 hours. Then the inorganic precipitate was filtered off, the filtrate was concentrated under reduced pressure and the residue was purified by column chromatography on silica gel using methylene chloride/methanol 95:5 v/v as eluent. Then the resulting protected amine Boc-(IIa) was deprotected according to the following procedure.

Amine Boc-(IIa) (1.73 mmol) and 4M solution of hydrogen chloride in dioxane (10 ml) were stirred at room temperature for 45 min. Then dioxane was removed under reduced pressure and the residue was dried under vacuum for 1 hour to afford amine (IIa) as hydrochloride. The product was used directly in the next step without further purification.

Yields of amines (IIa) were in the range of 70-90%, and HPLC purities in the range of 90-95%.

Structure of prepared compounds was confirmed by MS analysis.

### Starting Amines (IVa):

tert-butyl azetidin-3-ylcarbamate (IVa-1),
tert-butyl pyrrolidin-3-ylcarbamate (IVa-2),
tert-butyl piperidin-4-ylcarbamate (IVa-3),
tert-butyl (azetidin-3-ylmethyl)carbamate (IVa-4),
tert-butyl (pyrrolidin-3-ylmethyl)carbamate (IVa-5),
tert-butyl (piperidin-4-ylmethyl)carbamate (IVa-6),
tert-butyl (3R)-pyrrolidin-3-ylcarbamate (IVa-7),
tert-butyl (3S)-pyrrolidin-3-ylcarbamate (IVa-8).

### Starting Halogen Derivatives (IVb):

3-(3-chloropropyl)-6-fluoro-1,2-benzoxazol (IVb-1),
3-(2-bromoethoxy)-1,2-benzothiazol (IVb-2),
4-(2-bromoethoxy)-1H-indole (IVb-3),
6-(2-bromoethoxy)-1H-indole (IVb-4),
3-(3-chloropropyl)-5-fluoro-1H-indole (IVb-5),
3-(3-chloropropyl)-5-chloro-1H-indole (IVb-6),
5-(2-bromoethoxy)-2,3-dihydro-1,4-benzodioxane (IVb-7),
4-(2-bromoethoxy)-1,3-dihydro-2H-indol-2-one (IVb-8),
7-(2-bromoethoxy)-2,2-dimethyl-2,3-dihydro-1-benzofuran (IVb-9),
1-(2-bromoethoxy)naphthalene (IVb-10).

Starting from appropriate amines (IVa) and halogen derivatives (IVb), the following amines (IIa) were prepared:
1-[3-(6-fluoro-1,2-benzoxazol-3-yl)propyl]azetidine-3-amine (IIa-1), hydrochloride; MS: 250 [M+H⁺],
1-[3-(6-fluoro-1,2-benzoxazol-3-yl)propyl]pyrrolidine-3-amine (IIa-2), hydrochloride; MS: 264 [M+H⁺],
1-[3-(6-fluoro-1,2-benzoxazol-3-yl)propyl]piperidine-4-amine (IIa-3), hydrochloride; MS: 278 [M+H⁺],
1-{1-[3-(6-fluoro-1,2-benzoxazol-3-yl)propyl]piperidin-4-yl}methaneamine (IIa-4), hydrochloride; MS: 292 [M+H⁺],
N-[2-(1,2-benzothiazol-3-yloxy)ethyl]pyrrolidine-3-amine (IIa-5), trifluoroacetate; MS: 264 [M+H⁺],
1-[2-(1,2-benzothiazol-3-yloxy)ethyl]piperidine-4-amine (IIa-6), trifluoroacetate; MS: 278 [M+H⁺],
1-{1-[2-(1,2-benzothiazol-3-yloxy)ethyl]azetidin-3-yl}methaneamine (IIIa-7), MS: 264 [M+H⁺],
1-{1-[2-(1,2-benzothiazol-3-yloxy)ethyl]pyrrolidin-3-yl}methaneamine (IIa-8), MS: 278 [M+H⁺],
1-[2-(1H-indol-4-yloxy)ethyl]azetidine-3-amine (IIa-9), trifluoroacetate; MS: 232 [M+H⁺],
1-[2-(1H-indol-4-yloxy)ethyl]pyrrolidine-3-amine (IIa-10), trifluoroacetate; MS: 246 [M+H⁺],
1-[2-(1H-indol-4-yloxy)ethyl]piperidine-4-amine (IIa-11), trifluoroacetate; MS: 260 [M+H⁺],
1-{1-[2-(1H-indol-4-yloxy)ethyl]piperidin-4-yl}methaneamine (IIa-12), MS: 274 [M+H⁺],
1-[2-(1H-indol-6-yloxy)ethyl]pyrrolidine-3-amine (IIa-13), MS: 246 [M+H⁺],
1-{1-[2-1H-indol-6-yloxy)ethyl]piperidin-4-yl}methaneamine (IIa-14), trifluoroacetate; MS: 274 [M+H⁺],
1-[3-(5-fluoro-1H-indol-3-yl)propyl]pyrrolidine-3-amine (IIa-15), hydrochloride; MS: 262 [M+H⁺],
1-[3-(5-chloro-1H-indol-3-yl)propyl]pyrrolidine-3-amine (IIa-16), hydrochloride; MS: 278 [M+H⁺],
1-[2-(2,3-dihydro-1,4-benzodioxan-5-yloxy)ethyl]azetidine-3-amine (IIa-17), trifluoroacetate; MS: 251 [M+H⁺],
1-[2-(2,3-dihydro-1,4-benzodioxan-5-yloxy)ethyl]pyrrolidine-3-amine (IIa-18), trifluoroacetate; MS: 265 [M+H⁺],
1-[2-(2,3-dihydro-1,4-benzodioxan-5-yloxy)ethyl]piperidine-4-amine (IIa-19), trifluoroacetate; MS: 279 [M+H⁺],
4-{2-[3-(aminomethyl)pyrrolidin-1-ylo]etoksy}-1,3-dihydro-2H-indol-2-on (IIa-21), MS: 276 [M+H⁺],
1-{2-[(2,2-dimethyl-2,3-dihydro-1-benzofuran-7-yl)oxy]ethyl}pyrrolidine-3-amine (IIa-22), trifluoroacetate; MS: 277 [M+H⁺],
1-{2-[(2,2-dimethyl-2,3-dihydro-1-benzofuran-7-yl)oxy]ethyl}piperidine-4-amine (IIa-23), trifluoroacetate; MS: 291 [M+H⁺],
1-(1-{2-[(2,2-dimethyl-2,3-dihydro-1-benzofuran-7-yl)oxy]ethyl}azetidin-3-yl)methaneamine (IIa-24), MS: 277 [M+H⁺],
1-(1-{2-[2,2-dimethyl-2,3-dihydro-1-benzofuran-7-yloxy]ethyl}pyrrolidin-3-yl)methaneamine (IIa-25), MS: 291 [M±H],
1-[2-(naphthalen-1-yloxy)ethyl]pyrrolidine-3-amine (IIa-26), trifluoroacetate; MS: 257 [M+H⁺],
1-[2-(naphthalen-1-yloxy)ethyl]piperidine-4-amine (IIa-27), trifluoroacetate; MS: 271 [M+H⁺],
1-{1-[2-(2,3-dihydro-1,4-benzodioxan-5-yloxy)ethyl]piperidin-4-yl}methaneamine (IIa-20), MS: 293 [M+H⁺],
1-{1-[2-(naphthalen-1-yloxy)ethyl]azetidin-3-yl}methaneamine (IIa-28), MS: 257 [M+H⁺],
1-{1-[2-(naphthalen-1-yloxy)ethyl]pyrrolidin-3-yl}methaneamine (IIa-29), MS: 271 [M+H⁺],
1-{1-[3-(6-fluoro-1,2-benzoxazol-3-yl)propyl]azetidin-3-yl}methaneamine (IIa-30), hydrochloride; MS: 264 [M+H⁺],
1-{1-[3-(6-fluoro-1,2-benzoxazol-3-yl)propyl]pyrrolidin-3-yl}methaneamine (IIa-31), hydrochloride; MS: 278 [M+H⁺],
(3R)-1-[3-(6-fluoro-1,2-benzoxazol-3-yl)propyl]pyrrolidine-3-amine (IIa-32), hydrochloride; MS: 264 [M+H⁺],
(3S)-1-[3-(6-fluoro-1,2-benzoxazol-3-yl)propyl]pyrrolidine-3-amine (IIa-33), hydrochloride; MS: 264 [M+H⁺],
1-{1-[3-(5-fluoro-1H-indol-3-yl)propyl]azetidin-3-yl}methaneamine (IIa-34), hydrochloride; MS: 262 [M+H⁺],
1-{1-[3-(5-fluoro-1H-indol-3-yl)propyl]pyrrolidin-3-yl}methaneamine (IIa-35), hydrochloride; MS: 276 [M+H⁺],
1-{1-[3-(5-chloro-1H-indol-3-yl)propyl]azetidin-3-yl}methaneamine (IIa-36), hydrochloride; MS: 278 [M+H⁺],
1-{1-[2-(2,3-dihydro-1,4-benzodioxan-5-yloxy)ethyl]azetidin-3-yl}methaneamine (IIa-37), MS: 275 [M+H⁺],
1-{1-[2-(2,3-dihydro-1,4-benzodioxan-5-yloxy)ethyl]pyrrolidin-3-yl}methaneamine (IIa-38), MS: 289 [M+H⁺].

### EXAMPLE 2

### Preparation of Compounds (I) According to the Invention

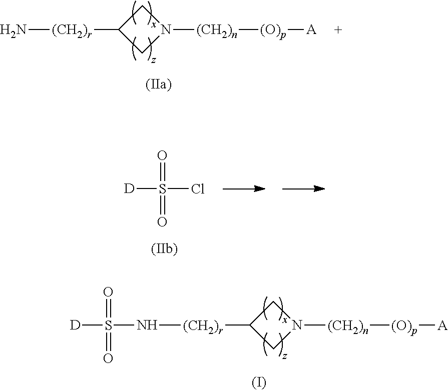

Depending on the type and form of the starting amine (IIa), the compounds (I) according to the invention were prepared using one of the three following procedures.

### 2a) Procedure for Starting Amines (IIa) as Hydrochlorides

To a solution of amine (IIa) hydrochloride (0.6 mmol) in methylene chloride cesium carbonate (1.2 mmol), the appropriate sulphonyl chloride (IIb) and DMAP (0.12 mmol) were added. The mixture was stirred overnight at room temperature, then inorganic solid was filtered off and from the filtrate solvent was evaporated under reduced pressure. Residue was purified by column chromatography on silica gel with a solvent system methylene chloride/methanol 95:5 v/v as eluent, to afford compound (I).

### 2b) Procedure for Starting Amines (IIa) as Trifluoroacetates

To amine (IIa) trifluoroacetate (0.5 mmol) 10 ml of dry N,N-dimethylformamide (10 ml), DIPEA (1 ml) and sulphonyl chloride (IIb) (0.6 mmol) in one portion were added. The mixture was stirred overnight at room temperature. Then saturated aqueous sodium bicarbonate solution was added to the mixture and the whole was extracted with ethyl acetate. The organic layer was washed with brine, dried over anhydrous magnesium sulphate, and subsequently the solvent was evaporated under reduced pressure. Residue was purified by column chromatography on silica gel using a solvent system methylene chloride/methanol 100:0-90:10 v/v as eluent to obtain compound (I).

### 2c) the Procedure for Starting Amines (IIa) as Free Bases

To amine (IIa) (0.4 mmol) dry methylene chloride (10 ml), pyridine (1 ml) and sulphonyl chloride (IIb) (0.4 mmol) in one portion were added. The mixture was stirred overnight at room temperature. Then, after addition of small amount of toluene, pyridine was evaporated under reduced pressure, and the residue was extracted using solvent system system water/ethyl acetate. The organic layer was dried over anhydrous magnesium sulphate and after evaporation of the solvent, the residue was purified by column chromatography on silica get using a solvent system methylene chloride/methanol 100:0-90:10 v/v as eluent to obtain compound (I).

Structures of compounds (I) according to the invention were confirmed by MS and/or ¹H NMR.

Yields of compounds (I) were in the range of 65-90%, and HPLC purities thereof in the range of 90-100%.

According to the above procedures, the following compounds (I) of the invention were prepared.

As starting materials commercially available sulphonyl chlorides (IIb) were used:
benzenesulphonyl chloride (IIb-1),
3-fluorobenzenesulphonyl chloride (IIb-2),
4-fluorobenzenesulphonyl chloride (IIb-3),
3-chlorobenzenesulphonyl chloride (IIIb-4),
4-chlorobenzenesulphonyl chloride (IIb-5),
4-bromobenzenesulphonyl chloride (IIb-6),
3-chloro-4-fluoro-benzenesulphonyl chloride (IIb-7),
3-methylbenzenesulphonyl chloride (IIb-8),
4-propylbenzenesulphonyl chloride (IIb-9),
4-tert-butylbenzenesulphonyl chloride (IIb-10),
3-(trifluoromethyl)benzenesulphonyl chloride (IIb-11),
4(trifluoromethyl)benzenesulphonyl chloride (IIb-12),
3-methoxybenzenesulphonyl chloride (IIb-13),
3-hydroxybenzenesulphonyl chloride (IIb-14),
3-cyanobenzenesulphonyl chloride (IIb-15),
4-cyanobenzenesulphonyl chloride (IIb-16),
naphthalene-1-sulphonyl chloride (IIb-17),
naphthalene-2-sulphonyl chloride (IIb-18),
6-chloronaphthalene-2-sulphonyl chloride (IIb-19),
5-chlorothiophene-2-sulphonyl chloride (IIb-20),
2,3-dihydrobenzofuran-6-sulphonyl chloride (IIb-21),
benzothiophene-2-sulphonyl chloride (IIb-22),
benzothiophene-3-sulphonyl chloride (IIb-23),
6-chlorobenzothiophene-2-sulphonyl chloride (IIb-24),
5-fluoro-3-methyl-benzothiophene-2-sulphonyl chloride (IIb-25),
5-chloro-3-methyl-benzothiophene-2-sulphonyl chloride (IIb-26),
imidazo[1,2-a]pyridine-3-sulphonyl chloride (IIb-27),
1,3-benzothiazole-4-sulphonyl chloride (IIb-28),
3-bromobenzenesulphonyl chloride (IIb-29),
4-iodobenzenesulphonyl chloride (IIb-30),
3,4-difluorobenzenesulphonyl chloride (IIb-31),
3,4-dichlorobenzenesulphonyl chloride (IIb-32),
4-methylbenzenesulphonyl chloride (IIb-33),
4-methoxybenzenesulphonyl chloride (IIb-34),
4-(trifluoromethoxy)benzenesulphonyl chloride (IIb-35),
biphenyl-4-sulphonyl chloride (IIb-36),
6-chloronaphthalene-2-sulphonyl chloride (IIb-37),
7-chloronaphthalene-2-sulphonyl chloride (IIb-38),
6-methoxynaphthalene-2-sulphonyl chloride (IIb-39),
thiophene-2-sulphonyl chloride (IIb-40),
thiophene-3-sulphonyl chloride (IIb-41),
2,5-dimethylthiophene-3-sulphonyl chloride (IIb-42),
5-isoxazol-5-ylthiophene-2-sulphonyl chloride (IIb-43),
1-methyl-1H-imidazole-4-sulphonyl chloride (IIb-44),
5-methylisoxazole-4-sulphonyl chloride (IIb-45),
1,3-thiazole-2-sulphonyl chloride (IIb-46),
2-oxo-2,3-dihydro-1H-indole-5-sulphonyl chloride (IIb-47),
1,3-benzodioxole-5-sulphonyl chloride (IIb-48),
1-methyl-1H-indole-4-sulphonyl chloride (IIb-50),
1-methyl-1H-indole-5-sulphonyl chloride (IIb-51),
1-benzofuran-2-sulphonyl chloride (IIb-52),
6-fluoro-1-benzothiophene-2-sulphonyl chloride (IIb-53),
5-methyl-1-benzothiophene-2-sulphonyl chloride (IIb-54),
1,3-benzothiazole-5-sulphonyl chloride (IIb-55),
   and the appropriate amines (IIa), as described above.

According to the above procedures the following compounds (I) of the invention were prepared.

### Compound 1. N-[1-[3-(6-Fluoro-1,2-benzoxazol-3-yl)propyl]azetidin-3-yl]benzene-sulphonamide

The title compound was prepared starting from amine (IIa-1) and sulphonyl chloride (IIb-1). MS: 390 [M+H⁺]

### Compound 2. 3-Fluoro-N-[1-[3-(6-fluoro-1,2-benzoxazol-3-yl)propyl]azetidin-3-yl]-benzenesulphonamide

The title compound was prepared starting from amine (IIa-1) and sulphonyl chloride (IIb-2). MS: 408 [M+H⁺]

### Compound 3. 4-Fluoro-N-[1-[3-(6-fluoro-1,2-benzoxazol-3-yl)propyl]azetidin-3-yl]benzene-sulphonamide

The title compound was prepared starting from amine (IIa-1) and sulphonyl chloride (IIb-3). MS: 408 [M+H⁺]

### Compound 4. 3-Chloro-N-[1-[3-(6-fluoro-1,2-benzoxazol-3-yl)propyl]azetidin-3-yl]benzene-sulphonamide

The title compound was prepared starting from amine (IIa-1) and sulphonyl chloride (IIIb-4). MS: 424 [M+H⁺]

### Compound 5. N-[1-[3-(6-Fluoro-1,2-benzoxazol-3-yl)propyl]azetidin-3-yl]-3-methyl-benzene-sulphonamide

The title compound was prepared starting from amine (IIa-1) and sulphonyl chloride (IIb-8). MS: 404 [M+H⁺]

### Compound 6. N-[1-[3-(6-Fluoro-1,2-benzoxazol-3-yl)propyl]pyrrolidin-3-yl]benzene-sulphonamide

The title compound was prepared starting from amine (IIIa-2) and sulphonyl chloride (IIb-1).

¹H-NMR (300 MHz, CDCl₃): 7.96-7.88 (m, 2H), 7.60-7.43 (m, 4H), 7.21-7.18 (m, 1H), 7.08-7.01 (m, 1H), 3.88-3.82 (m, 1H), 3.01-2.96 (m, 2H), 2.82-2.77 (m, 1H), 2.50-2.38 (m, 3H), 2.20-1.87 (m, 4H), 1.62-1.52 (m, 2H); MS: 444 [M+H⁺].

### Compound 7. 3-Fluoro-N-[1-[3-(6-fluoro-1,2-benzoxazol-3-yl)propyl]pyrrolidin-3-yl]-benzenesulphonamide

The title compound was prepared starting from amine (IIa-2) and sulphonyl chloride (IIb-2).

¹H-NMR (300 MHz, CDCl₃): 7.78-7.461 (m, 4H), 7.18-7.11 (m, 2H), 7.08-7.00 (m, 1H), 3.98-3.82 (m, 1H), 3.02-2.95 (m, 2H), 2.80-2.78 (m, 1H), 2.44-2.37 (m, 3H), 2.21-1.95 (m, 4H), 1.60-1.52 (m, 2H); MS: 422 [M+H⁺].

### Compound 8. 4-Fluoro-N-[1-[3-(6-fluoro-1,2-benzoxazol-3-yl)propyl]pyrrolidin-3-yl]benzenesulphonamide

The title compound was prepared starting from amine (IIa-2) and sulphonyl chloride (IIb-3).

¹H-NMR (300 MHz, CDCl₃): 7.98-7.82 (m, 2H), 7.61-7.58 (m, 1H), 7.20-7.16 (m, 3H), 7.08-7.00 (m, 1H), 3.82-3.78 (m, 1H), 3.00-2.83 (m, 2H), 2.80-2.72 (m, 1H), 2.45-2.28 (m, 3H), 2.20-1.96 (m, 4H), 1.52-1.40 (m, 2H); MS: 422 [M+H⁺].

### Compound 9. 3-Chloro-N-[1-[3-(6-fluoro-1,2-benzoxazol-3-yl)propyl]pyrrolidin-3-yl]benzenesulphonamide

The title compound was prepared starting from amine (IIa-2) and sulphonyl chloride (IIb-4).

¹H-NMR (300 MHz, CDCl₃): 7.82-7.78 (m, 1H), 7.75-7.70 (d, 1H, J=7.9 Hz), 7.60-7.52 (m, 3H), 7.21-7.19 (m, 1H), 7.06-7.01 (m, 1H), 3.85-3.80 (m, 1H), 3.00-2.96 (m, 2H), 2.80-2.76 (m, 1H), 2.52-2.38 (m, 3H), 2.20-1.85 (m, 4H), 1.58-1.43 (m, 2H); MS: 438[M+H⁺].

### Compound 10. 4-Bromo-N-[1-[3-(6-fluoro-1,2-benzoxazol-3-yl)propyl]pyrrolidin-3-yl]benzenesulphonamide

The title compound was prepared starting from amine (IIa-2) and sulphonyl chloride (IIb-6).

¹H-NMR (300 MHz, CDCl₃): 7.78-7.72 (m, 2H), 7.62-7.58 (m, 3H), 7.21-7.18 (m, 1H), 7.08-7.01 (m, 1H), 3.83-3.80 (m, 1H), 3.00-2.95 (m, 2H), 2.80-2.75 (m, 1H), 2.52-2.43 (m, 2H), 2.30-2.28 (m, 1H), 2.20-1.83 (m, 4H), 1.58-1.50 (m, 2H); MS: 483 [M+H⁺].

### Compound 11. 4-Chloro-3-fluoro-N-[1-[3-(6-fluoro-1,2-benzoxazol-3-yl)propyl]-pyrrolidin-3-yl]benzene-sulphonamide

The title compound was prepared starting from amine (IIa-2) and sulphonyl chloride (IIb-7).

¹H-NMR (300 MHz, CDCl₃): 7.97-7.93 (m, 1H), 7.80-7.65 (m, 1H), 7.60-7.55 (m, 1H), 7.22-7.20 (m, 2H), 7.08-7.01 (m, 1H), 3.80-3.71 (m, 1H), 2.97-2.83 (m, 2H), 2.80-2.72 (m, 1H), 2.45-2.28 (m, 3H), 2.21-1.97 (m, 4H), 1.60-1.53 (m, 2H); MS: 456 [M+H⁺].

### Compound 12. N-[1-[3-(6-Fluoro-1,2-benzoxazol-3-yl)propyl]pyrrolidin-3-yl]-3-methyl-benzenesulphonamide

The title compound was prepared starting from amine (IIa-2) and sulphonyl chloride (IIb-8).

¹H-NMR (300 MHz, CDCl₃): 7.61-7.57 (m, 3H), 7.43-7.38 (m, 2H), 7.21-7.18 (m, 1H), 7.08-7.01 (m, 1H), 3.80-3.76 (m, 1H), 2.96-2.82 (m, 2H), 2.81 (s, 3H), 2.79-2.74 (m, 1H), 2.42-2.28 (m, 3H), 2.20-1.96 (m, 4H), 1.59-1.50 (m, 2H); MS: 418 [M+H⁺].

### Compound 13. N-[1-[3-(6-Fluoro-1,2-benzoxazol-3-yl)propyl]pyrrolidin-3-yl]-4-propyl-benzenesulphonamide

The title compound was prepared starting from amine (IIa-2) and sulphonyl chloride (IIb-9).

¹H-NMR (300 MHz, CDCl₃): 7.80 (d, 2H, J=7.9 Hz), 7.62-7.58 (m, 1H), 7.36 (d, 2H, J=7.9 Hz), 7.21-7.18 (m, 1H), 7.08-7.01 (m, 1H), 3.12-3.02 (m, 4H), 2.98-2.90 (m, 1H), 2.67-2.60 (m, 4H), 2.27-2.20 (m, H), 1.75-1.60 (m, 5H), 0.95 (t, 2H, J=3.4 Hz); MS: 446[M+H⁺].

### Compound 14. 4-tert-Butyl-N-[1-[3-(6-fluoro-1,2-benzoxazol-3-yl)propyl]pyrrolidin-3-yl]benzenesulphonamide

The title compound was prepared starting from amine (IIa-2) and sulphonyl chloride (IIb-10).

¹H-NMR (300 MHz, CDCl₃): 7.78-7.63 (m, 2H), 7.62-7.45 (m, 3H), 7.20-7.18 (m, 1H), 7.06-7.02 (m, 1H), 3.82-3.78 (m, 1H), 2.98-2.92 (m, 2H), 2.80-2.75 (m, 1H), 2.55-2.42 (m, 3H), 1.98-1.92 (m, 4H), 1.58-1.48 (m, 2H), 1.32 (s, 9H); 4260[M+H⁺].

### Compound 15. N-[1-[3-(6-Fluoro-1,2-benzoxazol-3-yl)propyl]pyrrolidin-3-yl]-3-(trifluoromethyl)benzenesulphonamide

The title compound was prepared starting from amine (IIa-2) and sulphonyl chloride (IIb-11).

¹H-NMR (300 MHz, CDCl₃): 8.18-8.02 (m, 2H), 7.82 (d, 1H, J=7.9 Hz), 7.65-7.54 (m, 2H), 7.20 (t, 1H, J=7.4 Hz), 7.02 (t, 1H, J=7.9 Hz), 3.84-3.80 (m, 1H), 2.98-2.90 (m, 2H), 2.80-2.75 (m, 1H), 2.52-2.38 (m, 3H), 2.20-1.85 (m, 4H), 1.60-1.65 (m, 2H); MS: 472[M+H⁺].

### Compound 16. N-[1-[3-(6-Fluoro-1,2-benzoxazol-3-yl)propyl]pyrrolidin-3-yl]-4-(trifluoromethyl)benzenesulphonamide

The title compound was prepared starting from amine (IIa-2) and sulphonyl chloride (IIb-12).

¹H-NMR (300 MHz, CDCl₃): 7.98 (d, 2H, J=7.9 Hz), 7.78 (d, 2H, J=7.9 Hz), 7.60-7.57 (m, 1H), 7.21-7.19 (m, 1H), 7.07-7.01 (m, 1H), 3.95-3.92 (m, 1H), 2.98-2.92 (m, 2H), 2.90-2.87 (m, 1H), 2.55-2.38 (m, 3H), 2.20-1.85 (m, 4H), 1.60-1.52 (m, 2H); MS: 472 [M+H⁺].

### Compound 17. N-[1-[3-(6-Fluoro-1,2-benzoxazol-3-yl)propyl]pyrrolidin-3-yl]-3-methoxybenzenesulphonamide

The title compound was prepared starting from amine (IIa-2) and sulphonyl chloride (IIb-13).

¹H-NMR (300 MHz, CDCl₃): 7.61-7.57 (m, 1H), 7.53-7.40 (m, 3H), 7.21-7.18 (m, 1H), 7.08-7.00 (m, 1H), 3.81 (s, 3H), 3.83-3.78 (m, 1H), 2.98-2.82 (m, 2H), 2.80-2.74 (m, 1H), 2.43-2.28 (m, 3H), 2.20-1.96 (m, 4H), 1.58-1.50 (m, 2H); MS: 435 [M+H⁺].

### Compound 18. N-[1-[3-(6-Fluoro-1,2-benzoxazol-3-yl)propyl]pyrrolidin-3-yl]-3-hydroxybenzenesulphonamide

The title compound was prepared starting from amine (IIa-2) and sulphonyl chloride (IIb-14).

¹H-NMR (300 MHz, CDCl₃): 7.58-7.50 (m, 1H), 7.38-7.20 (m, 4H), 7.04-6.97 (m, 2H), 5.31 (s, 1H), 3.82-3.78 (m, 1H), 2.97-2.82 (m, 2H), 2.81-2.74 (m, 1H), 2.42-2.28 (m, 3H), 2.21-1.96 (m, 4H), 1.61-1.56 (m, 2H); MS: 420 [M+H⁺].

### Compound 19. 3-Cyano-N-[1-[3-(6-fluoro-1,2-benzoxazol-3-yl)propyl]pyrrolidin-3-yl]benzenesulphonamide

The title compound was prepared starting from amine (IIa-2) and sulphonyl chloride (IIb-15).

¹H-NMR (300 MHz, CDCl₃): 8.20-8.17 (m, 1H), 8.14-7.98 (m, 1H), 7.87-7.82 (m, 1H), 7.68-7.56 (m, 2H), 7.26-7.22 (m, 1H), 7.10-7.02 (m, 1H), 3.90-3.80 (s, 1H), 3.02-2.94 (m, 2H), 2.84-2.78 (m, 1H), 2.54-2.42 (m, 2H), 2.40-2.32 (m, 1H), 2.20-1.90 (m, 4H), 1.60-1.56 (m, 2H), MS: 429 [M+H⁺].

### Compound 20. N-[1-[3-(6-Fluoro-1,2-benzoxazol-3-yl)propyl]pyrrolidin-3-yl]naphthalene-1-sulphonamide

The title compound was prepared starting from amine (IIa-2) and sulphonyl chloride (IIb-17). MS: 454 [M+H⁺].

### Compound 21. N-[1-[3-(6-Fluoro-1,2-benzoxazol-3-yl)propyl]pyrrolidin-3-yl]naphthalene-2-sulphonamide

The title compound was prepared starting from amine (IIa-2) and sulphonyl chloride (IIb-18). MS: 454 [M+H⁺].

### Compound 22. 5-Chloro-N-[1-[3-(6-fluoro-1,2-benzoxazol-3-yl)propyl]pyrrolidin-3-yl]thiophene-2-sulphonamide

The title compound was prepared starting from amine (IIa-2) and sulphonyl chloride (IIb-20).

¹H-NMR (300 MHz, CDCl₃): 7.65-7.58 (m, 1H), 7.41 (d, 1H, J=2.9 Hz), 7.22-7.20 (m, 1H), 7.08-7.01 (m, 1H), 6.91 (d, 1H, J=2.9 Hz), 3.91-3.86 (m, 1H), 2.92-2.84 (m, 2H), 2.78-2.64 (m, 1H), 2.42-2.22 (m, 3H), 2.15-1.95 (m, 2H), 1.80-1.75 (m, 2H), 1.62-1.56 (m, 2H); MS: 444[M+H⁺].

### Compound 23. 6-Chloro-N-[1-[3-(6-fluoro-1,2-benzoxazol-3-yl)propyl]pyrrolidin-3-yl]naphthalene-2-sulphonamide

The title compound was prepared starting from amine (IIa-2) and sulphonyl chloride (IIb-19).

¹H-NMR (300 MHz, CDCl₃): 8.40 (s, 1H), 7.90-7.84 (m, 4H), 7.58-7.52 (m, 2H), 7.22-7.19 (m, 1/h), 6.98-6.90 (m, 1H), 3.89-3.82 (m, 1H), 2.98-2.92 (t, 2H, J=7.4 Hz), 2.78-2.70 (m, 1H), 2.50-2.40 (m, 2H), 2.38-2.32 (m, 1H), 2.18-1.84 (m, 4H), 1.58-1.48 (m, 2H); MS: 488 [M+H⁺].

### Compound 24. N-[1-[3-(6-Fluoro-1,2-benzoxazol-3-yl)propyl]pyrrolidin-3-yl]-2,3-dihydrobenzofuran-6-sulphonamide

The title compound was prepared starting from amine (IIa-2) and sulphonyl chloride (IIb-21).

¹H-NMR (300 MHz, CDCl₃): 7.60-7.52 (m, 3H), 7.21-7.18 (m, 1H), 7.08-7.00 (m, 1H), 6.75 (d, 1H, J=8.4 Hz), 4.70-4.60 (t, 2H, J=8.9 Hz), 3.80-3.74 (m, 1H), 3.28-3.18 (t, 2H, J=8.9 Hz), 2.98-2.92 (t, 2H, J=7.4 Hz), 2.74-2.64 (m, 1H), 2.50-2.38 (m, 3H), 2.26-2.16 (m, 1H), 2.10-1.87 (m, 3H), 1.60-1.48 (m, 2H), MS: 446 [M+H⁺].

### Compound 25. N-[1-[3-(6-Fluoro-1,2-benzoxazol-3-yl)propyl]pyrrolidin-3-yl]-benzothiophene-2-sulphonamide

The title compound was prepared starting from amine (IIIa-2) and sulphonyl chloride (IIb-22).

¹H-NMR (300 MHz, CDCl₃): 7.88-7.80 (m, 3H), 7.60-7.54 (m, 1H), 7.50-7.40 (m, 2H), 7.22-7.20 (m, 1H), 7.08-7.00 (m, 1H), 3.90-3.82 (m, 1H), 2.93-2.82 (m, 2H), 2.77-2.63 (m, 1H), 2.43-2.22 (m, 3H), 2.19-1.95 (m, 2H), 1.81-1.77 (m, 2H), 1.55-1.43 (m, 2H); MS: 460[M+H⁺].

### Compound 26. N-[1-[3-(6-Fluoro-1,2-benzoxazol-3-yl)propyl]pyrrolidin-3-yl]-benzothiophene-3-sulphonamide

The title compound was prepared starting from amine (IIa-2) and sulphonyl chloride (IIb-23).

¹H-NMR (300 MHz, CDCl₃): 8.21 (s, 1H), 8.20-8.17 (d, 1H, J=7.4 Hz), 7.84-7.80 (d, 1H, J=7.4 Hz), 7.58-7.50 (m, 1H), 7.48-7.45 (m, 2H), 7.24-7.20 (m, 1H), 7.08-7.02 (m, 1H), 3.90-3.80 (m, 1H), 2.90-2.80 (m, 2H), 2.78-2.64 (m, 1H), 2.43-2.24 (m, 3H), 2.18-1.97 (m, 2H), 1.80-1.75 (m, 2H), 1.57-1.45 (m, 2H); MS: 460[M+H⁺].

### Compound 27. 6-Chloro-N-[1-[3-(6-fluoro-1,2-benzoxazol-3-yl)propyl]pyrrolidin-3-yl]benzothiophene-2-sulphonamide

The title compound was prepared starting from amine (IIa-2) and sulphonyl chloride (IIb-24).

¹H-NMR (300 MHz, CDCl₃): 7.84-7.78 (m, 3H), 7.60-7.56 (m, 1H), 7.39 (d, 1H, J=7.6 Hz), 7.20 (d, 1H, J=7.6 Hz), 7.06-7.00 (m, 1H), 3.98-3.82 (m, 1H), 3.00-2.94 (m, 2H), 2.82-2.76 (m, 1H), 2.44-2.35 (m, 3H), 2.20-1.90 (m, 4H), 1.62-1.54 (m, 2H); MS: 494 [M+H⁺].

### Compound 28. 5-Fluoro-N-[1-[3-(6-fluoro-1,2-benzoxazol-3-yl)propyl]pyrrolidin-3-yl]-3-methyl-benzothiophene-2-sulphonamide

The title compound was prepared starting from amine (IIa-2) and sulphonyl chloride (IIb-25).

¹H-NMR (300 MHz, CDCl₃): 7.78-7.70 (m, 1H), 7.60-7.54 (m, 1H), 7.44-7.41 (m, 1H), 7.24-7.19 (m, 2H), 7.08-7.02 (m, 1H), 4.04-3.98 (m, 1H), 3.01-2.97 (m, 2H), 2.94-2.87 (m, 1H), 2.63 (s, 3H), 2.42-2.35 (m, 3H), 2.21-1.91 (m, 4H), 1.61-1.56 (m, 2H); MS: 492 [M+H⁺].

### Compound 29. 5-Chloro-N-[1-[3-(6-fluoro-1,2-benzoxazol-3-yl)propyl]pyrrolidin-3-yl]-3-methyl-benzothiophene-2-sulphonamide

The title compound was prepared starting from amine (IIa-2) and sulphonyl chloride (IIb-26).

¹H-NMR (300 MHz, CDCl₃): 7.80-7.76 (m, 2H), 7.61-7.56 (m, 1H), 7.43-7.40 (m, 1H), 7.20-7.18 (m, 1H), 6.98-6.90 (m, 1H), 3.98-3.92 (m, 1H), 2.98-2.92 (t, 2H, J=7.4 Hz), 2.80-2.74 (m, 2H), 2.62 (s, 3H), 2.58-2.40 (m, 2H), 2.18-1.98 (m, 4H), 1.78-1.72 (m, 2H); MS: 510 [M+H⁺].

### Compound 30. N-[1-[3-(6-Fluoro-1,2-benzoxazol-3-yl)propyl]pyrrolidin-3-yl]imidazo-[1,2-a]pyridine-3-sulphonamide

The title compound was prepared starting from amine (IIIa-2) and sulphonyl chloride (IIb-27).

¹H-NMR (300 MHz, CDCl₃): 8.60 (m, 1H), 8.18 (s, 1H), 7.68-7.65 (m, 1H), 7.61-7.42 (m, 4H), 7.08-7.01 (m, 1H), 3.85-3.81 (m, 1H), 3.02-2.96 (m, 2H), 2.79-2.76 (m, 1H), 2.52-2.38 (m, 3H), 2.21-1.87 (m, 4H), 1.62-1.52 (m, 2H); MS: 444[M+H⁺].

### Compound 31. N-[1-[3-(6-Fluoro-1,2-benzoxazol-3-yl)propyl]pyrrolidin-3-yl]-1,3-benzothiazole-4-sulphonamide

The title compound was prepared starting from amine (IIa-2) and sulphonyl chloride (IIb-28).

¹H-NMR (300 MHz, CDCl₃): 9.20 (s, 1H), 8.20-8.15 (m, 2H), 7.60-7.55 (m, 2H), 7.20-7.18 (m, 1H), 7.08-7.01 (m, 1H), 3.87-3.80 (m, 1H), 3.00-2.96 (m, 2H), 2.82-2.77 (m, 1H), 2.50-2.38 (m, 3H), 2.20-1.87 (m, 4H), 1.62-1.52 (m, 2H); MS: 444[M+H⁺].

### Compound 32. N-[1-[3-(6-Fluoro-1,2-benzoxazol-3-yl)propyl]-4-piperidine]benzene-sulphonamide

The title compound was prepared starting from amine (IIIa-3) and sulphonyl chloride (IIb-1). MS: 418 [M+H⁺].

### Compound 33. N-[1-[3-(6-Fluoro-1,2-benzoxazol-3-yl)propyl]-4-piperidine]-3-methyl-benzenesulphonamide

The title compound was prepared starting from amine (IIIa-3) and sulphonyl chloride (IIb-8). MS: 432 [M+H⁺].

### Compound 34. N-[1-[3-(6-Fluoro-1,2-benzoxazol-3-yl)propyl]-4-piperidine]-naphthalene-1-sulphonamide

The title compound was prepared starting from amine (IIIa-3) and sulphonyl chloride (IIb-17). MS: 468 [M+H⁺].

### Compound 35. N-[1-[3-(6-Fluoro-1,2-benzoxazol-3-yl)propyl]-4-piperidine]-naphthalene-2-sulphonamide

The title compound was prepared starting from amine (IIa-3) and sulphonyl chloride (IIb-18). MS: 468 [M+H⁺].

### Compound 36. N-[[1-[3-(6-Fluoro-1,2-benzoxazol-3-yl)propyl]-4-piperidine]methyl]-3-methylbenzenesulphonamide

The title compound was prepared starting from amine (IIa-4) and sulphonyl chloride (IIb-8). MS: 446 [M+H⁺].

### Compound 37. N-[[1-[3-(6-Fluoro-1,2-benzoxazol-3-yl)propyl]-4-piperidine]methyl]-naphthalene-1-sulphonamide

The title compound was prepared starting from amine (IIa-4) and sulphonyl chloride (IIb-17). MS: 482 [M+H⁺].

### Compound 38. N-[[1-[3-(6-Fluoro-1,2-benzoxazol-3-yl)propyl]-4-piperidine]methyl]-naphthalene-2-sulphonamide

The title compound was prepared starting from amine (IIa-4) and sulphonyl chloride (IIb-18). MS: 482 [M+H⁺].

### Compound 39. N-[1-[2-(1,2-Benzothiazol-3-yloxy)ethyl]pyrrolidin-3-yl]naphthalene-2-sulphonamide

The title compound was prepared starting from amine (IIa-5) and sulphonyl chloride (IIb-18). MS: 454 [M+H⁺].

### Compound 40. N-[1-[2-(1,2-Benzothiazol-3-yloxy)ethyl]-4-piperidine]naphthalene-2-sulphonamide

The title compound was prepared starting from amine (IIa-6) and sulphonyl chloride (IIb-18). MS: 468 [M+H⁺].

### Compound 41. N-[[1-[2-(1,2-Benzothiazol-3-yloxy)ethyl]azetidin-3-yl]methyl]-3-hydroxybenzenesulphonamide

The title compound was prepared starting from amine (IIa-7) and sulphonyl chloride (IIb-14). MS: 420 [M+H⁺].

### Compound 42

### N-[[1-[2-(1,2-Benzothiazol-3-yloxy)ethyl]azetidin-3-yl]methyl]naphthalene-2-sulphonamide

The title compound was prepared starting from amine (IIa-7) and sulphonyl chloride (IIb-18). MS: 454 [M+H⁺].

### Compound 43. N-[[1-[2-(1,2-Benzothiazol-3-yloxy)ethyl]pyrrolidin-3-yl]methyl]-3-hydroxy-benzenesulphonamide

The title compound was prepared starting from amine (IIa-8) and sulphonyl chloride (IIb-14). MS: 434 [M+H⁺].

### Compound 44. N-[[1-[2-(1,2-Benzothiazol-3-yloxy)ethyl]pyrrolidin-3-yl]methyl]naphthalene-2-sulphonamide

The title compound was prepared starting from amine (IIa-8) and sulphonyl chloride (IIb-18). MS: 468 [M+H⁺].

### Compound 45

### N-[1-[2-(1H-indol-4-yloxy)ethyl]azetidin-3-yl]benzenesulphonamide

The title compound was prepared starting from amine (IIa-9) and sulphonyl chloride (IIb-1). MS: 372 [M+H⁺]

### Compound 46. 4-Fluoro-N-[1-[2-(1H-indol-4-yloxy)ethyl]azetidin-3-yl]benzene-sulphonamide

The title compound was prepared starting from amine (IIa-9) and sulphonyl chloride (IIb-3). MS: 390 [M+H⁺]

### Compound 47. 3-Chloro-N-[1-[2-(1H-indol-4-yloxy)ethyl]azetidin-3-yl]benzene-sulphonamide

The title compound was prepared starting from amine (IIa-9) and sulphonyl chloride (IIb-4). MS: 406 [M+H⁺]

### Compound 48. N-[1-[2-(1H-Indol-4-yloxy)ethyl]azetidin-3-yl]-3-methyl-benzenesulphonamide

The title compound was prepared starting from amine (IIa-9) and sulphonyl chloride (IIb-8). MS: 386 [M+H⁺]

### Compound 49. N-[1-[2-(1H-Indol-4-yloxy)ethyl]azetidin-3-yl]naphthalene-1-sulphonamide

The title compound was prepared starting from amine (IIIa-9) and sulphonyl chloride (IIb-17). MS: 422 [M+H⁺]

### Compound 50. N-[1-[2-(1H-Indol-4-yloxy)ethyl]azetidin-3-yl]naphthalene-2-sulphonamide

The title compound was prepared starting from amine (IIIa-9) and sulphonyl chloride (IIb-18). MS: 422 [M+H⁺]

### Compound 51. N-[1-[2-(1H-Indol-4-yloxy)ethyl]pyrrolidin-3-yl]benzenesulphonamide

The title compound was prepared starting from amine (IIIa-10) and sulphonyl chloride (IIb-1). MS: 386 [M+H⁺]

### Compound 52. N-[1-[2-(1H-Indol-4-yloxy)ethyl]pyrrolidin-3-yl]-3-methylbenzene-sulphonamide

The title compound was prepared starting from amine (IIa-10) and sulphonyl chloride (IIb-8). MS: 400 [M+H⁺]

### Compound 53. N-[1-[2-(1H-indol-4-yloxy)ethyl]pyrrolidin-3-yl]naphthalene-1-sulphonamide

The title compound was prepared starting from amine (IIa-10) and sulphonyl chloride (IIb-17). MS: 436 [M+H⁺]

### Compound 54. N-[1-[2-(1H-Indol-4-yloxy)ethyl]pyrrolidin-3-yl]naphthalene-2-sulphonamide

The title compound was prepared starting from amine (IIa-10) and sulphonyl chloride (IIb-18). MS: 436 [M+H⁺]

### Compound 55. N-[1-[2-(1H-Indol-4-yloxy)ethyl]-4-piperidine]benzenesulphonamide

The title compound was prepared starting from amine (IIa-11) and sulphonyl chloride (IIb-1). MS: 400 [M+H⁺]

### Compound 56. N-[1-[2-(1H-Indol-4-yloxy)ethyl]-4-piperidine]-3-methylbenzene-sulphonamide

The title compound was prepared starting from amine (IIa-11) and sulphonyl chloride (IIb-8). MS: 414 [M+H⁺]

### Compound 57. 4-tert-Butylo-N-[1-[2-(1H-indol-4-yloxy)ethyl]-4-piperidine]benzene-sulphonamide

The title compound was prepared starting from amine (IIa-11) and sulphonyl chloride (IIb-10). MS: 456 [M+H⁺]

### Compound 58. N-[1-[2-(1H-indol-4-yloxy)ethyl]-4-piperidine]-4-(trifluoromethyl)-benzene-sulphonamide

The title compound was prepared starting from amine (IIa-11) and sulphonyl chloride (IIb-12). MS: 468 [M+H⁺]

### Compound 59. 4-Cyano-N-[1-[2-(1H-indol-4-yloxy)ethyl]-4-piperidine]benzene-sulphonamide

The title compound was prepared starting from amine (IIa-11) and sulphonyl chloride (IIb-16). MS: 425 [M+H⁺]

### Compound 60. N-[1-[2-(1H-Indol-4-yloxy)ethyl]-4-piperidine]naphthalene-1-sulphonamide

The title compound was prepared starting from amine (IIa-11) and sulphonyl chloride (IIb-17). MS: 450 [M+H⁺]

### Compound 61. N-[1-[2-(1H-Indol-4-yloxy)ethyl]-4-piperidine]naphthalene-2-sulphonamide

The title compound was prepared starting from amine (IIa-11) and sulphonyl chloride (IIb-18). MS: 450 [M+H⁺]

### Compound 62. 5-Chloro-N-[1-[2-(1H-indol-4-yloxy)ethyl]-4-piperidine]-3-methyl-benzothiophene-2-sulphonamide

The title compound was prepared starting from amine (IIa-11) and sulphonyl chloride (IIb-26). MS: 504 [M+H⁺]

### Compound 63. N-[[1-[2-(1H-Indol-4-yloxy)ethyl]-4-piperidine]methyl]benzene-sulphonamide

The title compound was prepared starting from amine (IIa-12) and sulphonyl chloride (IIb-1). MS: 414 [M+H⁺]

### Compound 64. N-[[1-[2-(1H-Indol-4-yloxy)ethyl]-4-piperidine]methyl]-3-methyl-benzenesulphonamide

The title compound was prepared starting from amine (IIa-12) and sulphonyl chloride (IIb-8). MS: 428 [M+H⁺]

### Compound 65. 3-Hydroxy-N-[[1-[2-(1H-indol-4-yloxy)ethyl]-4-piperidine]methyl]-benzenesulphonamide

The title compound was prepared starting from amine (IIa-12) and sulphonyl chloride (IIb-14). MS: 430 [M+H⁺]

### Compound 66. N-[[1-[2-(1H-Indo-4-yloxy)ethyl]-4-piperidine]methyl]naphthalene-1-sulphonamide

The title compound was prepared starting from amine (IIa-12) and sulphonyl chloride (IIIb-17). MS: 464 [M+H⁺]

### Compound 67. N-[[1-[2-(1H-Indol-4-yloxy)ethyl]-4-piperidine]methyl]naphthalene-2-sulphonamide

The title compound was prepared starting from amine (IIa-12) and sulphonyl chloride (IIb-18). MS: 464 [M+H⁺]

### Compound 68. N-[1-[2-(1H-Indol-6-yloxy)ethyl]pyrrolidin-3-yl]benzenesulphonamide

The title compound was prepared starting from amine (IIa-13) and sulphonyl chloride (IIb-1). MS: 386 [M+H⁺]

### Compound 69. N-[1-[2-(1H-Indol-6-yloxy)ethyl]pyrrolidin-3-yl]-3-methylbenzene-sulphonamide

The title compound was prepared starting from amine (IIa-13) and sulphonyl chloride (IIb-8). MS: 400 [M+H⁺]

### Compound 70. N-[[1-[2-(1H-Indol-6-yloxy)ethyl]-4-piperidine]methyl]benzene-sulphonamide

The title compound was prepared starting from amine (IIa-14) and sulphonyl chloride (IIb-1). MS: 414 [M+H⁺]

### Compound 71. N-[[1-[2-(1H-Indol-6-yloxy)ethyl]-4-piperidine]methyl]naphthalene-1-sulphonamide

The title compound was prepared starting from amine (IIa-14) and sulphonyl chloride (IIb-17). MS: 464 [M+H⁺]

### Compound 72. N-[[1-[2-(1H-Indol-6-yloxy)ethyl]-4-piperidine]methyl]naphthalene-2-sulphonamide

The title compound was prepared starting from amine (IIa-14) and sulphonyl chloride (IIb-18). MS: 464 [M+H⁺]

### Compound 73. N-[1-[3-(5-Fluoro-1H-indol-3-yl)propyl]pyrrolidin-3-yl]benzene-sulphonamide

The title compound was prepared starting from amine (IIa-15) and sulphonyl chloride (IIb-1). MS: 402 [M+H⁺]

### Compound 74. 3-Fluoro-N-[1-[3-(5-fluoro-1H-indol-3-yl)propyl]pyrrolidin-3-yl]-benzenesulphonamide

The title compound was prepared starting from amine (IIa-15) and sulphonyl chloride (IIb-2). MS: 420 [M+H⁺]

### Compound 75. 4-Fluoro-N-[1-[3-(5-fluoro-1H-indol-3-yl)propyl]pyrrolidin-3-yl]benzene-sulphonamide

The title compound was prepared starting from amine (IIa-15) and sulphonyl chloride (IIb-3). MS: 420 [M+H⁺]

### Compound 76. 3-Chloro-N-[1-[3-(5-fluoro-1H-indol-3-yl)propyl]pyrrolidin-3-yl]-benzenesulphonamide

The title compound was prepared starting from amine (IIa-15) and sulphonyl chloride (IIb-4). MS: 436 [M+H⁺]

### Compound 77. 4-Chloro-N-[1-[3-(5-fluoro-1H-indol-3-yl)propyl]pyrrolidin-3-yl]-benzenesulphonamide

The title compound was prepared starting from amine (IIa-15) and sulphonyl chloride (IIb-5). MS: 436 [M+H⁺]

### Compound 78. N-[1-[3-(5-Fluoro-1H-indol-3-yl)propyl]pyrrolidin-3-yl]-3-methyl-benzenesulphonamide

The title compound was prepared starting from amine (IIa-15) and sulphonyl chloride (IIb-8). MS: 416 [M+H⁺]

### Compound 79. N-[1-[3-(5-Fluoro-1H-indol-3-yl)propyl]pyrrolidin-3-yl]naphthalene-1-sulphonamide

The title compound was prepared starting from amine (IIa-15) and sulphonyl chloride (IIb-17). MS: 452 [M+H⁺]

### Compound 80. N-[1-[3-(5-Fluoro-1H-indol-3-yl)propyl]pyrrolidin-3-yl]naphthalene-2-sulphonamide

The title compound was prepared starting from amine (IIa-15) and sulphonyl chloride (IIb-18). MS: 452 [M+H⁺]

### Compound 81. N-[1-[3-(5-Chloro-1H-indol-3-yl)propyl]pyrrolidin-3-yl]benzene-sulphonamide

The title compound was prepared starting from amine (IIa-16) and sulphonyl chloride (IIb-1). MS: 418 [M+H⁺]

### Compound 82. N-[1-[3-(5-Chloro-1H-indol-3-yl)propyl]pyrrolidin-3-yl]-3-fluoro-benzene-sulphonamide

The title compound was prepared starting from amine (IIa-16) and sulphonyl chloride (IIb-2). MS: 436 [M+H⁺]

### Compound 83. N-[1-[3-(5-Chloro-1H-indol-3-yl)propyl]pyrrolidin-3-yl]-4-fluoro-benzenesulphonamide

The title compound was prepared starting from amine (IIa-16) and sulphonyl chloride (IIb-3). MS: 436 [M+H⁺]

### Compound 84. 3-Chloro-N-[1-[3-(5-chloro-1H-indol-3-yl)propyl]pyrrolidin-3-yl]benzenesulphonamide

The title compound was prepared starting from amine (IIa-16) and sulphonyl chloride (IIb-4). MS: 452 [M+H⁺]

### Compound 85. 4-Chloro-N-[1-[3-(5-chloro-1H-indol-3-yl)propyl]pyrrolidin-3-yl]-benzene-sulphonamide

The title compound was prepared starting from amine (IIa-16) and sulphonyl chloride (IIIb-5). MS: 452 [M+H⁺]

### Compound 86

### N-[1-[3-(5-chloro-1H-indol-3-yl)propyl]pyrrolidin-3-yl]-3-methyl-benzenesulphonamide

The title compound was prepared starting from amine (IIa-16) and sulphonyl chloride (IIb-8). MS: 432 [M+H⁺]

### Compound 87. N-[1-[3-(5-Chloro-1H-indol-3-yl)propyl]pyrrolidin-3-yl]naphthalene-2-sulphonamide

The title compound was prepared starting from amine (IIa-16) and sulphonyl chloride (IIb-18). MS: 468 [M+H⁺]

### Compound 88. N-[1-[2-(2,3-Dihydro-1,4-benzodioxan-8-yloxy)ethyl]azetidin-3-yl]-benzenesulphonamide

The title compound was prepared starting from amine (IIa-17) and sulphonyl chloride (IIb-1). MS: 391 [M+H⁺]

### Compound 89. N-[1-[2-(2,3-Dihydro-1,4-benzodioxan-8-yloxy)ethyl]azetidin-3-yl]-3-fluoro-benzenesulphonamide

The title compound was prepared starting from amine (IIa-17) and sulphonyl chloride (IIb-2). MS: 409 [M+H⁺]

### Compound 90. N-[1-[2-(2,3-Dihydro-1,4-benzodioxan-8-yloxy)ethyl]azetidin-3-yl]-4-fluoro-benzenesulphonamide

The title compound was prepared starting from amine (IIa-17) and sulphonyl chloride (IIb-3). MS: 409 [M+H⁺]

### Compound 91. 3-Chloro-N-[1-[2-(2,3-dihydro-1,4-benzodioxan-8-yloxy)ethyl]azetidin-3-yl]benzenesulphonamide

The title compound was prepared starting from amine (IIIa-17) and sulphonyl chloride (IIb-4). MS: 425 [M+H⁺]

### Compound 92. 4-Chloro-N-[1-[2-(2,3-dihydro-1,4-benzodioxan-8-yloxy)ethyl]azetidin-3-yl]benzene-sulphonamide

The title compound was prepared starting from amine (IIa-17) and sulphonyl chloride (IIb-5). MS: 425 [M+H⁺]

### Compound 93. N-[1-[2-(2,3-Dihydro-1,4-benzodioxan-8-yloxy)ethyl]azetidin-3-yl]-3-methyl-benzenesulphonamide

The title compound was prepared starting from amine (IIa-17) and sulphonyl chloride (IIb-8). MS: 405 [M+H⁺]

### Compound 94. N-[1-[2-(2,3-Dihydro-1,4-benzodioxan-8-yloxy)ethyl]azetidin-3-yl]-naphthalene-1-sulphonamide

The title compound was prepared starting from amine (IIa-17) and sulphonyl chloride (IIb-17). MS: 441 [M+H⁺]

### Compound 95. N-[1-[2-(2,3-Dihydro-1,4-benzodioxan-8-yloxy)ethyl]azetidin-3-yl]naphthalene-2-sulphonamide

The title compound was prepared starting from amine (IIa-17) and sulphonyl chloride (IIb-18). MS: 441 [M+H⁺]

### Compound 96. N-[1-[2-(2,3-Dihydro-1,4-benzodioxan-8-yloxy)ethyl]pyrrolidin-3-yl]-benzenesulphonamide

The title compound was prepared starting from amine (IIIa-18) and sulphonyl chloride (IIb-1). MS: 405 [M+H⁺]

### Compound 97. N-[1-[2-(2,3-Dihydro-1,4-benzodioxan-8-yloxy)ethyl]pyrrolidin-3-yl]-3-methylbenzenesulphonamide

The title compound was prepared starting from amine (IIa-18) and sulphonyl chloride (IIb-8). MS: 419 [M+H⁺]

### Compound 98. N-[1-[2-(2,3-Dihydro-1,4-benzodioxan-8-yloxy)ethyl]pyrrolidin-3-yl]-naphthalene-1-sulphonamide

The title compound was prepared starting from amine (IIa-18) and sulphonyl chloride (IIb-17). MS: 457 [M+H⁺]

### Compound 99. N-[1-[2-(2,3-Dihydro-1,4-benzodioxan-8-yloxy)ethyl]pyrrolidin-3-yl]-naphthalene-2-sulphonamide

The title compound was prepared starting from amine (IIa-18) and sulphonyl chloride (IIb-18). MS: 457 [M+H⁺]

### Compound 100. N-[1-[2-(2,3-dihydro-1,4-benzodioxan-8-yloxy)ethyl]-4-piperidine]-naphthalene-1-sulphonamide

The title compound was prepared starting from amine (IIa-19) and sulphonyl chloride (IIb-17). MS: 469 [M+H⁺]

### Compound 101. N-[[1-[2-(2,3-dihydro-1,4-benzodioxan-5-yloxy)ethyl]-4-piperidine]-methyl]benzenesulphonamide

The title compound was prepared starting from amine (IIa-20) and sulphonyl chloride (IIb-1). MS: 433 [M+H⁺]

### Compound 102. N-[[1-[2-(2,3-Dihydro-1,4-benzodioxan-5-yloxy)ethyl]-4-piperidine]-methyl]-3-methy-benzenesulphonamide

The title compound was prepared starting from amine (IIa-20) and sulphonyl chloride (IIb-8). MS: 447 [M+H⁺]

### Compound 103. N-[[1-[2-(2,3-dihydro-1,4-benzodioxan-5-yloxy)ethyl]-4-piperidine]-methyl]-3-hydroxybenzenesulphonamide

The title compound was prepared starting from amine (IIIa-20) and sulphonyl chloride (IIb-14). MS: 449 [M+H⁺]

### Compound 104. N-[[1-[2-(2,3-Dihydro-1,4-benzodioxan-5-yloxy)ethyl]-4-piperidine]-methyl]naphthalene-1-sulphonamide

The title compound was prepared starting from amine (IIa-20) and sulphonyl chloride (IIb-17). MS: 483 [M+H⁺]

### Zwiazek 105. N-[[1-[2-(2,3-Dihydro-1,4-benzodioxan-5-yloxy)ethyl]-4-piperidine]-methyl]naphthalene-2-sulphonamide

The title compound was prepared starting from amine (IIa-20) and sulphonyl chloride (IIb-18). MS: 483 [M+H⁺]

### Compound 106. N-[[1-[2-(2-Oxoindolin-4-yl)oxyethyl]pyrrolidin-3-yl]methyl]-naphthalene-2-sulphonamide

The title compound was prepared starting from amine (IIa-21) and sulphonyl chloride (IIb-18). MS: 452 [M+H⁺]

### Compound 107. N-[1-[2-[(2,2-Dimethyl-3H-benzofuran-7-yl)oxy]ethyl]pyrrolidin-3-yl]-3-hydroxybenzenesulphonamide

The title compound was prepared starting from amine (IIa-22) and sulphonyl chloride (IIb-14). MS: 433 [M+H⁺]

### Compound 108. N-[1-[2-[(2,2-Dimethyl-3H-benzofuran-7-yl)oxy]ethyl]pyrrolidin-3-yl]naphthalene-2-sulphonamide

The title compound was prepared starting from amine (IIa-22) and sulphonyl chloride (IIb-18). MS: 467 [M+H⁺]

### Compound 109. N-[1-[2-[(2,2-Dimethyl-3H-benzofuran-7-yl)oxy]ethyl]-4-piperidine]-3-hydroxy-benzenesulphonamide

The title compound was prepared starting from amine (IIa-23) and sulphonyl chloride (IIb-14). MS: 447 [M+H⁺]

### Compound 110. N-[1-[2-[(2,2-Dimethyl-3H-benzofuran-7-yl)oxy]ethyl]-4-piperidine]naphthalene-2-sulphonamide

The title compound was prepared starting from amine (IIa-23) and sulphonyl chloride (IIb-18). MS: 481 [M+H⁺]

### Compound 111. N-[[1-[2-[(2,2-Dimethyl-3H-benzofuran-7-yl)oxy]ethyl]azetidin-3-yl]methyl]-3-hydroxy-benzenesulphonamide

The title compound was prepared starting from amine (IIa-24) and sulphonyl chloride (IIb-14). MS: 481 [M+H⁺]

### Compound 112. N-[[1-[2-[(2,2-Dimethyl-3H-benzofuran-7-yl)oxy]ethyl]azetidin-3-yl]methyl]naphthalene-2-sulphonamide

The title compound was prepared starting from amine (IIa-24) and sulphonyl chloride (IIIb-18). MS: 467 [M+H+]

### Compound 113. N-[[1-[2-[(2,2-Dimethyl-3H-benzofuran-7-yl)oxy]ethyl]pyrrolidin-3-yl]methyl]-3-hydroxy-benzenesulphonamide

The title compound was prepared starting from amine (IIa-25) and sulphonyl chloride (IIb-14). MS: 447 [M+H⁺]

### Compound 114. N-[[1-[2-[(2,2-Dimethyl-3H-benzofuran-7-yl)oxy]ethyl]pyrrolidin-3-yl]methyl]naphthalene-2-sulphonamide

The title compound was prepared starting from amine (IIa-25) and sulphonyl chloride (IIb-18). MS: 481 [M+H⁺]

### Compound 115

### 3-Hydroksy-N-[1-[2-(1-naphthyloxy)ethyl]pyrrolidin-3-yl]benzenesulphonamide

The title compound was prepared starting from amine (IIa-26) and sulphonyl chloride (IIb-14). MS: 413 [M+H⁺]

### Compound 116. N-[1-[2-(1-Naphthyloxy)ethyl]pyrrolidin-3-yl]naphthalene-2-sulphonamide

The title compound was prepared starting from amine (IIa-26) and sulphonyl chloride (IIb-18). MS: 447 [M+H⁺]

### Compound 117. N-[1-[2-(2,3-Dihydro-1,4-benzodioxan-8-yloxy)ethyl]-4-piperidine]-benzenesulphonamide

The title compound was prepared starting from amine (IIa-19) and sulphonyl chloride (IIb-1). MS: 418 [M+H⁺]

### Compound 118. N-[1-[2-(2,3-Dihydro-1,4-benzodioxan-8-yloxy)ethyl]-4-piperidine]-3-methyl-benzenesulphonamide

The title compound was prepared starting from amine (IIIa-19) and sulphonyl chloride (IIb-8). MS: 433 [M+H⁺]

### Compound 119. 3-Hydroxy-N-[1-[2-(1-naphthyloxy)ethyl]-4-piperidine]benzene-sulphonamide

The title compound was prepared starting from amine (IIa-27) and sulphonyl chloride (IIb-14). MS: 427 [M+H⁺]

### Compound 120. N-[1-[2-(1-Naphthyloxy)ethyl]-4-piperidine]naphthalene-2-sulphonamide

The title compound was prepared starting from amine (IIa-27) and sulphonyl chloride (IIb-18). MS: 461 [M+H⁺]

### Compound 121. 3-Hydroxy-N-[[1-[2-(1-naphthyloxy)ethyl]azetidin-3-yl]methyl]-benzenesulphonamide

The title compound was prepared starting from amine (IIIa-28) and sulphonyl chloride (IIb-14). MS: 413 [M+H⁺]

### Compound 122. N-[[1-[2-(1-Naphthyloxy)ethyl]azetidin-3-yl]methyl]naphthalene-2-sulphonamide

The title compound was prepared starting from amine (IIa-28) and sulphonyl chloride (IIb-18). MS: 447 [M+H⁺]

### Compound 123. 3-Hydroxy-N-[[1-[2-(1-naphthyloxy)ethyl]pyrrolidin-3-yl]methyl]-benzenesulphonamide

The title compound was prepared starting from amine (IIa-29) and sulphonyl chloride (IIb-14). MS: 427 [M+H⁺]

### Compound 124. N-[[1-[2-(1-Naphthyloxy)ethyl]pyrrolidin-3-yl]methyl]naphthalene-2-sulphonamide

The title compound was prepared starting from amine (IIa-29) and sulphonyl chloride (IIb-18). MS: 461 [M+H⁺]

### Compound 125. N-[1-[2-(1,2-Benzothiazol-3-yloxy)ethyl]pyrrolidin-3-yl]-3-hydroxy-benzenesulphonamide

The title compound was prepared starting from amine (IIa-5) and sulphonyl chloride (IIIb-14). MS: 450 [M+H⁺]

### Compound 126. N-[[1-[3-(6-Fluoro-1,2-benzoxazol-3-yl)propyl]azetidin-3-yl]methyl]-1,3-benzodioxole-5-sulphonamide

The title compound was prepared starting from amine (IIa-30) and sulphonyl chloride (IIIb-48). MS: 420 [M+H⁺]

### Compound 127. N-[[1-[3-(6-Fluoro-1,2-benzoxazol-3-yl)propyl]azetidin-3-yl]methyl]benzo-thiophene-2-sulphonamide

The title compound was prepared starting from amine (IIa-30) and sulphonyl chloride (IIb-22). MS: 460 [M+H⁺]

### Compound 128. N-[[1-[3-(6-Fluoro-1,2-benzoxazol-3-yl)propyl]azetidin-3-yl]methyl]-1,3-benzothiazole-4-sulphonamide

The title compound was prepared starting from amine (IIa-30) and sulphonyl chloride (IIb-28). MS: 461 [M+H⁺]

### Compound 129. 6-chloro-N-[[1-[3-(6-fluoro-1,2-benzoxazol-3-yl)propyl]azetidin-3-yl]methyl]naphthalene-2-sulphonamide

The title compound was prepared starting from amine (IIa-30) and sulphonyl chloride (IIb-37). MS: 488 [M+H⁺]

### Compound 130. 5-Fluoro-N-[[1-[3-(6-fluoro-1,2-benzoxazol-3-yl)propyl]azetidin-3-yl]methyl]-3-methyl-benzothiophene-2-sulphonamide

The title compound was prepared starting from amine (IIIa-30) and sulphonyl chloride (IIb-25). MS: 492 [M+H⁺]

### Compound 131. 5-Chloro-N-[[1-[3-(6-fluoro-1,2-benzoxazol-3-yl)propyl]azetidin-3-yl]-methyl]-3-methyl-benzothiophene-2-sulphonamide

The title compound was prepared starting from amine (IIa-30) and sulphonyl chloride (IIb-26). MS: 508 [M+H⁺]

### Compound 132. N-[[1-[3-(6-Fluoro-1,2-benzoxazol-3-yl)propyl]azetidin-3-yl]methyl]-1,3-benzothiazole-5-sulphonamide

The title compound was prepared starting from amine (IIa-30) and sulphonyl chloride (IIb-55). MS: 461 [M+H⁺]

### Compound 133. N-[[1-[3-(6-Fluoro-1,2-benzoxazol-3-yl)propyl]azetidin-3-yl]methyl]-naphthalene-1-sulphonamide

The title compound was prepared starting from amine (IIa-30) and sulphonyl chloride (IIb-17). MS: 454 [M+H⁺]

### Compound 134. N-[[1-[3-(6-Fluoro-1,2-benzoxazol-3-yl)propyl]azetidin-3-yl]methyl]-naphthalene-2-sulphonamide

The title compound was prepared starting from amine (IIa-30) and sulphonyl chloride (IIb-18). MS: 454 [M+H⁺]

### Compound 135. N-[[1-[3-(6-Fluoro-1,2-benzoxazol-3-yl)propyl]azetidin-3-yl]methyl]-4-phenyl-benzenesulphonamide

The title compound was prepared starting from amine (IIa-30) and sulphonyl chloride (IIb-36). MS: 480 [M+H⁺]

### Compound 136. 4-Chloro-N-[[1-[3-(6-fluoro-1,2-benzoxazol-3-yl)propyl]azetidin-3-yl]methyl]benzenesulphonamide

The title compound was prepared starting from amine (IIa-30) and sulphonyl chloride (IIb-5). MS: 438 [M+H+]

### Compound 137. 3-Chloro-N-[[1-[3-(6-fluoro-1,2-benzoxazol-3-yl)propyl]azetidin-3-yl]methyl]benzenesulphonamide

The title compound was prepared starting from amine (IIa-30) and sulphonyl chloride (IIb-4). MS: 438 [M+H⁺]

### Compound 138. N-[[1-[3-(6-Fluoro-1,2-benzoxazol-3-yl)propyl]azetidin-3-yl]methyl]-4-methyl-benzenesulphonamide

The title compound was prepared starting from amine (IIa-30) and sulphonyl chloride (IIb-33). MS: 418 [M+H⁺]

### Compound 139. N-[[1-[3-(6-Fluoro-1,2-benzoxazol-3-yl)propyl]azetidin-3-yl]methyl]-benzenesulphonamide

The title compound was prepared starting from amine (IIa-30) and sulphonyl chloride (IIb-1). MS: 404 [M+H⁺]

### Compound 140. N-[[1-[3-(6-Fluoro-1,2-benzoxazol-3-yl)propyl]azetidin-3-yl]methyl]-4-(trifluoromethyl)benzenesulphonamide

The title compound was prepared starting from amine (IIa-30) and sulphonyl chloride (IIb-12). MS: 472 [M+H⁺]

### Compound 141. 4-tert-Butyl-N-[[1-[3-(6-fluoro-1,2-benzoxazol-3-yl)propyl]azetidin-3-yl]methyl]benzenesulphonamide

The title compound was prepared starting from amine (IIa-30) and sulphonyl chloride (IIb-10). MS: 460 [M+H⁺]

### Compound 142. N-[[1-[3-(6-Fluoro-1,2-benzoxazol-3-yl)propyl]azetidin-3-yl]methyl]-3-methyl-benzenesulphonamide

The title compound was prepared starting from amine (IIa-30) and sulphonyl chloride (IIb-8). MS: 418 [M+H⁺]

### Compound 143. N-[[1-[3-(6-Fluoro-1,2-benzoxazol-3-yl)propyl]azetidin-3-yl]methyl]-3-methoxy-benzenesulphonamide

The title compound was prepared starting from amine (IIa-30) and sulphonyl chloride (IIb-13). MS: 434 [M+H⁺]

### Compound 144. 3-Fluoro-N-[[1-[3-(6-fluoro-1,2-benzoxazol-3-yl)propyl]azetidin-3-yl]methyl]benzenesulphonamide

The title compound was prepared starting from amine (IIa-30) and sulphonyl chloride (IIb-2). MS: 422 [M+H⁺]

### Compound 145. 4-Cyano-N-[[1-[3-(6-fluoro-1,2-benzoxazol-3-yl)propyl]azetidin-3-yl]methyl]benzenesulphonamide

The title compound was prepared starting from amine (IIIa-30) and sulphonyl chloride (IIb-16). MS: 429 [M+H⁺]

### Compound 146. 3,4-Dichloro-N-[[1-[3-(6-fluoro-1,2-benzoxazol-3-yl)propyl]azetidin-3-yl]methyl]benzenesulphonamide

The title compound was prepared starting from amine (IIa-30) and sulphonyl chloride (IIb-3). MS: 422 [M+H⁺]

### Compound 147. 4-Fluoro-N-[[1-[3-(6-fluoro-1,2-benzoxazol-3-yl)propyl]azetidin-3-yl]methyl]benzenesulphonamide

The title compound was prepared starting from amine (IIIa-30) and sulphonyl chloride (IIb-32). MS: 472 [M+H⁺]

### Compound 148. 4-Bromo-N-[[1-[3-(6-fluoro-1,2-benzoxazol-3-yl)propyl]azetidin-3-yl]methyl]benzenesulphonamide

The title compound was prepared starting from amine (IIa-30) and sulphonyl chloride (IIb-6). MS: 482 [M+H⁺]

### Compound 149. N-[[1-[3-(6-Fluoro-1,2-benzoxazol-3-yl)propyl]azetidin-3-yl]methyl]-3-hydroxy-benzenesulphonamide

The title compound was prepared starting from amine (IIa-30) and sulphonyl chloride (IIb-14). MS: 420 [M+H⁺]

### Compound 150. N-[[1-[3-(6-Fluoro-1,2-benzoxazol-3-yl)propyl]azetidin-3-yl]methyl]-1-methyl-indole-5-sulphonamide

The title compound was prepared starting from amine (IIa-30) and sulphonyl chloride (IIb-51). MS: 457 [M+H⁺]

### Compound 151. N-[[1-[3-(6-Fluoro-1,2-benzoxazol-3-yl)propyl]azetidin-3-yl]methyl]-benzofuran-2-sulphonamide

The title compound was prepared starting from amine (IIa-30) and sulphonyl chloride (IIb-52). MS: 444 [M+H⁺]

### Compound 152. N-[[1-[3-(6-Fluoro-1,2-benzoxazol-3-yl)propyl]azetidin-3-yl]methyl]-1-methyl-indole-4-sulphonamide

The title compound was prepared starting from amine (IIa-30) and sulphonyl chloride (IIb-50). MS: 456 [M+H⁺]

### Compound 153. N-[[1-[3-(6-Fluoro-1,2-benzoxazol-3-yl)propyl]azetidin-3-yl]methyl]-benzothiophene-2-sulphonamide

The title compound was prepared starting from amine (IIa-30) and sulphonyl chloride (IIb-23). MS: 460 [M+H⁺]

### Compound 154. N-[[1-[3-(6-Fluoro-1,2-benzoxazol-3-yl)propyl]azetidin-3-yl]methyl]-thiophene-3-sulphonamide

The title compound was prepared starting from amine (IIa-30) and sulphonyl chloride (IIb-41). MS: 410 [M+H⁺]

### Compound 155. 5-Chloro-N-[[1-[3-(6-fluoro-1,2-benzoxazol-3-yl)propyl]azetidin-3-yl]methyl]thiophene-2-sulphonamide

The title compound was prepared starting from amine (IIa-30) and sulphonyl chloride (IIb-20). MS: 444 [M+H⁺]

### Compound 156. 3-Chloro-4-fluoro-N-[[1-[3-(6-fluoro-1,2-benzoxazol-3-yl)propyl]azetidin-3-yl]methyl]benzenesulphonamide

The title compound was prepared starting from amine (IIa-30) and sulphonyl chloride (IIb-7). MS: 456 [M+H⁺]

### Compound 157. N-[[1-[3-(6-Fluoro-1,2-benzoxazol-3-yl)propyl]azetidin-3-yl]methyl]-4-propyl-benzenesulphonamide

The title compound was prepared starting from amine (IIa-30) and sulphonyl chloride (IIb-9). MS: 446 [M+H⁺]

### Compound 158. 3,4-Difluoro-N-[[1-[3-(6-fluoro-1,2-benzoxazol-3-yl)propyl]azetidin-3-yl]methyl]benzenesulphonamide

The title compound was prepared starting from amine (IIa-30) and sulphonyl chloride (IIb-31). MS: 440 [M+H⁺]

### Compound 159. N-[[1-[3-(6-Fluoro-1,2-benzoxazol-3-yl)propyl]azetidin-3-yl]methyl]-4-(trifluoromethoxy)benzenesulphonamide

The title compound was prepared starting from amine (IIa-30) and sulphonyl chloride (IIIb-35). MS: 488 [M+H⁺]

### Compound 160. N-[[1-[3-(5-Fluoro-1H-indol-3-yl)propyl]azetidin-3-yl]methyl]naphthalene-2-sulphonamide

The title compound was prepared starting from amine (IIa-34) and sulphonyl chloride (IIb-18). MS: 452 [M+H⁺]

### Compound 161. N-[[1-[3-(5-Chloro-1H-indol-3-yl)propyl]azetidin-3-yl]methyl]naphthalene-2-sulphonamide

The title compound was prepared starting from amine (IIa-36) and sulphonyl chloride (IIb-18). MS: 468 [M+H⁺]

### Compound 162. N-[[1-[2-(1,2-Benzothiazol-3-yloxy)ethyl]azetidin-3-yl]methyl]-naphthalene-2-sulphonamide

The title compound was prepared starting from amine (IIa-7) and sulphonyl chloride (IIb-18). MS: 454 [M+H⁺]

### Compound 163. N-[[1-[2-(1-Naphthyloxy)ethyl]azetidin-3-yl]methyl]benzothiophene-2-sulphonamide

The title compound was prepared starting from amine (IIa-28) and sulphonyl chloride (IIb-22). MS: 453 [M+H⁺]

### Compound 164. 6-Chloro-N-[[1-[2-(1-naphthyloxy)ethyl]azetidin-3-yl]methyl]-benzothiophene-2-sulphonamide

The title compound was prepared starting from amine (IIa-28) and sulphonyl chloride (IIb-24). MS: 487 [M+H⁺]

### Compound 165. 6-Chloro-N-[[1-[2-(1-naphthyloxy)ethyl]azetidin-3-yl]methyl]naphthalene-2-sulphonamide

The title compound was prepared starting from amine (IIa-28) and sulphonyl chloride (IIb-37). MS: 481 [M+H⁺]

### Compound 166. 5-Fluoro-3-methyl-N-[[1-[2-(1-naphthyloxy)ethyl]azetidin-3-yl]methyl]benzothiophene-2-sulphonamide

The title compound was prepared starting from amine (IIa-28) and sulphonyl chloride (IIb-25). MS: 485 [M+H⁺]

### Compound 167. 5-Chloro-3-methyl-N-[[1-[2-(1-naphthyloxy)ethyl]azetidin-3-yl]methyl]benzothiophene-2-sulphonamide

The title compound was prepared starting from amine (IIa-28) and sulphonyl chloride (IIb-26). MS: 501 [M+H⁺]

### Compound 168. N-[[1-[2-(1-Naphthyloxy)ethyl]azetidin-3-yl]methyl]naphthalene-1-sulphonamide

The title compound was prepared starting from amine (IIa-28) and sulphonyl chloride (IIb-17). MS: 447 [M+H⁺]

### Compound 169. 1-Methyl-N-[[1-[2-(1-naphthyloxy)ethyl]azetidin-3-yl]methyl]indole-5-sulphonamide

The title compound was prepared starting from amine (IIa-28) and sulphonyl chloride (IIb-51). MS: 450 [M+H⁺]

### Compound 170. 1-Methyl-N-[[1-[2-(1-naphthyloxy)ethyl]azetidin-3-yl]methyl]indole-4-sulphonamide

The title compound was prepared starting from amine (IIa-28) and sulphonyl chloride (IIb-50). MS: 450 [M+H⁺]

### Compound 171. N-[[1-[2-(1-Naphthyloxy)ethyl]azetidin-3-yl]methyl]benzothiophene-3-sulphonamide

The title compound was prepared starting from amine (IIa-28) and sulphonyl chloride (IIb-23). MS: 453 [M+H⁺]

### Compound 172. 3-Chloro-4-fluoro-N-[[1-[2-(1-naphthyloxy)ethyl]azetidin-3-yl]methyl]-benzenesulphonamide

The title compound was prepared starting from amine (IIa-28) and sulphonyl chloride (IIb-7). MS: 449 [M+H⁺]

### Compound 173. 3,4-Difluoro-N-[[1-[2-(1-naphthyloxy)ethyl]azetidin-3-yl]methyl]-benzenesulphonamide

The title compound was prepared starting from amine (IIa-28) and sulphonyl chloride (IIb-31). MS: 433 [M+H⁺]

### Compound 174. 6-Chloro-N-[[1-[2-(2,3-dihydro-1,4-benzodioxin-5-yloxy)ethyl]azetidin-3-yl]methyl]naphthalene-2-sulphonamide

The title compound was prepared starting from amine (IIIa-37) and sulphonyl chloride (IIb-37). MS: 489 [M+H⁺]

### Compound 175. 5-Chloro-N-[[1-[2-(2,3-dihydro-1,4-benzodioxin-5-yloxy)ethyl]azetidin-3-yl]methyl]-3-methyl-benzothiophene-2-sulphonamide

The title compound was prepared starting from amine (IIIa-37) and sulphonyl chloride (IIb-26). MS: 509 [M+H⁺]

### Compound 176. N-[[1-[2-(2,3-Dihydro-1,4-benzodioxin-5-yloxy)ethyl]azetidin-3-yl]methyl]-naphthalene-1-sulphonamide

The title compound was prepared starting from amine (IIa-37) and sulphonyl chloride (IIb-17). MS: 455 [M+H⁺]

### Compound 177. N-[[1-[2-(2,3-Dihydro-1,4-benzodioxin-5-yloxy)ethyl]azetidin-3-yl]methyl]-naphthalene-2-sulphonamide

The title compound was prepared starting from amine (IIIa-37) and sulphonyl chloride (IIb-18). MS: 455 [M+H⁺]

### Compound 178. N-[[1-[2-(2,3-Dihydro-1,4-benzodioxin-5-yloxy)ethyl]azetidin-3-yl]methyl]-4-phenyl-benzenesulphonamide

The title compound was prepared starting from amine (IIa-37) and sulphonyl chloride (IIb-36). MS: 481 [M+H⁺]

### Compound 179. 4-Chloro-N-[[1-[2-(2,3-dihydro-1,4-benzodioxin-5-yloxy)ethyl]azetidin-3-yl]methyl]benzenesulphonamide

The title compound was prepared starting from amine (IIa-37) and sulphonyl chloride (IIb-5). MS: 439 [M+H⁺]

### Compound 180. N-[[1-[2-(2,3-Dihydro-1,4-benzodioxin-5-yloxy)ethyl]azetidin-3-yl]methyl]-4-(trifluoromethyl)benzenesulphonamide

The title compound was prepared starting from amine (IIIa-37) and sulphonyl chloride (IIb-12). MS: 473 [M+H⁺]

### Compound 181. 4-tert-Butyl-N-[[1-[2-(2,3-dihydro-1,4-benzodioxin-5-yloxy)ethyl]azetidin-3-yl]methyl]benzenesulphonamide

The title compound was prepared starting from amine (IIa-37) and sulphonyl chloride (IIb-10). MS: 461 [M+H⁺]

### Compound 182. N-[[1-[2-(2,3-Dihydro-1,4-benzodioxin-5-yloxy)ethyl]azetidin-3-yl]methyl]-4-fluoro-benzenesulphonamide

The title compound was prepared starting from amine (IIIa-37) and sulphonyl chloride (IIb-3). MS: 423 [M+H⁺]

### Compound 183. 3,4-Dichloro-N-[[1-[2-(2,3-dihydro-1,4-benzodioxin-5-yloxy)ethyl]azetidin-3-yl]methyl]benzenesulphonamide

The title compound was prepared starting from amine (IIa-37) and sulphonyl chloride (IIb-32). MS: 473 [M+H⁺]

### Compound 184. N-[[1-[2-(2,3-Dihydro-1,4-benzodioxin-5-yloxy)ethyl]azetidin-3-yl]methyl]-thiophene-2-sulphonamide

The title compound was prepared starting from amine (IIa-37) and sulphonyl chloride (IIb-40). MS: 411 [M+H⁺]

### Compound 185. 4-Bromo-N-[[1-[2-(2,3-dihydro-1,4-benzodioxin-5-yloxy)ethyl]azetidin-3-yl]methyl]benzenesulphonamide

The title compound was prepared starting from amine (IIa-37) and sulphonyl chloride (IIb-6). MS: 483 [M+H⁺]

### Compound 186. N-[[1-[2-(2,3-Dihydro-1,4-benzodioxin-5-yloxy)ethyl]azetidin-3-yl]methyl]-benzofuran-2-sulphonamide

The title compound was prepared starting from amine (IIa-37) and sulphonyl chloride (IIb-52). MS: 445 [M+H⁺]

### Compound 187. N-[[1-[2-(2,3-Dihydro-1,4-benzodioxin-5-yloxy)ethyl]azetidin-3-yl]methyl]-1-methyl-indole-5-sulphonamide

The title compound was prepared starting from amine (IIa-37) and sulphonyl chloride (IIb-51). MS: 458 [M+H⁺]

### Compound 188. N-[[1-[2-(2,3-Dihydro-1,4-benzodioxin-5-yloxy)ethyl]azetidin-3-yl]methyl]-1-methyl-indole-4-sulphonamide

The title compound was prepared starting from amine (IIa-37) and sulphonyl chloride (IIb-50). MS: 458 [M+H⁺]

### Compound 189. N-[[1-[2-(2,3-Dihydro-1,4-benzodioxin-5-yloxy)ethyl]azetidin-3-yl]methyl]-2-oxo-indoline-5-sulphonamide

The title compound was prepared starting from amine (IIIa-37) and sulphonyl chloride (IIb-47). MS: 460 [M+H⁺]

### Compound 190. N-[[1-[2-(2,3-Dihydro-1,4-benzodioxin-5-yloxy)ethyl]azetidin-3-yl]methyl]-benzothiophene-3-sulphonamide

The title compound was prepared starting from amine (IIa-37) and sulphonyl chloride (IIb-23). MS: 461 [M+H⁺]

### Compound 191. N-[[1-[2-(2,3-Dihydro-1,4-benzodioxin-5-yloxy)ethyl]azetidin-3-yl]methyl]-thiophene-3-sulphonamide

The title compound was prepared starting from amine (IIa-37) and sulphonyl chloride (IIb-41). MS: 411 [M+H⁺]

### Compound 192. 5-Chloro-N-[[1-[2-(2,3-dihydro-1,4-benzodioxin-5-yloxy)ethyl]azetidin-3-yl]methyl]thiophene-2-sulphonamide

The title compound was prepared starting from amine (IIa-37) and sulphonyl chloride (IIb-20). MS: 445 [M+H⁺]

### Compound 193. N-[[1-[2-(2,3-Dihydro-1,4-benzodioxin-5-yloxy)ethyl]azetidin-3-yl]methyl]-4-iodo-benzenesulphonamide

The title compound was prepared starting from amine (IIa-37) and sulphonyl chloride (IIb-30). MS: 531 [M+H⁺]

### Compound 194. N-[1-[3-(6-Fluoro-1,2-benzoxazol-3-yl)propyl]pyrrolidin-3-yl]-1,3-benzo-dioxole-5-sulphonamide

The title compound was prepared starting from amine (IIIa-3) and sulphonyl chloride (IIb-48).

¹H-NMR (300 MHz, CDCl₃): δ 7.60-7.56 (m, 1H), 7.40 (dd, 1H, J=1.8 and 8.2 Hz) 7.26-7.20 (m, 1H), 7.20 (dd, 1H, J=1.8 and 8.4 Hz), 7.04 (dt, 1H, J=2.0 and 8.7 Hz), 6.82 (d, 1H, J=8.2 Hz), 6.03 (s, 2H), 3.78 (s, 1H), 2.98 (t, 2H, J=7.4 Hz), 2.79-2.69 (m, 1H), 2.50-2.40 (m, 3H), 2.26-2.18 (m, 1H), 2.10-2.00 (m, 1H), 1.98-1.80 (m, 2H), 1.60-1.49 (m, 2H); MS: 448 [M+H⁺].

### Compound 195. N-[1-[3-(6-Fluoro-1,2-benzoxazol-3-yl)propyl]pyrrolidin-3-yl]-4-phenyl-benzenesulphonamide

The title compound was prepared starting from amine (IIa-3) and sulphonyl chloride (IIb-36). MS: 480 [M+H⁺]

### Compound 196. N-[(3R)-1-[3-(6-Fluoro-1,2-benzoxazol-3-yl)propyl]pyrrolidin-3-yl]benzo-thiophene-2-sulphonamide

The title compound was prepared starting from amine (IIa-32) and sulphonyl chloride (IIb-22).

¹H-NMR (300 MHz, CDCl₃): δ 7.87-7.80 (m, 3H), 7.58-7.54 (m, 1H), 7.48-7.42 (m, 2H), 7.24-7.19 (m, 1H), 7.04 (dt, 1H, J=2.3 and 8.9 Hz), 4.00-3.92 (m, 1H), 2.96 (t, 2H, J=7.4 Hz), 2.84-2.76 (m, 1H), 2.60-2.38 (m, 3H), 2.20-2.08 (m, 2H), 2.00-1.88 (m, 2H), 1.64-1.54 (m, 2H); MS: 460 [M+H⁺].

### Compound 197. N-[(3S)-1-[3-(6-Fluoro-1,2-benzoxazol-3-yl)propyl]pyrrolidin-3-yl]benzo-thiophene-2-sulphonamide

The title compound was prepared starting from amine (IIa-33) and sulphonyl chloride (IIb-22).

¹H-NMR (300 MHz, CDCl₃): δ 7.87-7.80 (m, 3H), 7.58-7.54 (m, 1H), 7.47-7.42 (m, 2H), 7.23-7.18 (m, 1H), 7.04 (dt, 1H, J=1.8 and 8.4 Hz), 4.00-3.92 (m, 1H), 2.95 (t, 2H, J=7.4 Hz), 2.82-2.78 (m, 1H), 2.60-2.54 (m, 1H), 2.50-2.38 (m, 2H), 2.20-2.06 (m, 2H), 2.00-1.88 (m, 2H), 1.64-1.54 (m, 2H); MS: 460 [M+H⁺].

### Compound 198. 6-Chloro-N-[(3R)-1-[3-(6-fluoro-1,2-benzoxazol-3-yl)propyl]pyrrolidin-3-yl]benzothiophene-2-sulphonamide

The title compound was prepared starting from amine (IIIa-32) and sulphonyl chloride (IIb-24).

¹H-NMR (300 MHz, CDCl₃): δ 7.82-7.74 (m, 3H), 7.58-7.54 (m, 1H), 7.39 (dd, 1H, J=1.7 and 8.7 Hz), 7.21 (dd, 1H, J=1.5 and 8.4 Hz), 7.04 (dt, 1H, J=2.0 and 8.9 Hz), 4.00-3.92 (m, 1H), 2.95 (t, 2H, J=8.4 Hz), 2.84-2.78 (m, 1H), 2.58 (dd, 1H, J=2.8 and 9.7 Hz), 2.50-2.38 (m, 2H), 2.20-2.10 (m, 2H), 2.0-1.90 (m, 2H), 1.68-1.58 (m, 2H); MS: 494 [M+H⁺].

### Compound 199. 6-Chloro-N-[(3S)-1-[3-(6-fluoro-1,2-benzoxazol-3-yl)propyl]pyrrolidin-3-yl]benzothiophene-2-sulphonamide

The title compound was prepared starting from amine (IIa-33) and sulphonyl chloride (IIb-24).

¹H-NMR (300 MHz, CDCl₃): δ 7.82-7.74 (m, 3H), 7.59-7.54 (m, 1H), 7.40 (dd, 1H, J=1.7 and 8.7 Hz), 7.22 (dd, 1H, J=1.5 and 8.4 Hz), 7.05 (dt, 1H, J=2.0 and 8.9 Hz), 4.00-3.92 (m, 1H), 2.95 (t, 2H, J=8.4 Hz), 2.84-2.78 (m, 1H), 2.59 (dd, 1H, J=2.8 and 9.7 Hz), 2.50-2.38 (m, 2H), 2.20-2.10 (m, 2H), 2.0-1.90 (m, 2H), 1.68-1.58 (m, 2H); MS: 494 [M+H⁺].

### Compound 200. 6-Chloro-N-[(3R)-1-[3-(6-fluoro-1,2-benzoxazol-3-yl)propyl]pyrrolidin-3-yl]naphthalene-2-sulphonamide

The title compound was prepared starting from amine (IIa-32) and sulphonyl chloride (IIb-37).

¹H-NMR (300 MHz, CDCl₃): δ 8.40 (s, 1H), 7.89-7.84 (m, 4H), 7.56-7.50 (m, 2H), 7.21 (dd, 1H, J=1.8 and 8.4 Hz), 7.04 (dt, 1H, J=1.0 and 8.9 Hz), 3.90-3.82 (m, 1H), 2.94 (t, 2H, J=7.4 Hz), 2.78-2.70 (m, 1H), 2.50-2.40 (m, 3H), 2.38-2.30 (m, 1H), 2.18-1.86 (m, 3H), 1.58-1.48 (m, 2H); MS: 488 [M+H⁺].

### Compound 201. 6-Chloro-N-[(3S)-1-[3-(6-fluoro-1,2-benzoxazol-3-yl)propyl]pyrrolidin-3-yl]naphthalene-2-sulphonamide

The title compound was prepared starting from amine (IIa-33) and sulphonyl chloride (IIb-37).

¹H-NMR (300 MHz, CDCl₃): δ 8.40 (s, 1H), 7.88-7.84 (m, 4H), 7.58-7.52 (m, 2H), 7.21 (dd, 1H, J=1.8 and 8.4 Hz), 7.04 (dt, 1H, J=2.0 and 8.9 Hz), 3.90-3.82 (m, 1H), 2.95 (t, 2H, J=7.4 Hz), 2.81-2.73 (m, 1H), 2.52-2.40 (m, 2H), 2.38-2.30 (m, 1H), 2.18-1.86 (m, 4H), 1.58-1.48 (m, 2H); MS: 488 [M+H⁺].

### Compound 202. 5-Fluoro-N-[(3R)-1-[3-(6-fluoro-1,2-benzoxazol-3-yl)propyl]pyrrolidin-3-yl]-3-methyl-benzothiophene-2-sulphonamide

The title compound was prepared starting from amine (IIa-32) and sulphonyl chloride (IIb-25).

¹H-NMR (300 MHz, CDCl₃): δ 7.78-7.74 (m, 1H), 7.60-7.54 (m, 1H), 7.45 (dd, 1H, J=2.0 and 9.2 Hz), 7.25-7.21 (m, 2H), 7.06 (dt, 1H, J=2.0 and 8.9 Hz), 3.90-3.82 (m, 1H), 2.98 (t, 2H, J=8.4 Hz), 2.86-2.78 (m, 1H), 2.63 (s, 3H), 2.58 (dd, 1H, J=2.8 and 9.7 Hz), 2.47 (t, 2H, J=6.9 Hz), 2.38 (dd, 1H, J=5.8 and 9.7 Hz), 2.18-2.10 (m, 2H), 2.00-1.90 (m, 2H), 1.64-1.58 (m, 1H); MS: 492 [M+H⁺].

### Compound 203. 5-Fluoro-N-[(3S)-1-[3-(6-fluoro-1,2-benzoxazol-3-yl)propyl]pyrrolidin-3-yl]-3-methyl-benzothiophene-2-sulphonamide

The title compound was prepared starting from amine (IIa-33) and sulphonyl chloride (IIb-25).

¹H-NMR (300 MHz, CDCl₃): δ 7.77-7.72 (m, 1H), 7.60-7.54 (m, 1H), 7.44 (dd, 1H, J=2.0 and 9.2 Hz), 7.26-7.21 (m, 2H), 7.06 (dt, 1H, J=2.0 and 8.9 Hz), 3.90-3.82 (m, 1H), 2.98 (t, 2H, J=8.4 Hz), 2.86-2.78 (m, 1H), 2.63 (s, 3H), 2.58 (dd, 1H, J=2.8 and 9.7 Hz), 2.47 (t, 2H, J=6.9 Hz), 2.38 (dd, 1H, J=5.8 and 9.7 Hz), 2.18-2.10 (m, 2H), 2.00-1.90 (m, 2H), 1.64-1.58 (m, 1H); MS: 492 [M+H⁺].

### Compound 204. 5-Chloro-N-[(3R)-1-[3-(6-fluoro-1,2-benzoxazol-3-yl)propyl]pyrrolidin-3-yl]-3-methyl-benzothiophene-2-sulphonamide

The title compound was prepared starting from amine (IIa-32) and sulphonyl chloride (IIb-26).

¹H-NMR (300 MHz, CDCl₃): δ 7.77-7.70 (m, 2H), 7.59-7.52 (m, 1H), 7.43 (dd, 1H, J=2.0 and 8.7 Hz), 7.21 (dd, 1H, J=1.7 and 8.4 Hz), 7.05 (dt, 1H, J=2.0 and 8.9 Hz), 4.00-3.93 (m, 1H), 2.98 (t, 2H, J=8.4 Hz), 2.83-2.78 (m, 1H), 2.62 (s, 3H), 2.60-2.42 (m, 1H), 2.50-2.42 (m, 2H), 2.40-2.36 (m, 1H), 2.20-2.14 (m, 2H), 2.00-1.90 (m, 2H), 1.68-1.58 (m, 1H); MS: 508 [M+H⁺].

### Compound 205. 5-Chloro-N-[(3S)-1-[3-(6-fluoro-1,2-benzoxazol-3-yl)propyl]pyrrolidin-3-yl]-3-methyl-benzothiophene-2-sulphonamide

The title compound was prepared starting from amine (IIa-33) and sulphonyl chloride (IIb-26).

¹H-NMR (300 MHz, CDCl₃): δ 7.78-7.70 (m, 2H), 7.59-7.52 (m, 1H), 7.43 (dd, 1H, J=2.0 and 8.7 Hz), 7.21 (dd, 1H, J=1.7 and 8.4 Hz), 7.05 (dt, 1H, J=2.0 and 8.9 Hz), 4.01-3.93 (m, 1H), 2.97 (t, 2H, J=8.4 Hz), 2.83-2.78 (m, 1H), 2.62 (s, 3H), 2.60-2.42 (m, 1H), 2.51-2.42 (m, 2H), 2.40-2.36 (m, 1H), 2.20-2.14 (m, 2H), 2.00-1.90 (m, 2H), 1.68-1.58 (m, 1H); MS: 508 [M+H⁺].

### Compound 206. N-[(3R)-1-[3-(6-Fluoro-1,2-benzoxazol-3-yl)propyl]pyrrolidin-3-yl]naphthalene-2-sulphonamide

The title compound was prepared starting from amine (IIa-32) and sulphonyl chloride (IIb-18).

¹H-NMR (300 MHz, CDCl₃): δ 8.43 (d, 1H, J=1.8 Hz), 7.98-7.80 (m, 4H), 7.68-7.52 (m, 3H), 7.21 (dd, 1H, J=1.8 and 8.4 Hz), 7.04 (dt, 1H, J=2.3 and 8.9 Hz), 3.83 (m, 1H), 2.84 (t, 2H, J=7.4 Hz), 2.78-2.68 (m, 1H), 2.50-2.32 (m, 3H), 2.18-1.85 (m, 4H), 1.58-1.47 (m, 2H); MS: 454 [M+H⁺].

### Compound 207. N-[(3S)-1-[3-(6-Fluoro-1,2-benzoxazol-3-yl)propyl]pyrrolidin-3-yl]-naphthalene-2-sulphonamide

The title compound was prepared starting from amine (IIa-33) and sulphonyl chloride (IIb-18).

¹H-NMR (300 MHz, CDCl₃): δ 8.43 (d, 1H, J=1.8 Hz), 7.90-7.80 (m, 4H), 7.68-7.52 (m, 3H), 7.21 (dd, 1H, J=1.8 and 8.4 Hz), 7.04 (dt, 1H, J=2.3 and 8.4 Hz), 3.88-3.83 (m, 1H), 2.94 (t, 2H, J=7.4 Hz), 2.78-2.68 (m, 1H), 2.40-2.31 (m, 3H), 2.18-1.84 (m, 4H), 1.58-1.48 (m, 2H); MS: 454 [M+H⁺].

### Compound 208. 4-Chloro-N-[1-[3-(6-fluoro-1,2-benzoxazol-3-yl)propyl]pyrrolidin-3-yl]benzenesulphonamide

The title compound was prepared starting from amine (IIa-2) and sulphonyl chloride (IIb-5). MS: 438 [M+H⁺]

### Compound 209. N-[1-[3-(6-Fluoro-1,2-benzoxazol-3-yl)propyl]pyrrolidin-3-yl]-4-methyl-benzenesulphonamide

The title compound was prepared starting from amine (IIa-2) and sulphonyl chloride (IIb-33). MS: 418 [M+H⁺]

### Compound 210. 4-Cyano-N-[1-[3-(6-fluoro-1,2-benzoxazol-3-yl)propyl]pyrrolidin-3-yl]benzenesulphonamide

The title compound was prepared starting from amine (IIa-2) and sulphonyl chloride (IIb-16).

¹H-NMR (300 MHz, CDCl₃): δ 8.01-7.96 (m, 2H), 7.82-7.78 (m, 2H), 7.60-7.54 (m, 1H), 7.24 (dd, 1H, J=2.0 and 8.4 Hz), 7.06 (dt, 1H, J=2.3 and 8.9 Hz), 3.80 (m, 1H), 2.98 (t, 2H, J=7.4 Hz), 2.84-2.78 (m, 1H), 2.52-2.44 (m, 3H), 2.40-2.34 (m, 1H), 2.18-2.02 (m, 2H), 2.00-1.98 (m, 2H), 1.58-1.48 (m, 1H); MS: 429 [M+H⁺].

### Compound 211. 3,4-Dichloro-N-[1-[3-(6-fluoro-1,2-benzoxazol-3-yl)propyl]pyrrolidin-3-yl]benzenesulphonamide

The title compound was prepared starting from amine (IIa-2) and sulphonyl chloride (IIb-32). MS: 429 [M+H⁺]

### Compound 212. N-[1-[3-(6-Fluoro-1,2-benzoxazol-3-yl)propyl]pyrrolidin-3-yl]thiophene-2-sulphonamide

The title compound was prepared starting from amine (IIa-2) and sulphonyl chloride (IIb-40). MS: 410 [M+H⁺]

### Compound 213. N-[1-[3-(6-Fluoro-1,2-benzoxazol-3-yl)propyl]pyrrolidin-3-yl]-4-methoxybenzenesulphonamide

The title compound was prepared starting from amine (IIa-2) and sulphonyl chloride (IIb-34). MS: 434 [M+H⁺]

### Compound 214. N-[(3R)-1-[3-(6-Fluoro-1,2-benzoxazol-3-yl)propyl]pyrrolidin-3-yl]benzo-furan-2-sulphonamide

The title compound was prepared starting from amine (IIa-32) and sulphonyl chloride (IIb-52).

¹H-NMR (300 MHz, CDCl₃): δ 7.67-7.63 (m, 1H), 7.58-7.53 (m, 1H), 7.51-7.48 (m, 1H), 7.45-7.38 (m, 1H), 7.37-7.28 (m, 1H), 7.34-7.28 (m, 1H), 7.20 (dd, 1H, J=1.7 and 8.4 Hz), 7.04 (dt, 1H, J=2.0 and 8.9 Hz), 4.04-3.96 (m, 1H), 2.96 (t, 2H, J=8.4 Hz), 2.84-2.78 (m, 1H), 2.60-2.40 (m, 3H), 2.20-2.08 (m, 2H), 2.00-1.90 (m, 2H), 1.64-1.58 (m, 2H); MS: 444 [M+H⁺].

### Compound 215. N-[(3S)-1-[3-(6-Fluoro-1,2-benzoxazol-3-yl)propyl]pyrrolidin-3-yl]benzo-furan-2-sulphonamide

The title compound was prepared starting from amine (IIa-33) and sulphonyl chloride (IIb-52).

¹H-NMR (300 MHz, CDCl₃): δ 7.67-7.63 (m, 1H), 7.58-7.53 (m, 1H), 7.51-7.48 (m, 1H), 7.45-7.38 (m, 1H), 7.37-7.28 (m, 1H), 7.34-7.28 (m, 1H), 7.20 (dd, 1H, J=1.7 and 8.4 Hz), 7.04 (dt, 1H, J=2.0 and 8.9 Hz), 4.04-3.96 (m, 1H), 2.96 (t, 2H, J=8.4 Hz), 2.84-2.78 (m, 1H), 2.61-2.40 (m, 3H), 2.20-2.08 (m, 2H), 2.00-1.90 (m, 2H), 1.64-1.58 (m, 2H); MS: 444 [M+H⁺].

### Compound 216. N-[1-[3-(6-Fluoro-1,2-benzoxazol-3-yl)propyl]pyrrolidin-3-yl]benzofuran-2-sulphonamide

The title compound was prepared starting from amine (IIa-2) and sulphonyl chloride (IIb-52).

¹H-NMR (300 MHz, CDCl₃): δ 7.68-7.64 (m, 1H), 7.58-7.56 (m, 1H), 7.52-7.48 (m, 1H), 7.46-7.40 (m, 1H), 7.38-7.29 (m, 2H), 7.24-7.20 (m, 1H), 7.05 (dt, 1H, J=2.3 and 8.9 Hz), 4.00 (m, 1H), 2.98 (t, 2H, J=7.4 Hz), 2.84-2.78 (m, 1H), 2.60-2.38 (m, 4H), 2.20-2.08 (m, 2H), 2.00-1.90 (m, 2H), 1.64-1.58 (m, 1H); MS: 444 [M+H⁺].

### Compound 217. N-[1-[3-(6-Fluoro-1,2-benzoxazol-3-yl)propyl]pyrrolidin-3-yl]-1-methyl-imidazole-4-sulphonamide

The title compound was prepared starting from amine (IIa-2) and sulphonyl chloride (IIb-44).

¹H-NMR (300 MHz, CDCl₃): δ 7.70 (dd, 1H, J=1 and 7.70 Hz), 7.54-7.50 (m, 1H), 7.15 (d, 1H, J=3.0 Hz), 7.06 (dd, 1H, J=2.0 and 8.7 Hz), 6.87 (dd, 1H, J=0.7 and 3.0 Hz), 3.80 (s, 3H), 3.78 (m, 1H), 2.98 (t, 2H, J=7.4 Hz), 2.72-2.62 (m, 1H), 2.46-2.30 (m, 3H), 2.12-2.02 (m, 2H), 1.98-1.78 (m, 3H), 1.48-1.38 (m, 1H); MS: 458 [M+H⁺].

### Compound 218. N-[1-[3-(6-Fluoro-1,2-benzoxazol-3-yl)propyl]pyrrolidin-3-yl]-1-methyl-indole-5-sulphonamide

The title compound was prepared starting from amine (IIa-2) and sulphonyl chloride (IIb-51).

¹H-NMR (300 MHz, CDCl₃): δ 8.18 (d, 1H, J=1.3 Hz), 7.66 (dd, 1H, J=1.8 and 8.7 Hz), 7.59-7.54 (m, 1H), 7.38-7.34 (m, 1H), 7.23-7.14 (m, 2H), 7.04 (dt, 1H, J=2.0 and 8.7 Hz), 6.56 (dt, 1H, J=0.7 and 3.0 Hz), 3.80 (m, 4H), 2.94 (t, 2H, J=7.4 Hz), 2.78-2.64 (m, 1H), 2.50-2.34 (m, 4H), 2.22-2.18 (m, 1H), 2.08-1.88 (m, 3H), 1.58-1.48 (m, 1H); MS: 458 [M+H⁺].

### Compound 219. N-[1-[3-(6-Fluoro-1,2-benzoxazol-3-yl)propyl]pyrrolidin-3-yl]-1-methyl-indole-4-sulphonamide

The title compound was prepared starting from amine (IIIa-2) and sulphonyl chloride (IIb-50). MS: 457 [M+H⁺]

### Compound 220. N-[1-[3-(6-Fluoro-1,2-benzoxazol-3-yl)propyl]pyrrolidin-3-yl]-2-oxo-indoline-5-sulphonamide

The title compound was prepared starting from amine (IIa-2) and sulphonyl chloride (IIb-47).

¹H-NMR (300 MHz, DMSO): δ 10.78 (s, 1H), 7.98-7.80 (m, 1H), 7.64-7.58 (m, 3H), 7.24-7.18 (m, 1H), 6.96-6.90 (dt, 1H, J=2.3 and 8.9 Hz), 3.60 (s, 2H), 3.56 (s, 1H), 2.94 (t, 2H, J=7.4 Hz), 2.56 (m, 1H), 2.46-2.30 (m, 4H), 2.24-2.18 (m, 1H), 1.88-1.74 (m, 2H), 1.47-1.38 (m, 2H); MS: 459 [M+H⁺].

### Compound 221. N-[1-[3-(6-Fluoro-1,2-benzoxazol-3-yl)propyl]pyrrolidin-3-yl]-2,5-dimethyl-thiophene-3-sulphonamide

The title compound was prepared starting from amine (IIa-2) and sulphonyl chloride (IIb-42).

¹H-NMR (300 MHz, CDCl₃): δ 7.62-7.58 (m, 1H), 7.28-7.21 (m, 1H), 7.06 (dt, 1H, J=2.3 and 8.9 Hz), 6.87 (d, 1H, J=1.0 Hz), 3.81-3.78 (m, 1H), 2.98 (t, 2H, J=7.4 Hz), 2.80-2.72 (m, 1H), 2.59 (s, 3H), 2.54-2.46 (m, 3H), 2.38 (s, 3H), 2.21-2.06 (m, 2H), 2.02-1.90 (m, 2H), 1.62-1.58 (m, 2H); MS: 438 [M+H⁺].

### Compound 222. N-[(3R)-1-[3-(6-Fluoro-1,2-benzoxazol-3-yl)propyl]pyrrolidin-3-yl]-2,5-dimethyl-thiophene-3-sulphonamide

The title compound was prepared starting from amine (IIa-32) and sulphonyl chloride (IIb-42).

¹H-NMR (300 MHz, CDCl₃): δ 7.62-7.58 (m, 1H), 7.23-7.19 (m, 1H), 7.05 (dt, 1H, J=2.3 and 8.9 Hz), 6.87-6.85 (m, 1H), 3.81-3.78 (m, 1H), 2.98 (t, 2H, J=8.4 Hz), 2.80-2.72 (m, 1H), 2.58 (s, 3H), 2.54-2.40 (m, 2H), 2.35 (s, 3H), 2.28-2.08 (m, 1H), 2.14-2.04 (m, 2H), 2.02-1.90 (m, 2H), 1.64-1.56 (m, 2H); MS: 438 [M+H⁺].

### Compound 223. N-[(3S)-1-[3-(6-Fluoro-1,2-benzoxazol-3-yl)propyl]pyrrolidin-3-yl]-2,5-dimethyl-thiophene-3-sulphonamide

The title compound was prepared starting from amine (IIIa-33) and sulphonyl chloride (IIb-42).

¹H-NMR (300 MHz, CDCl₃): δ 7.62-7.58 (m, 1H), 7.23-7.19 (m, 1H), 7.05 (dt, 1H, J=2.3 and 8.9 Hz), 6.87-6.85 (m, 1H), 3.81-3.78 (m, 1H), 2.98 (t, 2H, J=8.4 Hz), 2.80-2.72 (m, 1H), 2.58 (s, 3H), 2.54-2.40 (m, 2H), 2.35 (s, 3H), 2.28-2.08 (m, 1H), 2.13-2.04 (m, 2H), 2.00-1.98 (m, 2H), 1.64-1.56 (m, 2H); MS: 438 [M+H⁺].

### Compound 224. N-[(3R)-1-[3-(6-Fluoro-1,2-benzoxazol-3-yl)propyl]pyrrolidin-3-yl]benzothiophene-3-sulphonamide

The title compound was prepared starting from amine (IIa-32) and sulphonyl chloride (IIb-23).

¹H-NMR (300 MHz, CDCl₃): δ 8.23 (s, 1H), 8.18-8.16 (m, 1H), 7.87-7.83 (m, 1H), 7.58-7.52 (m, 1H), 7.48-7.36 (m, 2H), 7.22 (dd, 1H, J=1.8 and 8.4 Hz), 7.04 (dt, 1H, J=2.3 and 8.9 Hz), 3.90-3.80 (m, 1H), 2.80 (t, 2H, J=8.4 Hz), 2.72-2.64 (m, 1H), 2.48-2.24 (m, 3H), 2.18-1.94 (m, 2H), 1.88-1.78 (m, 2H), 1.54-1.40 (m, 2H); MS: 460 [M+H⁺].

### Compound 225. N-[(3S)-1-[3-(6-Fluoro-1,2-benzoxazol-3-yl)propyl]pyrrolidin-3-yl]benzothiophene-3-sulphonamide

The title compound was prepared starting from amine (IIa-33) and sulphonyl chloride (IIb-23).

¹H-NMR (300 MHz, CDCl₃): δ 8.23 (s, 1H), 8.18-8.16 (m, 1H), 7.88-7.84 (m, 1H), 7.58-7.52 (m, 1H), 7.50-7.36 (m, 2H), 7.22 (dd, 1H, J=1.8 and 8.4 Hz), 7.06 (dt, 1H, J=2.3 and 8.9 Hz), 3.90-3.80 (m, 1H), 2.80 (t, 2H, J=8.4 Hz), 2.72-2.64 (m, 1H), 2.5-2.24 (m, 3H), 2.18-1.94 (m, 2H), 1.88-1.78 (m, 2H), 1.54-1.40 (m, 2H); MS: 460 [M+H⁺].

### Compound 226. N-[1-[3-(6-Fluoro-1,2-benzoxazol-3-yl)propyl]pyrrolidin-3-yl]-5-methylbenzothiophene-2-sulphonamide

The title compound was prepared starting from amine (IIa-2) and sulphonyl chloride (IIb-54).

¹H-NMR (300 MHz, CDCl₃): δ 7.77 (d, 1H, J=0.7 Hz), 7.69 (d, 1H, J=8.4 Hz), 7.64-7.62 (m, 1H), 7.59-7.54 (m, 1H), 7.32-7.28 (m, 1H), 7.21 (dd, 1H, J=1.8 and 8.4 Hz), 7.04 (dt, 1H, J=2.0 and 8.9 Hz), 4.00-3.92 (m, 1H), 2.98 (t, 2H, J=7.4 Hz), 2.84-2.76 (m, 1H), 2.60-2.38 (m, 3H), 2.45 (s, 3H), 2.20-2.10 (m, 2H), 1.98-1.78 (m, 2H), 1.66-1.58 (m, 2H); MS: 474 [M+H⁺].

### Compound 227. N-[1-[3-(6-Fluoro-1,2-benzoxazol-3-yl)propyl]pyrrolidin-3-yl]-6-methoxy-naphthalene-2-sulphonamide

The title compound was prepared starting from amine (IIa-2) and sulphonyl chloride (IIb-39).

¹H-NMR (300 MHz, CDCl₃): δ 8.32 (s, 1H), 7.84-7.78 (m, 2H), 7.58-7.52 (m, 1H), 7.24-7.14 (m, 4H), 7.03 (dt, 1H, J=2.0 and 8.9 Hz), 3.95 (s, 3H), 3.90-3.80 (m, 1H), 2.96 (t, 2H, J=8.4 Hz), 2.75-2.68 (m, 1H), 2.49-2.32 (m, 3H), 2.20-1.84 (m, 4H), 1.60-1.48 (m, 2H); MS: 484 [M+H⁺].

### Compound 228. N-[(3R)-1-[3-(6-Fluoro-1,2-benzoxazol-3-yl)propyl]pyrrolidin-3-yl]-5-methyl-benzothiophene-2-sulphonamide

The title compound was prepared starting from amine (IIa-32) and sulphonyl chloride (IIb-54).

¹H-NMR (300 MHz, CDCl₃): δ 7.78 (d, 1H, J=0.7 Hz), 7.70 (d, 1H, J=8.4 Hz), 7.65-7.63 (m, 1H), 7.59-7.54 (m, 1H), 7.32-7.28 (m, 1H), 7.21 (dd, 1H, J=1.8 and 8.4 Hz), 7.04 (dt, 1H, J=2.0 and 8.9 Hz), 3.90-3.82 (m, 1H), 2.98 (t, 2H, J=7.4 Hz), 2.82-2.75 (m, 1H), 2.48-2.38 (m, 3H), 2.45 (s, 3H), 2.18-2.10 (m, 2H), 1.98-1.78 (m, 2H), 1.64-1.58 (m, 2H); MS: 474 [M+H⁺].

### Compound 229. N-[(3S)-1-[3-(6-Fluoro-1,2-benzoxazol-3-yl)propyl]pyrrolidin-3-yl]-5-methylbenzothiophene-2-sulphonamide

The title compound was prepared starting from amine (IIa-33) and sulphonyl chloride (IIb-54).

¹H-NMR (300 MHz, CDCl₃): δ 7.68 (d, 1H, J=0.7 Hz), 7.69 (d, 1H, J=8.4 Hz), 7.64-7.62 (m, 1H), 7.58-7.54 (m, 1H), 7.31-7.28 (m, 1H), 7.21 (dd, 1H, J=1.8 and 8.4 Hz), 7.04 (dt, 1H, J=2.0 and 8.9 Hz), 3.90-3.82 (m, 1H), 2.98 (t, 2H, J=7.4 Hz), 2.82-2.74 (m, 1H), 2.50-2.38 (m, 3H), 2.45 (s, 3H), 2.18-2.10 (m, 2H), 1.98-1.78 (m, 2H), 1.64-1.58 (m, 2H); MS: 474 [M+H⁺].

### Compound 230. N-[(3R)-1-[3-(6-Fluoro-1,2-benzoxazol-3-yl)propyl]pyrrolidin-3-yl]-6-methoxy-naphthalene-2-sulphonamide

The title compound was prepared starting from amine (IIa-32) and sulphonyl chloride (IIb-39).

¹H-NMR (300 MHz, CDCl₃): δ 8.33 (s, 1H), 7.82-7.78 (m, 2H), 7.56-7.50 (m, 1H), 7.22-7.12 (m, 4H), 7.02 (dt, 1H, J=2.0 and 8.9 Hz), 3.95 (s, 3H), 3.90-3.80 (m, 1H), 2.96 (t, 2H, J=8.4 Hz), 2.74-2.68 (m, 1H), 2.49-2.32 (m, 3H), 2.20-2.10 (m, 1H), 2.08-1.98 (m, 1H), 1.94-1.84 (m, 2H), 1.60-1.48 (m, 2H); MS: 484 [M+H⁺].

### Compound 231. N-[(3S)-1-[3-(6-Fluoro-1,2-benzoxazol-3-yl)propyl]pyrrolidin-3-yl]-6-methoxy-naphthalene-2-sulphonamide

The title compound was prepared starting from amine (IIa-33) and sulphonyl chloride (IIb-39).

¹H-NMR (300 MHz, CDCl₃): δ 8.33 (s, 1H), 7.82-7.78 (m, 2H), 7.57-7.51 (m, 1H), 7.23-7.13 (m, 4H), 7.02 (dt, 1H, J=2.0 and 8.9 Hz), 3.95 (s, 3H), 3.90-3.80 (m, 1H), 2.95 (t, 2H, J=8.4 Hz), 2.74-2.68 (m, 1H), 2.49-2.32 (m, 3H), 2.20-2.10 (m, 1H), 2.08-1.98 (m, 1H), 1.94-1.84 (m, 2H), 1.60-1.48 (m, 2H); MS: 484 [M+H⁺].

### Compound 232. 7-Chloro-N-[(3R)-1-[3-(6-fluoro-1,2-benzoxazol-3-yl)propyl]pyrrolidin-3-yl]naphthalene-2-sulphonamide

The title compound was prepared starting from amine (IIa-32) and sulphonyl chloride (IIb-38).

¹H-NMR (300 MHz, CDCl₃): δ 8.40 (s, 1H), 7.89-7.83 (m, 4H), 7.58-7.50 (m, 2H), 7.21 (dd, 1H, J=1.8 and 8.2 Hz), 7.03 (dt, 1H, J=2.0 and 8.9 Hz), 3.92-3.82 (m, 1H), 2.98 (t, 2H, J=8.4 Hz), 2.78-2.70 (m, 1H), 2.50-2.32 (m, 3H), 2.20-1.84 (m, 4H), 1.60-1.58 (m, 2H); MS: 488 [M+H⁺].

### Compound 233. 7-Chloro-N-[(3S)-1-[3-(6-fluoro-1,2-benzoxazol-3-yl)propyl]pyrrolidin-3-yl]naphthalene-2-sulphonamide

The title compound was prepared starting from amine (IIa-33) and sulphonyl chloride (IIb-38).

¹H-NMR (300 MHz, CDCl₃): δ 8.40 (s, 1H), 7.89-7.84 (m, 4H), 7.58-7.50 (m, 2H), 7.21 (dd, 1H, J=1.8 and 8.2 Hz), 7.04 (dt, 1H, J=2.0 and 8.9 Hz), 3.91-3.82 (m, 1H), 2.98 (t, 2H, J=8.4 Hz), 2.80-2.70 (m, 1H), 2.52-2.32 (m, 3H), 2.20-1.84 (m, 4H), 1.60-1.58 (m, 2H); MS: 488 [M+H⁺].

### Compound 234. 6-Fluoro-N-[1-[3-(6-fluoro-1,2-benzoxazol-3-yl)propyl]pyrrolidin-3-yl]benzothiophene-2-sulphonamide

The title compound was prepared starting from amine (IIa-2) and sulphonyl chloride (IIb-53).

¹H-NMR (300 MHz, CDCl₃): δ 7.84-7.79 (m, 2H), 7.60-7.54 (m, 1H), 7.50 (dd, 1H, J=2.3 and 8.4 Hz), 7.24-7.16 (m, 2H), 7.05 (dt, 1H, J=2.0 and 8.7 Hz), 3.98 (m, 1H), 2.98 (t, 2H, J=7.4 Hz), 2.86-2.80 (m, 1H), 2.62-2.58 (m, 2H), 2.52-2.46 (m, 1H), 2.44-2.38 (m, 1H), 2.20-2.10 (m, 2H), 2.00-1.90 (m, 2H), 1.68-1.58 (m, 1H); MS: 478 [M+H⁺].

### Compound 235. N-[[1-[3-(6-Fluoro-1,2-benzoxazol-3-yl)propyl]pyrrolidin-3-yl]methyl]-1,3-benzodioxole-5-sulphonamide

The title compound was prepared starting from amine (IIa-31) and sulphonyl chloride (IIb-48). MS: 462 [M+H⁺]

### Compound 236. N-[[1-[3-(6-Fluoro-1,2-benzoxazol-3-yl)propyl]pyrrolidin-3-yl]methyl]-1,3-benzothiazole-4-sulphonamide

The title compound was prepared starting from amine (IIa-31) and sulphonyl chloride (IIb-28). MS: 475 [M+H⁺]

### Compound 237. 6-Chloro-N-[[1-[3-(6-fluoro-1,2-benzoxazol-3-yl)propyl]pyrrolidin-3-yl]methyl]naphthalene-2-sulphonamide

The title compound was prepared starting from amine (IIIa-31) and sulphonyl chloride (IIb-37). MS: 502 [M+H⁺]

### Compound 238. 5-Chloro-N-[[1-[3-(6-fluoro-1,2-benzoxazol-3-yl)propyl]pyrrolidin-3-yl]methyl]-3-methyl-benzothiophene-2-sulphonamide

The title compound was prepared starting from amine (IIa-31) and sulphonyl chloride (IIb-26). MS: 522 [M+H⁺]

### Compound 239. N-[[1-[3-(6-Fluoro-1,2-benzoxazol-3-yl)propyl]pyrrolidin-3-yl]methyl]naphthalene-1-sulphonamide

The title compound was prepared starting from amine (IIa-31) and sulphonyl chloride (IIb-17). MS: 468 [M+H⁺]

### Compound 240. N-[[1-[3-(6-Fluoro-1,2-benzoxazol-3-yl)propyl]pyrrolidin-3-yl]methyl]-naphthalene-2-sulphonamide

The title compound was prepared starting from amine (IIa-31) and sulphonyl chloride (IIb-18). MS: 468 [M+H⁺]

### Compound 241. N-[[1-[3-(6-Fluoro-1,2-benzoxazol-3-yl)propyl]pyrrolidin-3-yl]methyl]-4-phenyl-benzenesulphonamide

The title compound was prepared starting from amine (IIa-31) and sulphonyl chloride (IIb-36). MS: 494 [M+H⁺]

### Compound 242. 4-Chloro-N-[[1-[3-(6-fluoro-1,2-benzoxazol-3-yl)propyl]pyrrolidin-3-yl]methyl]benzenesulphonamide

The title compound was prepared starting from amine (IIa-31) and sulphonyl chloride (IIb-5). MS: 452 [M+H⁺]

### Compound 243. 3-Chloro-N-[[1-[3-(6-fluoro-1,2-benzoxazol-3-yl)propyl]pyrrolidin-3-yl]methyl]benzenesulphonamide

The title compound was prepared starting from amine (IIIa-31) and sulphonyl chloride (IIb-4). MS: 452 [M+H⁺]

### Compound 244. N-[[1-[3-(6-Fluoro-1,2-benzoxazol-3-yl)propyl]pyrrolidin-3-yl]methyl]-4-methyl-benzenesulphonamide

The title compound was prepared starting from amine (IIa-31) and sulphonyl chloride (IIb-33). MS: 432 [M+H⁺]

### Compound 245. N-[[1-[3-(6-Fluoro-1,2-benzoxazol-3-yl)propyl]pyrrolidin-3-yl]methyl]benzenesulphonamide

The title compound was prepared starting from amine (IIa-31) and sulphonyl chloride (IIb-1). MS: 418 [M+H⁺]

### Compound 246. N-[[1-[3-(6-Fluoro-1,2-benzoxazol-3-yl)propyl]pyrrolidin-3-yl]methyl]-4-(trifluoromethyl)benzenesulphonamide

The title compound was prepared starting from amine (IIa-31) and sulphonyl chloride (IIb-12). MS: 486 [M+H⁺]

### Compound 247. N-[[1-[3-(6-Fluoro-1,2-benzoxazol-3-yl)propyl]pyrrolidin-3-yl]methyl]-3-(trifluoromethyl)benzenesulphonamide

The title compound was prepared starting from amine (IIa-31) and sulphonyl chloride (IIb-11). MS: 486 [M+H⁺]

### Compound 248. 4-tert-Butyl-N-[[1-[3-(6-fluoro-1,2-benzoxazol-3-yl)propyl]pyrrolidin-3-yl]methyl]benzenesulphonamide

The title compound was prepared starting from amine (IIa-31) and sulphonyl chloride (IIb-10). MS: 474 [M+H⁺]

### Compound 249. 3-tert-Butyl-N-[[1-[3-(6-fluoro-1,2-benzoxazol-3-yl)propyl]pyrrolidin-3-yl]methyl]benzenesulphonamide

The title compound was prepared starting from amine (IIa-31) and sulphonyl chloride (IIb-8). MS: 432 [M+H⁺]

### Compound 250. N-[[1-[3-(6-Fluoro-1,2-benzoxazol-3-yl)propyl]pyrrolidin-3-yl]methyl]-3-methoxy-benzenesulphonamide

The title compound was prepared starting from amine (IIa-31) and sulphonyl chloride (IIb-13). MS: 448 [M+H⁺]

### Compound 251. 4-Cyano-N-[[1-[3-(6-fluoro-1,2-benzoxazol-3-yl)propyl]pyrrolidin-3-yl]methyl]benzenesulphonamide

The title compound was prepared starting from amine (IIa-31) and sulphonyl chloride (IIb-16). MS: 443 [M+H⁺]

### Compound 252. 4-Fluoro-N-[[1-[3-(6-fluoro-1,2-benzoxazol-3-yl)propyl]pyrrolidin-3-yl]methyl]benzenesulphonamide

The title compound was prepared starting from amine (IIa-31) and sulphonyl chloride (IIb-3). MS: 436 [M+H⁺]

### Compound 253. 3,4-Dichloro-N-[[1-[3-(6-fluoro-1,2-benzoxazol-3-yl)propyl]pyrrolidin-3-yl]methyl]benzenesulphonamide

The title compound was prepared starting from amine (IIa-31) and sulphonyl chloride (IIb-32). MS: 486 [M+H⁺]

### Compound 254. N-[[1-[3-(6-Fluoro-1,2-benzoxazol-3-yl)propyl]pyrrolidin-3-yl]methyl]-3-hydroxy-benzenesulphonamide

The title compound was prepared starting from amine (IIa-31) and sulphonyl chloride (IIb-14). MS: 434 [M+H⁺]

### Compound 255. N-[[1-[3-(6-Fluoro-1,2-benzoxazol-3-yl)propyl]pyrrolidin-3-yl]methyl]-4-methoxy-benzenesulphonamide

The title compound was prepared starting from amine (IIa-31) and sulphonyl chloride (IIb-34). MS: 448 [M+H⁺]

### Compound 256. N-[[1-[3-(6-Fluoro-1,2-benzoxazol-3-yl)propyl]pyrrolidin-3-yl]methyl]-2,3-dihydrobenzofuran-5-sulphonamide

The title compound was prepared starting from amine (IIa-31) and sulphonyl chloride (IIb-21). MS: 460 [M+H⁺]

### Compound 257. N-[[1-[3-(6-Fluoro-1,2-benzoxazol-3-yl)propyl]pyrrolidin-3-yl]methyl]-benzofuran-2-sulphonamide

The title compound was prepared starting from amine (IIa-31) and sulphonyl chloride (IIb-52). MS: 458 [M+H⁺]

### Compound 258. N-[[1-[3-(6-Fluoro-1,2-benzoxazol-3-yl)propyl]pyrrolidin-3-yl]methyl]-1-methyl-indole-5-sulphonamide

The title compound was prepared starting from amine (IIa-31) and sulphonyl chloride (IIb-51). MS: 473 [M+H⁺]

### Compound 259. N-[[1-[3-(6-Fluoro-1,2-benzoxazol-3-yl)propyl]pyrrolidin-3-yl]methyl]-1-methyl-indole-4-sulphonamide

The title compound was prepared starting from amine (IIa-31) and sulphonyl chloride (IIb-50). MS: 471 [M+H⁺]

### Compound 260. N-[[1-[3-(6-Fluoro-1,2-benzoxazol-3-yl)propyl]pyrrolidin-3-yl]methyl]-2-oxo-indoline-5-sulphonamide

The title compound was prepared starting from amine (IIa-31) and sulphonyl chloride (IIb-47). MS: 473 [M+H⁺]

### Compound 261. N-[[1-[3-(6-Fluoro-1,2-benzoxazol-3-yl)propyl]pyrrolidin-3-yl]methyl]-benzothiophene-3-sulphonamide

The title compound was prepared starting from amine (IIa-31) and sulphonyl chloride (IIb-23). MS: 474 [M+H⁺]

### Compound 262. N-[[1-[3-(6-Fluoro-1,2-benzoxazol-3-yl)propyl]pyrrolidin-3-yl]methyl]-2,5-dimethyl-thiophene-3-sulphonamide

The title compound was prepared starting from amine (IIIa-31) and sulphonyl chloride (IIb-42). MS: 452 [M+H⁺]

### Compound 263. 3-Chloro-4-fluoro-N-[[1-[3-(6-fluoro-1,2-benzoxazol-3-yl)propyl]pyrrolidin-3-yl]methyl]benzenesulphonamide

The title compound was prepared starting from amine (IIIa-31) and sulphonyl chloride (IIb-7). MS: 470 [M+H⁺]

### Compound 264. N-[[1-[3-(6-Fluoro-1,2-benzoxazol-3-yl)propyl]pyrrolidin-3-yl]methyl]-4-propyl-benzenesulphonamide

The title compound was prepared starting from amine (IIa-31) and sulphonyl chloride (IIb-9). MS: 460 [M+H⁺]

### Compound 265. 3,4-Difluoro-N-[[1-[3-(6-fluoro-1,2-benzoxazol-3-yl)propyl]pyrrolidin-3-yl]methyl]benzenesulphonamide

The title compound was prepared starting from amine (IIa-31) and sulphonyl chloride (IIb-31). MS: 454 [M+H⁺]

### Compound 266. N-[[1-[3-(6-Fluoro-1,2-benzoxazol-3-yl)propyl]pyrrolidin-3-yl]methyl]-4-(trifluoromethoxy)benzenesulphonamide

The title compound was prepared starting from amine (IIa-31) and sulphonyl chloride (IIb-35). MS: 502 [M+H⁺]

### Compound 267. N-[[1-[3-(5-Fluoro-1H-indol-3-yl)propyl]pyrrolidin-3-yl]methyl]-naphthalene-2-sulphonamide

The title compound was prepared starting from amine (IIa-35) and sulphonyl chloride (IIb-18). MS: 466 [M+H⁺]

### Compound 268. N-[[1-[2-(1-Naphthyloxy)ethyl]pyrrolidin-3-yl]methyl]benzothiophene-2-sulphonamide

The title compound was prepared starting from amine (IIa-29) and sulphonyl chloride (IIb-22). MS: 467 [M+H⁺]

### Compound 269. 6-Chloro-N-[[1-[2-(1-naphthyloxy)ethyl]pyrrolidin-3-yl]methyl]-benzothiophene-2-sulphonamide

The title compound was prepared starting from amine (IIIa-29) and sulphonyl chloride (IIb-24). MS: 501 [M+H⁺]

### Compound 270. 6-Chloro-N-[[1-[2-(1-naphthyloxy)ethyl]pyrrolidin-3-yl]methyl]naphthalene-2-sulphonamide

The title compound was prepared starting from amine (IIa-29) and sulphonyl chloride (IIb-37). MS: 495 [M+H⁺]

### Compound 271. 5-Fluoro-3-methyl-N-[[1-[2-(1-naphthyloxy)ethyl]pyrrolidin-3-yl]methyl]benzothiophene-2-sulphonamide

The title compound was prepared starting from amine (IIa-29) and sulphonyl chloride (IIb-25). MS: 499 [M+H⁺]

### Compound 272. 5-Chloro-3-methyl-N-[[1-[2-(1-naphthyloxy)ethyl]pyrrolidin-3-yl]methyl]benzothiophene-2-sulphonamide

The title compound was prepared starting from amine (IIa-29) and sulphonyl chloride (IIb-26). MS: 515 [M+H⁺]

### Compound 273. N-[[1-[2-(1-Naphthyloxy)ethyl]pyrrolidin-3-yl]methyl]naphthalene-1-sulphonamide

The title compound was prepared starting from amine (IIa-29) and sulphonyl chloride (IIb-17). MS: 461 [M+H⁺]

### Compound 274. 1-Methyl-N-[[1-[2-(1-naphthyloxy)ethyl]pyrrolidin-3-yl]methyl]indole-5-sulphonamide

The title compound was prepared starting from amine (IIa-29) and sulphonyl chloride (IIb-51). MS: 464 [M+H⁺]

### Compound 275. 1-Methyl-N-[[1-[2-(1-naphthyloxy)ethyl]pyrrolidin-3-yl]methyl]indole-4-sulphonamide

The title compound was prepared starting from amine (IIa-29) and sulphonyl chloride (IIb-50). MS: 464 [M+H⁺]

### Compound 276. N-[[1-[2-(1-Naphthyloxy)ethyl]pyrrolidin-3-yl]methyl]benzothiophene-3-sulphonamide

The title compound was prepared starting from amine (IIa-29) and sulphonyl chloride (IIb-23). MS: 467 [M+H⁺]

### Compound 277. 3-Chloro-4-fluoro-N-[[1-[2-(1-naphthyloxy)ethyl]pyrrolidin-3-yl]methyl]benzenesulphonamide

The title compound was prepared starting from amine (IIIa-29) and sulphonyl chloride (IIb-7). MS: 463 [M+H⁺]

### Compound 278. 3,4-Difluoro-N-[[1-[2-(1-naphthyloxy)ethyl]pyrrolidin-3-yl]methyl]benzenesulphonamide

The title compound was prepared starting from amine (IIa-29) and sulphonyl chloride (IIb-31). MS: 447 [M+H⁺]

### Compound 279. N-[[1-[2-(2,3-Dihydro-1,4-benzodioxin-5-yloxy)ethyl]pyrrolidin-3-yl]methyl]-1,3-benzodioxole-5-sulphonamide

The title compound was prepared starting from amine (IIa-38) and sulphonyl chloride (IIb-48). MS: 463 [M+H⁺]

### Compound 280. N-[[1-[2-(2,3-Dihydro-1,4-benzodioxin-5-yloxy)ethyl]pyrrolidin-3-yl]methyl]benzothiophene-2-sulphonamide

The title compound was prepared starting from amine (IIa-38) and sulphonyl chloride (IIb-22). MS: 475 [M+H⁺]

### Compound 281. N-[[1-[2-(2,3-Dihydro-1,4-benzodioxin-5-yloxy)ethyl]pyrrolidin-3-yl]methyl]-1,3-benzothiazole-4-sulphonamide

The title compound was prepared starting from amine (IIa-38) and sulphonyl chloride (IIb-28), MS: 476 [M+H⁺]

### Compound 282. 6-Chloro-N-[[1-[2-(2,3-dihydro-1,4-benzodioxin-5-yloxy)ethyl]pyrrolidin-3-yl]methyl]naphthalene-2-sulphonamide

The title compound was prepared starting from amine (IIa-38) and sulphonyl chloride (IIb-37). MS: 503 [M+H⁺]

### Compound 283. 5-Chloro-N-[[1-[2-(2,3-dihydro-1,4-benzodioxin-5-yloxy)ethyl]pyrrolidin-3-yl]methyl]-3-methyl-benzothiophene-2-sulphonamide

The title compound was prepared starting from amine (IIa-38) and sulphonyl chloride (IIb-26). MS: 523 [M+H⁺]

### Compound 284. N-[[1-[2-(2,3-Dihydro-1,4-benzodioxin-5-yloxy)ethyl]pyrrolidin-3-yl]methyl]thiazole-2-sulphonamide

The title compound was prepared starting from amine (IIa-38) and sulphonyl chloride (IIb-46). MS: 426 [M+H⁺]

### Compound 285. N-[[1-[2-(2,3-Dihydro-1,4-benzodioxin-5-yloxy)ethyl]pyrrolidin-3-yl]methyl]-1,3-benzothiazole-5-sulphonamide

The title compound was prepared starting from amine (IIa-38) and sulphonyl chloride (IIb-55). MS: 476 [M+H⁺]

### Compound 286. N-[[1-[2-(2,3-Dihydro-1,4-benzodioxin-5-yloxy)ethyl]pyrrolidin-3-yl]methyl]naphthalene-1-sulphonamide

The title compound was prepared starting from amine (IIa-38) and sulphonyl chloride (IIb-17). MS: 469 [M+H⁺]

### Compound 287. N-[[1-[2-(2,3-Dihydro-1,4-benzodioxin-5-yloxy)ethyl]pyrrolidin-3-yl]methyl]naphthalene-2-sulphonamide

The title compound was prepared starting from amine (IIa-38) and sulphonyl chloride (IIb-18). MS: 469 [M+W]

### Compound 288. N-[[1-[2-(2,3-Dihydro-1,4-benzodioxin-5-yloxy)ethyl]pyrrolidin-3-yl]-methyl]-4-phenyl-benzenesulphonamide

The title compound was prepared starting from amine (IIa-38) and sulphonyl chloride (IIb-36). MS: 495 [M+H⁺]

### Compound 289. 4-Chloro-N-[[1-[2-(2,3-dihydro-1,4-benzodioxin-5-yloxy)ethyl]pyrrolidin-3-yl]methyl]benzenesulphonamide

The title compound was prepared starting from amine (IIa-38) and sulphonyl chloride (IIb-5). MS: 453 [M+H⁺]

### Compound 290. 3-Chloro-N-[[1-[2-(2,3-dihydro-1,4-benzodioxin-5-yloxy)ethyl]pyrrolidin-3-yl]methyl]benzenesulphonamide

The title compound was prepared starting from amine (IIa-38) and sulphonyl chloride (IIb-4). MS: 453 [M+H⁺]

### Compound 291. N-[[1-[2-(2,3-Dihydro-1,4-benzodioxin-5-yloxy)ethyl]pyrrolidin-3-yl]methyl]-4-methyl-benzenesulphonamide

The title compound was prepared starting from amine (IIa-38) and sulphonyl chloride (IIb-33). MS: 433 [M+H⁺]

### Compound 292. N-[[1-[2-(2,3-Dihydro-1,4-benzodioxin-5-yloxy)ethyl]pyrrolidin-3-yl]methyl]benzenesulphonamide

The title compound was prepared starting from amine (IIa-38) and sulphonyl chloride (IIb-1). MS: 419 [M+H⁺]

### Compound 293. N-[[1-[2-(2,3-Dihydro-1,4-benzodioxin-5-yloxy)ethyl]pyrrolidin-3-yl]methyl]-4-(trifluoromethyl)benzenesulphonamide

The title compound was prepared starting from amine (IIa-38) and sulphonyl chloride (IIb-12). MS: 487 [M+H⁺]

### Compound 294. N-[[1-[2-(2,3-Dihydro-1,4-benzodioxin-5-yloxy)ethyl]pyrrolidin-3-yl]-methyl]-3-(trifluoromethyl)benzenesulphonamide

The title compound was prepared starting from amine (IIa-38) and sulphonyl chloride (IIb-11). MS: 487 [M+H⁺]

### Compound 295. 4-tert-Butyl-N-[[1-[2-(2,3-dihydro-1,4-benzodioxin-5-yloxy)ethyl]-pyrrolidin-3-yl]methyl]benzenesulphonamide

The title compound was prepared starting from amine (IIa-38) and sulphonyl chloride (IIb-10). MS: 475 [M+H⁺]

### Compound 296. N-[[1-[2-(2,3-Dihydro-1,4-benzodioxin-5-yloxy)ethyl]pyrrolidin-3-yl]methyl]-3-methyl-benzenesulphonamide

The title compound was prepared starting from amine (IIa-38) and sulphonyl chloride (IIb-8). MS: 433 [M+H⁺]

### Compound 297. N-[[1-[2-(2,3-Dihydro-1,4-benzodioxin-5-yloxy)ethyl]pyrrolidin-3-yl]-methyl]-3-methoxybenzenesulphonamide

The title compound was prepared starting from amine (IIa-38) and sulphonyl chloride (IIb-13). MS: 449 [M+H⁺]

### Compound 298. N-[[1-[2-(2,3-Dihydro-1,4-benzodioxin-5-yloxy)ethyl]pyrrolidin-3-yl]-methyl]-3-fluoro-benzenesulphonamide

The title compound was prepared starting from amine (IIa-38) and sulphonyl chloride (IIb-2). MS: 437 [M+H⁺]

### Compound 299. 4-Cyano-N-[[1-[2-(2,3-dihydro-1,4-benzodioxin-5-yloxy)ethyl]pyrrolidin-3-yl]methyl]benzenesulphonamide

The title compound was prepared starting from amine (IIa-38) and sulphonyl chloride (IIb-16). MS: 444 [M+H⁺]

### Compound 300. N-[[1-[2-(2,3-Dihydro-1,4-benzodioxin-5-yloxy)ethyl]pyrrolidin-3-yl]-methyl]-4-fluoro-benzenesulphonamide

The title compound was prepared starting from amine (IIa-38) and sulphonyl chloride (IIb-3). MS: 437 [M+H⁺]

### Compound 301. 3,4-Dichloro-N-[[1-[2-(2,3-dihydro-1,4-benzodioxin-5-yloxy)ethyl]-pyrrolidin-3-yl]methyl]benzenesulphonamide

The title compound was prepared starting from amine (IIa-38) and sulphonyl chloride (IIb-32). MS: 487 [M+H⁺]

### Compound 302. N-[[1-[2-(2,3-Dihydro-1,4-benzodioxin-5-yloxy)ethyl]pyrrolidin-3-yl]-methyl]thiophene-2-sulphonamide

The title compound was prepared starting from amine (IIa-38) and sulphonyl chloride (IIb-40). MS: 425 [M+H⁺]

### Compound 303. N-[[1-[2-(2,3-Dihydro-1,4-benzodioxin-5-yloxy)ethyl]pyrrolidin-3-yl]-methyl]-3-hydroxy-benzenesulphonamide

The title compound was prepared starting from amine (IIa-38) and sulphonyl chloride (IIb-14). MS: 435 [M+H⁺]

### Compound 304. N-[[1-[2-(2,3-Dihydro-1,4-benzodioxin-5-yloxy)ethyl]pyrrolidin-3-yl]-methyl]-4-methoxy-benzenesulphonamide

The title compound was prepared starting from amine (IIa-38) and sulphonyl chloride (IIIb-34). MS: 449 [M+H⁺]

### Compound 305. 4-Bromo-N-[[1-[2-(2,3-dihydro-1,4-benzodioxin-5-yloxy)ethyl]pyrrolidin-3-yl]methyl]benzenesulphonamide

The title compound was prepared starting from amine (IIa-38) and sulphonyl chloride (IIb-6). MS: 497 [M+H⁺]

### Compound 306. N-[[1-[2-(2,3-Dihydro-1,4-benzodioxin-5-yloxy)ethyl]pyrrolidin-3-yl]-methyl]-2,3-dihydrobenzofuran-5-sulphonamide

The title compound was prepared starting from amine (IIIa-38) and sulphonyl chloride (IIb-21). MS: 461 [M+H⁺]

### Compound 307. N-[[1-[2-(2,3-Dihydro-1,4-benzodioxin-5-yloxy)ethyl]pyrrolidin-3-yl]methyl]benzofuran-2-sulphonamide

The title compound was prepared starting from amine (IIa-38) and sulphonyl chloride (IIb-52). MS: 459 [M+H⁺]

### Compound 308. N-[[1-[2-(2,3-Dihydro-1,4-benzodioxin-5-yloxy)ethyl]pyrrolidin-3-yl]methyl]-1-methyl-indole-5-sulphonamide

The title compound was prepared starting from amine (IIa-38) and sulphonyl chloride (IIb-51). MS: 472 [M+H⁺]

### Compound 309. N-[[1-[2-(2,3-Dihydro-1,4-benzodioxin-5-yloxy)ethyl]pyrrolidin-3-yl]methyl]-1-methyl-indole-4-sulphonamide

The title compound was prepared starting from amine (IIa-38) and sulphonyl chloride (IIb-50). MS: 472 [M+H⁺]

### Compound 310. N-[[1-[2-(2,3-Dihydro-1,4-benzodioxin-5-yloxy)ethyl]pyrrolidin-3-yl]methyl]-2-oxo-indoline-5-sulphonamide

The title compound was prepared starting from amine (IIa-38) and sulphonyl chloride (IIb-47). MS: 474 [M+H⁺]

### Compound 311. N-[[1-[2-(2,3-Dihydro-1,4-benzodioxin-5-yloxy)ethyl]pyrrolidin-3-yl]methyl]benzothiophene-3-sulphonamide

The title compound was prepared starting from amine (IIa-38) and sulphonyl chloride (IIb-23). MS: 475 [M+H⁺]

### Compound 312. N-[[1-[2-(2,3-Dihydro-1,4-benzodioxin-5-yloxy)ethyl]pyrrolidin-3-yl]methyl]-2,5-dimethylthiophene-3-sulphonamide

The title compound was prepared starting from amine (IIa-38) and sulphonyl chloride (IIb-42). MS: 453 [M+H⁺]

### Compound 313. N-[[1-[2-(2,3-Dihydro-1,4-benzodioxin-5-yloxy)ethyl]pyrrolidin-3-yl]methyl]thiophene-3-sulphonamide

The title compound was prepared starting from amine (IIa-38) and sulphonyl chloride (IIb-41). MS: 425 [M+H⁺]

### Compound 314. N-[[1-[2-(2,3-Dihydro-1,4-benzodioxin-5-yloxy)ethyl]pyrrolidin-3-yl]methyl]-5-isoxazol-5-yl-thiophene-2-sulphonamide

The title compound was prepared starting from amine (IIa-38) and sulphonyl chloride (IIb-43). MS: 492 [M+H⁺]

### Compound 315. 3-Cyano-N-[[1-[2-(2,3-dihydro-1,4-benzodioxin-5-yloxy)ethyl]pyrrolidin-3-yl]methyl]benzenesulphonamide

The title compound was prepared starting from amine (IIa-38) and sulphonyl chloride (IIb-15). MS: 444 [M+H⁺]

### Compound 316. 5-Chloro-N-[[1-[2-(2,3-dihydro-1,4-benzodioxin-5-yloxy)ethyl]pyrrolidin-3-yl]-methyl]thiophene-2-sulphonamide

The title compound was prepared starting from amine (IIa-38) and sulphonyl chloride (IIb-20). MS: 459 [M+H⁺]

### Compound 317. 3-Chloro-N-[[1-[2-(2,3-dihydro-1,4-benzodioxin-5-yloxy)ethyl]pyrrolidin-3-yl]methyl]-4-fluorobenzenesulphonamide

The title compound was prepared starting from amine (IIa-38) and sulphonyl chloride (IIb-7). MS: 471 [M+H⁺]

### Compound 318. N-[[1-[2-(2,3-Dihydro-1,4-benzodioxin-5-yloxy)ethyl]pyrrolidin-3-yl]methyl]-4-propyl-benzenesulphonamide

The title compound was prepared starting from amine (IIa-38) and sulphonyl chloride (IIb-9). MS: 461 [M+H⁺]

### Compound 319. N-[[1-[2-(2,3-Dihydro-1,4-benzodioxin-5-yloxy)ethyl]pyrrolidin-3-yl]methyl]-3,4-difluorobenzenesulphonamide

The title compound was prepared starting from amine (IIa-38) and sulphonyl chloride (IIb-31). MS: 455 [M+H⁺]

### Compound 320. N-[[1-[2-(2,3-Dihydro-1,4-benzodioxin-5-yloxy)ethyl]pyrrolidin-3-yl]methyl]-4-(trifluoromethoxy)benzenesulphonamide

The title compound was prepared starting from amine (IIa-38) and sulphonyl chloride (IIb-35). MS: 503 [M+H⁺]

### Compound 321. N-[[1-[2-(2,3-Dihydro-1,4-benzodioxin-5-yloxy)ethyl]pyrrolidin-3-yl]methyl]-4-iodo-benzenesulphonamide

The title compound was prepared starting from amine (IIa-38) and sulphonyl chloride (IIb-30). MS: 545 [M+H⁺]

### Compound 322. 3-Bromo-N-[[1-[2-(2,3-dihydro-1,4-benzodioxin-5-yloxy)ethyl]pyrrolidin-3-yl]methyl]benzenesulphonamide

The title compound was prepared starting from amine (IIa-38) and sulphonyl chloride (IIb-29). MS: 497 [M+H⁺]

### Compound 323. N-[[1-[2-(2,3-Dihydro-1,4-benzodioxin-5-yloxy)ethyl]pyrrolidin-3-yl]methyl]-5-methyl-isoxazole-4-sulphonamide

The title compound was prepared starting from amine (IIa-38) and sulphonyl chloride (IIb-45). MS: 424 [M+H⁺]

### Compound 324. N-[1-[3-(6-Fluoro-1,2-benzoxazol-3-yl)propyl]-4-piperidyl]benzothiophene-2-sulphonamide

The title compound was prepared starting from amine (IIa-3) and sulphonyl chloride (IIb-22). MS: 474 [M+H⁺]

### Compound 325. 6-Chloro-N-[1-[3-(6-fluoro-1,2-benzoxazol-3-yl)propyl]-4-piperidyl]naphthalene-2-sulphonamide

The title compound was prepared starting from amine (IIa-3) and sulphonyl chloride (IIb-37). MS: 502 [M+H⁺]

### Compound 326. 5-Chloro-N-[1-[3-(6-fluoro-1,2-benzoxazol-3-yl)propyl]-4-piperidyl]-3-methyl-benzothiophene-2-sulphonamide

The title compound was prepared starting from amine (IIa-3) and sulphonyl chloride (IIb-26). MS: 522 [M+H⁺]

### Compound 327. N-[1-[3-(6-Fluoro-1,2-benzoxazol-3-yl)propyl]-4-piperidyl]-4-phenylbenzenesulphonamide

The title compound was prepared starting from amine (IIa-3) and sulphonyl chloride (IIb-36). MS: 494 [M+H⁺]

### Compound 328. 4-tert-Butyl-N-[1-[3-(6-fluoro-1,2-benzoxazol-3-yl)propyl]-4-piperidyl]benzenesulphonamide

The title compound was prepared starting from amine (IIa-3) and sulphonyl chloride (IIb-10). MS: 474 [M+H⁺]

### Compound 329. N-[1-[3-(6-Fluoro-1,2-benzoxazol-3-yl)propyl]-4-piperidyl]-1-methyl-indole-4-sulphonamide

The title compound was prepared starting from amine (IIa-3) and sulphonyl chloride (IIb-50). MS: 471 [M+H⁺]

### Compound 330. N-[1-[3-(6-Fluoro-1,2-benzoxazol-3-yl)propyl]-4-piperidyl]benzothiophene-3-sulphonamide

The title compound was prepared starting from amine (IIa-3) and sulphonyl chloride (IIb-23). MS: 474 [M+H⁺]

### Compound 331. N-[1-[3-(5-chloro-1H-indol-3-yl)propyl]pyrrolidin-3-yl]benzothiophene-2-sulphonamide

The title compound was prepared starting from amine (IIa-16) and sulphonyl chloride (IIb-22). MS: 474 [M+H⁺]

### Compound 332. 6-chloro-N-[1-[3-(5-chloro-1H-indol-3-yl)propyl]pyrrolidin-3-yl]benzothiophene-2-sulphonamide

The title compound was prepared starting from amine (IIa-16) and sulphonyl chloride (IIb-24). MS: 508 [M+H⁺]

### Compound 333. 6-chloro-N-[1-[3-(5-chloro-1H-indol-3-yl)propyl]pyrrolidin-3-yl]naphthalene-2-sulphonamide

The title compound was prepared starting from amine (IIa-16) and sulphonyl chloride (IIb-37). MS: 502 [M+H⁺]

### Compound 334. N-[1-[3-(5-chloro-1H-indol-3-yl)propyl]pyrrolidin-3-yl]-5-fluoro-3-methylbenzothiophene-2-sulphonamide

The title compound was prepared starting from amine (IIa-16) and sulphonyl chloride (IIb-25). MS: 506 [M+H⁺]

### Compound 335. 5-chloro-N-[1-[3-(5-chloro-1H-indol-3-yl)propyl]pyrrolidin-3-yl]-3-methylbenzothiophene-2-sulphonamide

The title compound was prepared starting from amine (IIa-16) and sulphonyl chloride (IIb-26). MS: 522 [M+H⁺]

### Compound 336. 3,4-dichloro-N-[1-[3-(5-chloro-1H-indol-3-yl)propyl]pyrrolidin-3-yl]benzenesulphonamide

The title compound was prepared starting from amine (IIIa-16) and sulphonyl chloride (IIb-32). MS: 486 [M+H⁺]

### Compound 337. N-[1-[3-(5-chloro-1H-indol-3-yl)propyl]pyrrolidin-3-yl]thiophene-2-sulphonamide

The title compound was prepared starting from amine (IIa-16) and sulphonyl chloride (IIb-40). MS: 424 [M+H⁺]

### Compound 338. N-[1-[3-(5-chloro-1H-indol-3-yl)propyl]pyrrolidin-3-yl]-1-methyl-indole-5-sulphonamide

The title compound was prepared starting from amine (IIa-16) and sulphonyl chloride (IIb-51). MS: 471 [M+H⁺]

### Compound 339. N-[1-[3-(5-chloro-1H-indol-3-yl)propyl]pyrrolidin-3-yl]-1-methyl-indole-4-sulphonamide

The title compound was prepared starting from amine (IIa-16) and sulphonyl chloride (IIb-50). MS: 471 [M+H⁺]

### Compound 340. N-[1-[3-(5-chloro-1H-indol-3-yl)propyl]pyrrolidin-3-yl]benzothiophene-3-sulphonamide

The title compound was prepared starting from amine (IIa-16) and sulphonyl chloride (IIb-23). MS: 474 [M+H⁺]

### Compound 341. N-[1-[3-(5-chloro-H-indol-3-yl)propyl]pyrrolidin-3-yl]naphthalene-1-sulphonamide

The title compound was prepared starting from amine (IIa-16) and sulphonyl chloride (IIb-17). MS: 468 [M+H⁺]

### Compound 342. 3-chloro-N-[1-[3-(5-chloro-1H-indol-3-yl)propyl]pyrrolidin-3-yl]-4-fluorobenzenesulphonamide

The title compound was prepared starting from amine (IIa-16) and sulphonyl chloride (IIb-7). MS: 470 [M+H⁺]

### Compound 343. N-[1-[3-(5-chloro-1H-indol-3-yl)propyl]pyrrolidin-3-yl]-3,4-difluorobenzenesulphonamide

The title compound was prepared starting from amine (IIa-16) and sulphonyl chloride (IIb-31). MS: 454 [M+H⁺]

### Compound 344. N-[1-[3-(5-fluoro-1H-indol-3-yl)propyl]pyrrolidin-3-yl]benzothiophene-2-sulphonamide

The title compound was prepared starting from amine (IIa-15) and sulphonyl chloride (IIb-22). MS: 458 [M+H⁺]

### Compound 345. 6-chloro-N-[1-[3-(5-fluoro-1H-indol-3-yl)propyl]pyrrolidin-3-yl]benzothiophene-2-sulphonamide

The title compound was prepared starting from amine (IIa-15) and sulphonyl chloride (IIb-24). MS: 492 [M+H⁺]

### Compound 346. 6-chloro-N-[1-[3-(5-fluoro-1H-indol-3-yl)propyl]pyrrolidin-3-yl]naphthalene-2-sulphonamide

The title compound was prepared starting from amine (IIa-15) and sulphonyl chloride (IIb-37). MS: 486 [M+H⁺]

### Compound 347. 5-fluoro-N-[1-[3-(5-fluoro-1H-indol-3-yl)propyl]pyrrolidin-3-yl]-3-methylbenzothiophene-2-sulphonamide

The title compound was prepared starting from amine (IIa-15) and sulphonyl chloride (IIb-25). MS: 490 [M+H⁺]

### Compound 348. 5-chloro-N-[1-[3-(5-fluoro-1H-indol-3-yl)propyl]pyrrolidin-3-yl]-3-methylbenzothiophene-2-sulphonamide

The title compound was prepared starting from amine (IIIa-15) and sulphonyl chloride (IIb-26). MS: 506 [M+H⁺]

### Compound 349. 3,4-dichloro-N-[1-[3-(5-fluoro-1H-indol-3-yl)propyl]pyrrolidin-3-yl]benzenesulphonamide

The title compound was prepared starting from amine (IIa-15) and sulphonyl chloride (IIb-32). MS: 470 [M+H⁺]

### Compound 350. N-[1-[3-(5-fluoro-1H-indol-3-yl)propyl]pyrrolidin-3-yl]thiophene-2-sulphonamide

The title compound was prepared starting from amine (IIa-15) and sulphonyl chloride (IIb-40). MS: 408 [M+H⁺]

### Compound 351. N-[1-[3-(5-fluoro-1H-indol-3-yl)propyl]pyrrolidin-3-yl]-1-methyl-indole-5-sulphonamide

The title compound was prepared starting from amine (IIa-15) and sulphonyl chloride (IIb-51). MS: 455 [M+H⁺]

### Compound 352. N-[1-[3-(5-fluoro-1H-indo-3-yl)propyl]pyrrolidin-3-yl]-1-methyl-indole-4-sulphonamide

The title compound was prepared starting from amine (IIa-15) and sulphonyl chloride (IIb-50). MS: 455 [M+H⁺]

### Compound 353. N-[1-[3-(5-fluoro-1H-indol-3-yl)propyl]pyrrolidin-3-yl]benzothiophene-3-sulphonamide

The title compound was prepared starting from amine (IIa-15) and sulphonyl chloride (IIb-23). MS: 457 [M+H⁺]

### Compound 354. 3-chloro-4-fluoro-N-[1-[3-(5-fluoro-1H-indol-3-yl)propyl]pyrrolidin-3-yl]benzenesulphonamide

The title compound was prepared starting from amine (IIa-15) and sulphonyl chloride (IIb-7). MS: 454 [M+H⁺]

### Compound 355. 3,4-difluoro-N-[1-[3-(5-fluoro-1H-indol-3-yl)propyl]pyrrolidin-3-yl]benzenesulphonamide

The title compound was prepared starting from amine (IIa-15) and sulphonyl chloride (IIb-31). MS: 438 [M+H⁺]

### EXAMPLE 3

### In Vitro Pharmacology: Binding Assays

The affinity of compounds of the present invention for dopaminergic, serotoninergic, adrenergic, muscarinic M3, histaminergic H1, and sigma receptors and to serotonin transporter SERT was tested using the methods as described below, by measurement their binding to these receptors using radioreceptors methods. Moreover, the ability of the compounds of the invention to block potassium channel hERG was tested.

The specific ligand binding to the receptors is defined as the difference between the total binding and the non-specific binding determined in the presence of the excess of unlabelled ligand.

The compounds were tested for their affinity to receptors at a concentration of 1×10⁻⁶ M, and for ability to block potassium channel hERG at a concentration of 1×10⁻⁵M.

The results are expressed as a percent of control specific binding ((measured specific binding/control specific binding)×100) and as a percent inhibition of control specific binding (100−((measured specific binding/control specific binding)×100)) obtained in the presence of the test compounds. The specific ligand binding to the receptors is defined as the difference between the total binding and the nonspecific binding determined in the presence of an excess of unlabelled ligand. Scintillation counting was the method of detection of ligand binding. The IC50 values (concentration causing a half-maximal inhibition of control specific binding) were determined by non-linear regression analysis of the competition curves generated with mean replicate values using Hill equation curve fitting (Y=D+[(A−D)/(1+(C/C50)nH)], where Y=specific binding, D=minimum specific binding, A=maximum specific binding, C=compound concentration, C50=IC50, and nH=slope factor). This analysis was performed using a software developed at Cerep (Hill software) and validated by comparison with data generated by the commercial software SigmaPlot® 4.0 for Windows® (© 1997 by SPSS Inc.). The inhibition constants (Ki) were calculated using the Cheng Prusoff equation (Ki=IC50/(1+(L/KD)), where L=concentration of radioligand in the assay, and KD=affinity of the radioligand for the receptor). A scatchard plot was used to determine the Kd.

Conditions and methodology of in vitro tests are given by reference to the literature.

### Affinity for Dopaminergic Receptors D2, D3 and D4

Experimental conditions for tests are given in Table 1, the results of tests for representative compounds are given in Tables 2a and 2b (receptors D2 and D3) and in Table 3 (receptors D4).

TABLE 1
Experimental conditions for testing the affinity for dopaminergic receptors

| | D2 | D3 | D4 |
|---|---|---|---|
| Biological | human recombinant | human recombinant | human recombinant |
| material | (Invitrogen, GeneBLAzer ® | receptors (Membrane | (CHO cells) |
| | D2-Gqo5 CHO-K1 DA) | Target Systems ™ human | |
| | | dopamine D3 Receptor, | |
| | | PerkinElmer) | |
| Radioligand | [3H]methylspiperone | [3H]methylspiperone | [³H]methylspiperone |
| Concentration | about 0.5 nM | 0.3 nM | 0.3 nM |
| Kd | 0.4 nM | 0.1 nM | 0.19 nM |
| Non-specific | haloperidol (1 μM) | (+)-butaclamol (1 μM) | (+)-butaclamol (10 μM) |
| binding | | | |
| Incubation | 60 min, 30° C. | 60 min, 24° C. | 60 min, 22° C. |
| Methodology | Bryan L. Roth. Assay | Missale et al. (1998), | Van Tol, H. H. M et |
| | Protocol Book. University of | Physiol. Rev., 78: 189- | al. (1992) Nature, |
| | North Carolina At Chapel | 225 | 358: 149-152 |
| | Hill. National Institute of | | |
| | Mental Health. | | |
| | Psychoactive Drug | | |
| | Screening Program. | | |
| | Available on-line at | | |
| | 31.08.2008: | | |
| | http://pdsp.med.unc.edu/ | | |
| | UNC- | | |
| | CH%20Protocol%20Book.pdf | | |

TABLE 2a
Results of binding assays for receptors D2 and D3
for representative compounds of the invention

| Compound No. | D2 [%] | D3 [%] |
|---|---|---|
| 6 | 70 | 8 |
| 8 | 72 | 18 |
| 9 | 88 | 30 |
| 10 | 99 | 49 |
| 11 | 89 | 49 |
| 14 | 100 | 90 |
| 15 | 75 | 14 |
| 17 | 80 | 20 |
| 18 | 73 | 31 |
| 20 | 95 | 69 |
| 21 | 100 | 96 |
| 22 | 85 | 23 |
| 23 | 100 | 95 |
| 24 | 94 | 52 |
| 25 | 98 | 97 |
| 26 | 98 | 81 |
| 28 | 97 | 96 |
| 29 | 97 | 85 |
| 30 | 62 | −2 |
| 31 | 87 | 27 |
| 32 | 63 | 57 |
| 33 | 54 | 58 |
| 34 | 76 | 58 |
| 35 | 77 | 56 |
| 36 | 57 | 38 |
| 37 | 83 | 66 |
| 38 | 77 | 64 |
| 39 | 86 | 95 |
| 40 | 49 | 39 |
| 43 | 55 | 29 |
| 44 | 76 | 52 |
| 45 | 53 | 87 |
| 46 | 93 | 104 |
| 47 | 83 | 97 |
| 48 | 84 | 104 |
| 49 | 99 | 106 |
| 50 | 96 | 104 |
| 51 | 54 | 103 |
| 52 | 77 | 106 |
| 53 | 74 | 99 |
| 54 | 83 | 105 |
| 55 | 44 | 93 |
| 56 | 34 | 98 |
| 60 | 41 | 102 |
| 61 | 39 | 83 |
| 67 | 57 | 94 |
| 71 | 27 | −1 |
| 75 | 91 | 86 |
| 77 | 93 | 93 |
| 78 | 89 | 98 |
| 79 | 95 | 97 |
| 80 | 96 | 100 |
| 81 | 60 | 86 |
| 82 | 90 | 94 |
| 83 | 44 | 76 |
| 84 | 89 | 99 |
| 85 | 45 | 75 |
| 86 | 93 | 97 |
| 87 | 63 | 86 |
| 88 | −5 | −2 |
| 89 | 24 | 23 |
| 90 | 5 | −7 |
| 91 | 15 | −8 |
| 92 | 49 | 60 |
| 93 | 78 | 75 |
| 94 | 102 | 105 |
| 95 | 5 | 1 |
| 96 | 46 | 56 |
| 97 | 29 | 40 |
| 98 | 62 | 67 |
| 99 | 38 | 78 |
| 100 | 41 | 74 |
| 107 | 89 | 97 |
| 108 | 93 | 95 |
| 109 | 92 | 86 |
| 110 | 74 | 73 |
| 111 | 97 | 87 |
| 113 | 47 | 55 |
| 114 | 49 | 62 |
| 115 | 74 | 97 |
| 116 | 91 | 98 |
| 117 | 24 | 29 |
| 118 | 32 | 20 |
| 119 | 79 | 93 |
| 120 | −33 | 30 |
| 122 | 99 | 100 |
| 123 | 38 | 86 |
| 124 | 94 | 94 |
| 127 | 95 | 79 |
| 130 | 97 | 94 |
| 131 | 97 | 94 |
| 132 | 82 | 60 |
| 133 | 93 | 74 |
| 134 | 96 | 80 |
| 135 | 92 | 94 |
| 136 | 62 | 57 |
| 137 | 76 | 50 |
| 141 | 74 | 58 |
| 142 | 58 | 45 |
| 143 | 80 | 46 |
| 147 | 88 | 74 |
| 149 | 91 | 39 |
| 150 | 53 | 60 |
| 153 | 82 | 61 |
| 155 | 61 | 68 |
| 157 | 81 | 72 |
| 160 | 86 | 87 |
| 161 | 84 | 75 |
| 162 | 61 | 100 |
| 163 | 73 | 99 |
| 164 | 78 | 98 |
| 165 | 66 | 100 |
| 166 | 82 | 101 |
| 167 | 60 | 97 |
| 168 | 64 | 94 |
| 169 | 84 | 100 |
| 170 | 79 | 98 |
| 171 | 72 | 99 |
| 172 | 76 | 100 |
| 173 | 59 | 99 |
| 174 | 25 | 22 |
| 175 | 68 | 78 |
| 176 | 21 | 8 |
| 177 | 13 | 42 |
| 178 | 8 | 25 |
| 179 | 6 | 40 |
| 180 | 23 | 46 |
| 181 | 73 | 77 |
| 182 | 33 | 45 |
| 183 | 92 | 79 |
| 184 | 24 | 39 |
| 185 | 25 | 0 |
| 186 | 77 | 78 |
| 187 | 56 | 48 |
| 188 | 52 | 51 |
| 189 | 71 | 61 |
| 190 | 51 | 62 |
| 191 | 7 | 25 |
| 192 | 42 | 70 |
| 193 | 81 | 55 |
| 194 | 19 | 48 |
| 195 | 93 | 82 |
| 196 | 100 | 94 |
| 197 | 101 | 83 |
| 198 | 99 | 97 |
| 199 | 96 | 88 |
| 200 | 100 | 94 |
| 201 | 99 | 84 |
| 202 | 101 | 96 |
| 203 | 101 | 95 |
| 204 | 98 | 96 |
| 205 | 99 | 99 |
| 206 | 101 | 95 |
| 207 | 101 | 85 |
| 208 | 90 | 49 |
| 209 | 64 | 34 |
| 210 | 64 | 27 |
| 211 | 93 | 63 |
| 213 | 81 | 57 |
| 214 | 97 | 89 |
| 215 | 97 | 85 |
| 216 | 98 | 85 |
| 217 | 96 | 84 |
| 218 | 99 | 95 |
| 219 | 96 | 81 |
| 221 | 97 | 92 |
| 222 | 97 | 74 |
| 223 | 89 | 57 |
| 224 | 98 | 87 |
| 225 | 97 | 84 |
| 226 | 99 | 95 |
| 227 | 97 | 96 |
| 228 | 100 | 97 |
| 229 | 101 | 93 |
| 230 | 99 | 99 |
| 231 | 95 | 91 |
| 234 | 99 | 94 |
| 235 | 31 | 41 |
| 237 | 97 | 89 |
| 238 | 98 | 95 |
| 239 | 96 | 76 |
| 240 | 95 | 75 |
| 241 | 81 | 87 |
| 242 | 55 | 71 |
| 243 | 14 | 47 |
| 245 | −4 | 40 |
| 249 | 4 | 36 |
| 250 | 18 | 55 |
| 252 | 36 | 43 |
| 253 | 87 | 55 |
| 254 | 75 | 34 |
| 256 | 37 | 25 |
| 257 | 92 | 62 |
| 258 | 92 | 43 |
| 259 | 84 | 61 |
| 261 | 88 | 65 |
| 262 | 26 | 44 |
| 263 | 59 | 71 |
| 268 | 54 | 93 |
| 269 | 38 | 89 |
| 270 | 32 | 91 |
| 271 | 45 | 93 |
| 272 | 40 | 88 |
| 273 | 58 | 90 |
| 274 | 62 | 81 |
| 275 | 66 | 94 |
| 276 | 41 | 91 |
| 277 | 34 | 90 |
| 278 | 31 | 94 |
| 285 | 24 | 18 |
| 289 | −19 | 41 |
| 291 | 29 | 33 |
| 293 | 66 | 60 |
| 298 | 52 | 35 |
| 300 | 35 | 30 |
| 301 | −22 | 34 |
| 302 | 34 | 37 |
| 303 | 79 | 52 |
| 305 | 57 | 48 |
| 306 | 51 | 26 |
| 308 | 57 | 43 |
| 312 | 36 | 36 |
| 316 | −2 | 36 |
| 317 | 53 | 49 |
| 324 | 77 | 41 |
| 325 | 91 | 59 |
| 326 | 86 | 61 |
| 327 | 79 | 56 |
| 328 | 64 | 40 |
| 329 | 84 | 68 |
| 330 | 77 | 64 |

TABLE 2b
Inhibition constants Kᵢ for D2 receptors for
representative compounds of the invention

| Compound No. | D2 [nM] |
|---|---|
| 127 | 22.0 |
| 130 | 10.0 |
| 131 | 19.0 |
| 196 | 0.8 |
| 197 | 5.8 |
| 198 | 1.8 |
| 199 | 4.4 |
| 200 | 0.1 |
| 201 | 15.0 |
| 202 | 1.2 |
| 203 | 0.9 |
| 204 | 0.4 |
| 205 | 1.6 |
| 206 | 0.3 |
| 207 | 3.8 |
| 214 | 2.8 |
| 215 | 3.0 |
| 224 | 8.7 |
| 225 | 9.2 |
| 228 | 0.7 |
| 229 | 2.4 |
| 230 | 1.1 |
| 231 | 6.4 |
| 238 | 3.9 |

TABLE 3
Results of binding assay for receptors D4.4
for representative compounds of the invention

| Compound No. | D4.4 [%] |
|---|---|
| 11 | 92 |
| 20 | 100 |
| 21 | 83 |
| 23 | 55 |
| 25 | 84 |
| 26 | 101 |
| 28 | 75 |
| 29 | 16 |
| 34 | 37 |
| 39 | 73 |
| 46 | 72 |
| 47 | 80 |
| 48 | 80 |
| 49 | 82 |
| 50 | 94 |
| 55 | 31 |
| 75 | 76 |
| 77 | 78 |
| 78 | 79 |
| 79 | 92 |
| 80 | 91 |
| 82 | 94 |
| 84 | 94 |
| 86 | 88 |
| 89 | 22 |
| 92 | 35 |
| 93 | 39 |
| 94 | 90 |
| 107 | 83 |
| 108 | 85 |
| 109 | 69 |
| 110 | 42 |
| 111 | 61 |
| 115 | 55 |
| 116 | 64 |
| 122 | 63 |
| 124 | 54 |
| 127 | 55 |
| 130 | 67 |
| 131 | 50 |
| 132 | 39 |
| 133 | 88 |
| 134 | 77 |
| 135 | 60 |
| 143 | 80 |
| 181 | 64 |
| 183 | 80 |
| 186 | 55 |
| 196 | 78 |
| 197 | 81 |
| 198 | 48 |
| 199 | 69 |
| 200 | 49 |
| 201 | 50 |
| 202 | 76 |
| 203 | 78 |
| 204 | 71 |
| 205 | 73 |
| 206 | 87 |
| 207 | 85 |
| 214 | 61 |
| 215 | 88 |
| 216 | 87 |
| 217 | 72 |
| 218 | 86 |
| 221 | 93 |
| 222 | 98 |
| 223 | 92 |
| 224 | 101 |
| 225 | 91 |
| 226 | 69 |
| 227 | 88 |
| 228 | 68 |
| 229 | 86 |
| 230 | 88 |
| 231 | 89 |
| 234 | 70 |
| 237 | 48 |
| 238 | 48 |
| 239 | 78 |
| 240 | 57 |
| 241 | 33 |
| 293 | 35 |
| 298 | 48 |
| 300 | 52 |
| 303 | 39 |
| 305 | 52 |
| 306 | 33 |
| 308 | 26 |
| 312 | 52 |
| 317 | 59 |

### Affinity for Serotoninergic Receptors 5-HT1A, 5-HT2A, 5-HT6, 5-HT7 and 5-HT2C

Experimental conditions for tests are given in Table 4, and results of tests for representative compounds of the invention are given in Table 5a and 5b (receptors 5-HT1A, 5-HT2A, 5-HT6 and 5-HT7) and in Table 6 (receptors 5-HT2C).

TABLE 4
Experimental conditions for testing the affinity for serotoninergic receptors

| | 5-HT1A | 5-HT2A | 5-HT2C | 5-HT6 | 5-HT7 |
|---|---|---|---|---|---|
| Biological | rat | human | human recombinant | human | human |
| material | hippocampus | recombinant | (HEK-293 cells) | recombinant | recombinant |
| | | (Membrane | | (Membrane | (Membrane |
| | | Target Systems ™ | | Target Systems ™ | Target |
| | | Human Serotonin | | human | Systems ™ |
| | | 5-HT2A Receptor, | | Serotonin 5-HT6 | human |
| | | PerkinElmer) | | Receptor, | Serotonin 5- |
| | | | | PerkinElmer) | HT7 Receptor, |
| | | | | | PerkinElmer) |
| Radioligand | [3H]8-OH-DPAT | [3H]ketanserin | [³H]mesulergine | [3H]LSD | [3H]LSD |
| Concentration | 0.8-1.0 nM | 1 nM | 1 nM | 2.5 nM | 3 nM |
| Kd | 1.0 nM | 0.95 nM | 0.5 nM | 1.9 nM | 2.6 nM |
| Non-specific | serotonin | mianserin | RS 102221 | methiothepin | methiothepin |
| binding | (1 μM) | (1 μM) | (10 μM) | (1 μM) | (1 μM) |
| Incubation | 20 min, 37° C. | 60 min, 30° C. | 120 min, 37° C. | 60 min, 30° C. | 120 min, 30° C. |

5-HT1A: Borsini et al. (1995), Naunyn. Sch. Arch. Pharmacol. 352: 276-282

5-HT2A: Bryan L. Roth. Assay Protocol Book. University of North Carolina At Chapel Hill. National Institute of Mental Health. Psychoactive Drug Screening Program. Available on-line at 31 Aug. 2008: http://pdsp.med.unc.edu/UNC-CH%20Protocol%20Book.pdf

5-HT2C: Stam et al. (1994), Eur. J. Pharmacol., 269: 339-348

5-HT6: Bryan L. Roth. Assay Protocol Book. University of North Carolina At Chapel Hill. National Institute of Mental Health. Psychoactive Drug Screening Program. Available on-line at 31 Aug. 2008: http://pdsp.med.unc.edu/UNC-CH %20Protocol%20Book.pdf

5-HT7: Bryan L. Roth. Assay Protocol Book. University of North Carolina At Chapel Hill. National Institute of Mental Health. Psychoactive Drug Screening Program. Available on-line at 31 Aug. 2008: http://pdsp.med.unc.edu/UNC-CH %20Protocol%20Book.pdf

TABLE 5a
Results of binding assays for serotoninergic receptors
for representative compounds of the invention

| Compound | 5-HT1A | 5-HT2A | 5-HT6 | 5-HT7 |
|---|---|---|---|---|
| No. | [%] | [%] | [%] | [%] |
| 6 | 14 | 77 | 44 | 83 |
| 8 | 5 | 97 | 64 | 85 |
| 9 | 12 | 95 | 75 | 90 |
| 10 | 20 | 96 | 85 | 86 |
| 11 | 60 | 100 | 94 | 97 |
| 14 | 24 | 80 | 82 | 76 |
| 15 | 18 | 57 | 83 | 48 |
| 17 | 23 | 94 | 76 | 80 |
| 18 | 18 | 85 | 59 | 94 |
| 20 | 67 | 100 | 97 | 98 |
| 21 | 66 | 100 | 95 | 100 |
| 22 | 0 | 95 | 72 | 85 |
| 23 | 24 | 99 | 97 | 95 |
| 24 | 19 | 95 | 78 | 82 |
| 25 | 74 | 102 | 95 | 98 |
| 26 | 79 | 102 | 98 | 97 |
| 28 | 61 | 101 | 96 | 94 |
| 29 | 70 | 95 | 92 | 52 |
| 30 | 5 | 95 | 77 | 82 |
| 31 | 58 | 84 | 76 | 89 |
| 32 | 37 | 83 | 63 | 92 |
| 33 | 22 | 97 | 91 | 100 |
| 34 | 32 | 98 | 40 | 100 |
| 35 | 28 | 91 | 97 | 98 |
| 36 | 18 | 59 | 75 | 88 |
| 37 | 29 | 56 | 97 | 94 |
| 38 | 25 | 82 | 104 | 94 |
| 39 | 99 | 99 | 92 | 90 |
| 40 | 68 | 102 | 72 | 85 |
| 43 | 83 | 81 | 87 | 71 |
| 44 | 62 | 78 | 81 | 68 |
| 45 | 88 | 75 | 0 | 93 |
| 46 | 101 | 34 | 9 | 100 |
| 47 | 98 | 80 | 45 | 100 |
| 48 | 100 | 88 | 17 | 98 |
| 49 | 100 | 82 | 34 | 100 |
| 50 | 101 | 97 | 43 | 99 |
| 51 | 91 | 67 | 10 | 101 |
| 52 | 90 | 89 | 41 | 90 |
| 53 | 90 | 85 | 54 | 102 |
| 54 | 90 | 88 | 87 | 102 |
| 55 | 96 | 94 | 3 | 96 |
| 56 | 75 | 84 | 36 | 106 |
| 60 | 85 | 76 | 69 | 96 |
| 61 | 77 | 92 | 71 | 95 |
| 67 | 76 | 80 | 87 | 79 |
| 71 | 28 | 26 | 80 | 31 |
| 75 | 100 | 90 | 94 | 90 |
| 77 | 99 | 100 | 95 | 89 |
| 78 | 99 | 95 | 94 | 89 |
| 79 | 99 | 99 | 86 | 96 |
| 80 | 98 | 100 | 96 | 93 |
| 81 | 109 | 75 | 75 | 84 |
| 82 | 99 | 91 | 93 | 96 |
| 83 | 104 | 82 | 84 | 86 |
| 84 | 99 | 78 | 96 | 98 |
| 85 | 102 | 64 | 82 | 89 |
| 86 | 101 | 97 | 98 | 98 |
| 87 | 105 | 78 | 91 | 71 |
| 88 | 96 | 34 | 13 | 84 |
| 89 | 98 | 74 | 19 | 96 |
| 90 | 82 | 49 | −16 | 92 |
| 91 | 88 | 47 | −20 | 91 |
| 92 | 100 | 60 | 23 | 99 |
| 93 | 98 | 72 | 29 | 99 |
| 94 | 98 | 99 | 65 | 100 |
| 95 | 94 | 0 | 9 | 94 |
| 96 | 92 | 93 | 5 | 98 |
| 97 | 84 | 89 | 43 | 105 |
| 98 | 92 | 87 | 32 | 100 |
| 99 | 96 | 97 | 64 | 90 |
| 100 | 94 | 97 | 28 | 103 |
| 107 | 100 | 97 | 39 | 84 |
| 108 | 98 | 98 | 59 | 98 |
| 109 | 96 | 95 | 67 | 99 |
| 110 | 95 | 91 | −60 | 99 |
| 111 | 99 | 92 | 41 | 100 |
| 113 | 81 | 65 | 52 | 85 |
| 114 | 98 | 90 | 56 | 97 |
| 115 | 100 | 99 | 88 | 98 |
| 116 | 99 | 96 | 95 | −49 |
| 117 | 76 | 86 | 34 | 93 |
| 118 | 85 | 83 | 51 | 99 |
| 119 | 89 | 69 | 57 | 85 |
| 120 | 8 | 68 | 24 | 89 |
| 122 | 99 | 83 | 99 | 100 |
| 123 | 99 | 64 | 72 | 88 |
| 124 | 99 | 87 | 99 | 99 |
| 127 | 71 | 99 | 96 | 96 |
| 130 | 85 | 100 | 98 | 98 |
| 131 | 87 | 99 | 97 | 95 |
| 132 | 57 | 97 | 69 | 94 |
| 133 | 52 | 95 | 92 | 97 |
| 134 | 73 | 99 | 94 | 95 |
| 135 | 77 | 99 | 92 | 97 |
| 136 | 35 | 82 | 72 | 80 |
| 137 | 14 | 62 | 14 | 82 |
| 141 | 9 | 85 | 92 | 80 |
| 142 | 29 | 77 | 37 | 87 |
| 143 | 44 | 94 | 78 | 87 |
| 147 | 60 | 95 | 86 | 89 |
| 149 | 82 | 96 | 71 | 113 |
| 150 | −9 | 85 | 65 | 84 |
| 153 | 55 | 96 | 80 | 86 |
| 155 | 42 | 89 | 62 | 82 |
| 157 | 61 | 95 | 80 | 93 |
| 160 | 76 | 97 | 83 | 75 |
| 161 | 86 | 88 | 87 | 83 |
| 162 | 85 | −17 | −56 | 91 |
| 163 | 88 | 99 | 87 | 88 |
| 164 | 83 | 102 | 94 | 92 |
| 165 | 87 | 99 | 97 | 105 |
| 166 | 78 | 100 | 98 | 97 |
| 167 | 75 | 95 | 94 | 90 |
| 168 | 79 | 96 | 93 | 92 |
| 169 | 84 | 69 | 100 | 93 |
| 170 | 90 | 72 | 93 | 104 |
| 171 | 92 | 101 | 96 | 94 |
| 172 | 85 | 71 | 96 | 92 |
| 173 | 83 | 73 | 91 | 105 |
| 174 | 91 | 44 | 64 | 90 |
| 175 | 75 | 105 | 95 | 92 |
| 176 | 98 | 35 | 14 | 87 |
| 177 | 97 | 66 | 44 | 88 |
| 178 | 96 | 73 | 21 | 89 |
| 179 | 92 | 57 | −15 | 90 |
| 180 | 93 | 68 | 31 | 94 |
| 181 | 98 | 89 | 30 | 92 |
| 182 | 63 | 61 | 31 | 95 |
| 183 | 98 | 94 | 81 | 102 |
| 184 | 71 | 76 | 20 | 89 |
| 185 | 88 | 39 | 23 | 90 |
| 186 | 98 | 87 | 32 | 97 |
| 187 | 78 | 93 | 44 | 98 |
| 188 | 101 | 89 | 14 | 91 |
| 189 | 112 | 74 | 46 | 90 |
| 190 | 100 | 68 | 34 | 97 |
| 191 | 98 | 42 | 5 | 93 |
| 192 | 79 | 86 | 30 | 97 |
| 193 | 84 | 88 | 9 | 92 |
| 194 | 63 | 29 | 53 | 82 |
| 195 | 47 | 101 | 86 | 86 |
| 196 | 54 | 100 | 99 | 101 |
| 197 | 56 | 99 | 97 | 101 |
| 198 | 45 | 100 | 97 | 93 |
| 199 | 38 | 99 | 91 | 94 |
| 200 | 50 | 99 | 99 | 93 |
| 201 | 32 | 98 | 94 | 95 |
| 202 | 65 | 99 | 100 | 98 |
| 203 | 60 | 99 | 100 | 99 |
| 204 | 71 | 98 | 97 | 87 |
| 205 | 63 | 99 | 94 | 98 |
| 206 | 68 | 100 | 101 | 100 |
| 207 | 58 | 100 | 101 | 99 |
| 208 | 9 | 100 | 87 | 96 |
| 209 | 11 | 86 | 62 | 90 |
| 210 | 8 | 80 | 68 | 80 |
| 211 | 32 | 100 | 92 | 107 |
| 213 | 63 | 100 | 78 | 93 |
| 214 | 34 | 99 | 97 | 98 |
| 215 | 52 | 99 | 93 | 98 |
| 216 | 35 | 101 | 97 | 99 |
| 217 | 54 | 101 | 98 | 97 |
| 218 | 91 | 100 | 95 | 98 |
| 219 | 85 | 99 | 94 | 96 |
| 221 | 67 | 101 | 92 | 95 |
| 222 | 72 | 99 | 94 | 102 |
| 223 | 49 | 99 | 94 | 100 |
| 224 | 76 | 99 | 99 | 103 |
| 225 | 62 | 99 | 99 | 100 |
| 226 | 43 | 101 | 92 | 91 |
| 227 | 62 | 101 | 94 | 91 |
| 228 | 27 | 99 | 100 | 92 |
| 229 | 46 | 100 | 98 | 99 |
| 230 | 77 | 99 | 96 | 87 |
| 231 | 34 | 100 | 86 | 88 |
| 234 | 45 | 100 | 98 | 97 |
| 235 | 14 | 91 | 62 | 81 |
| 237 | 45 | 97 | 87 | 96 |
| 238 | 71 | 99 | 96 | 94 |
| 239 | 56 | 98 | 97 | 92 |
| 240 | 30 | 99 | 93 | 90 |
| 241 | 39 | 97 | 68 | 100 |
| 242 | 11 | 99 | 60 | 83 |
| 243 | 21 | 90 | 75 | 89 |
| 245 | 35 | 73 | 57 | 81 |
| 249 | 23 | 79 | 58 | 87 |
| 250 | 30 | 87 | 74 | 91 |
| 252 | 44 | 89 | 75 | 90 |
| 253 | 34 | 99 | 101 | 97 |
| 254 | 49 | 98 | 78 | 102 |
| 256 | 29 | 87 | 74 | 92 |
| 257 | 87 | 100 | 94 | 88 |
| 258 | 64 | 98 | 89 | 78 |
| 259 | 42 | 100 | 92 | 91 |
| 261 | 78 | 101 | 97 | 104 |
| 262 | 25 | 85 | 69 | 92 |
| 263 | 19 | 97 | 85 | 92 |
| 268 | 77 | 82 | 84 | 88 |
| 269 | 60 | 91 | 82 | 98 |
| 270 | 47 | 86 | 86 | 94 |
| 271 | 82 | 97 | 76 | 104 |
| 272 | 63 | 82 | 67 | 88 |
| 273 | 85 | 95 | 90 | 77 |
| 274 | 77 | 89 | 97 | 86 |
| 275 | 86 | 91 | 84 | 111 |
| 276 | 81 | 92 | 84 | 82 |
| 277 | 85 | 85 | 93 | 108 |
| 278 | 84 | 91 | 85 | 102 |
| 285 | 62 | 77 | 34 | 86 |
| 289 | 99 | 61 | −1 | 81 |
| 291 | 94 | 63 | 19 | 80 |
| 293 | 100 | 86 | 47 | 90 |
| 298 | 97 | 88 | 40 | 93 |
| 300 | 99 | 72 | 38 | 90 |
| 301 | 96 | 81 | 61 | 98 |
| 302 | 89 | 52 | 35 | 105 |
| 303 | 96 | 96 | 48 | 98 |
| 305 | 101 | 93 | 52 | 96 |
| 306 | 99 | 81 | 45 | 94 |
| 308 | 98 | 91 | 44 | 83 |
| 312 | 98 | 87 | 19 | 86 |
| 316 | 74 | 82 | 36 | 87 |
| 317 | 100 | 72 | 76 | 95 |
| 324 | 49 | 99 | 78 | 101 |
| 325 | 80 | 92 | 72 | 89 |
| 326 | −1 | 97 | 83 | 91 |
| 327 | 20 | 94 | 68 | 86 |
| 328 | 49 | 96 | 94 | 90 |
| 329 | 49 | 94 | 62 | 97 |
| 330 | 82 | 102 | 79 | 101 |

TABLE 5b
Inhibition constants Kᵢ for 5-HT2A and 5-HT6 serotoninergic
receptors for representative compounds of the invention

| Compound No. | 5-HT2A [nM] | 5-HT6 [nM] |
|---|---|---|
| 75 | | 34.0 |
| 77 | | 39.0 |
| 78 | | 42.0 |
| 80 | | 15.0 |
| 82 | | 37.0 |
| 84 | | 18.0 |
| 86 | | 13.0 |
| 116 | | 71.0 |
| 122 | | 13.0 |
| 127 | 4.2 | 18.0 |
| 130 | 4.2 | 9.7 |
| 131 | 5.5 | 9.0 |
| 196 | 0.4 | 6.4 |
| 197 | 0.7 | 16.0 |
| 198 | 0.7 | 8.6 |
| 199 | 0.7 | 99.0 |
| 200 | 2.3 | 10.0 |
| 201 | 7.0 | 79.0 |
| 202 | 1.4 | 7.1 |
| 203 | 0.7 | 12.0 |
| 204 | 0.8 | 6.9 |
| 205 | 1.6 | 41.0 |
| 206 | 0.7 | 6.1 |
| 207 | 0.5 | 7.7 |
| 214 | 2.1 | 7.5 |
| 215 | 1.2 | 21.0 |
| 224 | 1.6 | 4.0 |
| 225 | 0.9 | 1.6 |
| 228 | 1.0 | 14.0 |
| 229 | 0.6 | 43.0 |
| 230 | 0.4 | 15.0 |
| 231 | 1.9 | 79.0 |
| 238 | 3.0 | 17.0 |

TABLE 6
Results of binding assays for serotoninergic 5-HT2C receptors
for representative compounds of the invention

| Compound No. | 5-HT2C [%] |
|---|---|
| 11 | 27 |
| 20 | 33 |
| 21 | 71 |
| 23 | 67 |
| 25 | 83 |
| 26 | 51 |
| 28 | 72 |
| 29 | 28 |
| 34 | 94 |
| 39 | 74 |
| 46 | 21 |
| 47 | 33 |
| 48 | 32 |
| 49 | 59 |
| 50 | 54 |
| 55 | 30 |
| 75 | 83 |
| 77 | 77 |
| 78 | 78 |
| 80 | 86 |
| 82 | 82 |
| 84 | 88 |
| 89 | 53 |
| 92 | 52 |
| 93 | 50 |
| 94 | 91 |
| 107 | 86 |
| 110 | 85 |
| 111 | 87 |
| 122 | 98 |
| 124 | 80 |
| 127 | 84 |
| 130 | 89 |
| 131 | 88 |
| 132 | 43 |
| 133 | 47 |
| 134 | 87 |
| 135 | 86 |
| 143 | 68 |
| 181 | 93 |
| 183 | 98 |
| 186 | 79 |
| 196 | 86 |
| 197 | 84 |
| 198 | 86 |
| 199 | 83 |
| 200 | 77 |
| 201 | 72 |
| 202 | 88 |
| 203 | 90 |
| 204 | 87 |
| 205 | 86 |
| 206 | 84 |
| 214 | 65 |
| 215 | 74 |
| 216 | 62 |
| 217 | 67 |
| 218 | 82 |
| 221 | 81 |
| 222 | 65 |
| 223 | 65 |
| 224 | 71 |
| 225 | 72 |
| 226 | 89 |
| 227 | 82 |
| 228 | 91 |
| 230 | 91 |
| 231 | 75 |
| 234 | 86 |
| 237 | 79 |
| 239 | 49 |
| 240 | 81 |
| 241 | 66 |
| 293 | 50 |
| 298 | 37 |
| 300 | 34 |
| 303 | 47 |
| 305 | 59 |
| 306 | 36 |
| 308 | 61 |
| 312 | 55 |
| 317 | 45 |

### Affinity for Adrenergic α1 and α2C Receptors

Experimental conditions for tests are given in Table 7, and results of tests for representative compounds are given in Tables 8 (α1 receptors) and in Tables 9 (α2C receptors).

TABLE 7
Experimental conditions for testing the affinity for adrenergic receptors

| | α1 | α2C |
|---|---|---|
| Biological | rat cerebral cortex | human recombinant (CHO |
| material | | cells) |
| Radioligand | [3H]prazosina | [³H]RX 821002 |
| Concentration | 0.2 nM | 2 nM |
| Kd | 0.2 nM | 0.95 nM |
| Non-specific | Risperidon (1 μM) | (−)epinephrine |
| binding | | (100 μM) |
| Incubation | 30 min, 30° C. | 60 min, 22° C. |
| Methodology | Leopoldo M et al. (2002), | Devedjian et al. (1994), Eur. |
| | J Med Chem., (26): 5727-35 | J. Pharmacol., 252: 43-49 |

TABLE 8
Results of test of affinity for α1 adrenergic receptors
for representative compounds of the invention

| Compound No. | α1 [%] |
|---|---|
| 6 | 98 |
| 8 | 95 |
| 9 | 83 |
| 10 | 89 |
| 11 | 70 |
| 14 | 87 |
| 15 | 89 |
| 17 | 98 |
| 18 | 98 |
| 20 | 86 |
| 21 | 95 |
| 22 | 91 |
| 23 | 64 |
| 24 | 101 |
| 25 | 85 |
| 26 | 87 |
| 28 | 70 |
| 29 | 18 |
| 30 | 84 |
| 31 | 100 |
| 34 | 91 |
| 35 | 99 |
| 36 | 103 |
| 37 | 96 |
| 38 | 98 |
| 39 | 29 |
| 40 | 12 |
| 43 | 68 |
| 44 | 46 |
| 45 | 72 |
| 46 | 85 |
| 47 | 71 |
| 48 | 87 |
| 49 | 89 |
| 50 | 88 |
| 51 | 77 |
| 52 | 81 |
| 53 | 88 |
| 54 | 90 |
| 55 | 51 |
| 56 | 74 |
| 60 | 87 |
| 61 | 79 |
| 67 | 91 |
| 71 | 36 |
| 75 | 45 |
| 77 | 29 |
| 78 | 39 |
| 79 | 37 |
| 80 | 50 |
| 81 | 36 |
| 82 | 43 |
| 83 | 23 |
| 84 | 51 |
| 85 | 9 |
| 86 | 50 |
| 87 | 18 |
| 88 | −23 |
| 89 | 45 |
| 90 | −25 |
| 91 | −15 |
| 92 | 47 |
| 93 | 52 |
| 94 | 87 |
| 95 | −21 |
| 96 | 60 |
| 97 | 64 |
| 98 | 78 |
| 99 | 70 |
| 100 | 87 |
| 107 | 28 |
| 108 | 40 |
| 109 | 44 |
| 110 | 45 |
| 111 | 68 |
| 113 | 55 |
| 114 | 27 |
| 115 | 24 |
| 116 | 23 |
| 117 | 53 |
| 118 | 72 |
| 119 | 78 |
| 120 | −9 |
| 122 | 53 |
| 123 | 43 |
| 124 | 33 |
| 127 | 93 |
| 130 | 86 |
| 131 | 70 |
| 132 | 86 |
| 133 | 90 |
| 134 | 61 |
| 135 | 88 |
| 143 | 97 |
| 150 | 98 |
| 160 | 49 |
| 161 | 46 |
| 162 | 83 |
| 163 | 27 |
| 164 | 13 |
| 165 | 15 |
| 166 | 23 |
| 167 | 3 |
| 168 | 31 |
| 169 | 74 |
| 170 | 63 |
| 171 | 50 |
| 172 | 40 |
| 173 | 56 |
| 174 | 15 |
| 175 | 50 |
| 176 | 51 |
| 177 | 55 |
| 178 | 20 |
| 179 | 61 |
| 180 | 39 |
| 181 | 50 |
| 182 | 78 |
| 183 | 85 |
| 184 | 88 |
| 185 | 34 |
| 186 | 80 |
| 187 | 88 |
| 188 | 91 |
| 189 | 71 |
| 190 | 86 |
| 191 | 54 |
| 192 | 88 |
| 193 | 71 |
| 194 | 96 |
| 195 | 79 |
| 196 | 95 |
| 197 | 93 |
| 198 | 76 |
| 199 | 66 |
| 200 | 85 |
| 201 | 82 |
| 202 | 98 |
| 203 | 91 |
| 204 | 76 |
| 205 | 71 |
| 206 | 93 |
| 207 | 95 |
| 210 | 94 |
| 211 | 90 |
| 214 | 91 |
| 215 | 89 |
| 216 | 84 |
| 217 | 40 |
| 218 | 96 |
| 221 | 85 |
| 222 | 92 |
| 223 | 86 |
| 224 | 89 |
| 225 | 93 |
| 226 | 73 |
| 227 | 85 |
| 228 | 88 |
| 229 | 89 |
| 230 | 83 |
| 231 | 79 |
| 234 | 92 |
| 237 | 88 |
| 238 | 82 |
| 239 | 91 |
| 240 | 86 |
| 241 | 62 |
| 253 | 94 |
| 268 | 9 |
| 269 | −1 |
| 270 | −5 |
| 271 | −1 |
| 272 | −7 |
| 273 | 25 |
| 274 | 41 |
| 275 | 53 |
| 276 | 16 |
| 277 | 21 |
| 278 | 37 |
| 285 | 69 |
| 289 | 46 |
| 291 | 66 |
| 293 | 77 |
| 298 | 77 |
| 300 | 83 |
| 301 | 46 |
| 302 | 63 |
| 303 | 89 |
| 305 | 84 |
| 306 | 90 |
| 308 | 69 |
| 312 | 65 |
| 316 | 66 |
| 317 | 84 |
| 325 | 76 |
| 326 | 70 |
| 327 | 81 |

TABLE 9
Results of test of affinity for α2C
adrenergic receptors for representative
compounds of the invention

| Compound | |
|---|---|
| No. | α2C [%] |
| 11 | 71 |
| 20 | 78 |
| 21 | 78 |
| 23 | 89 |
| 25 | 90 |
| 26 | 76 |
| 28 | 93 |
| 29 | 84 |
| 34 | 94 |
| 39 | 92 |
| 46 | 101 |
| 47 | 80 |
| 48 | 105 |
| 49 | 110 |
| 50 | 108 |
| 55 | 99 |
| 75 | 76 |
| 77 | 78 |
| 78 | 85 |
| 79 | 96 |
| 80 | 88 |
| 82 | 101 |
| 84 | 105 |
| 86 | 106 |
| 89 | 69 |
| 92 | 76 |
| 93 | 84 |
| 94 | 106 |
| 107 | 77 |
| 108 | 83 |
| 109 | 98 |
| 110 | 98 |
| 111 | 90 |
| 115 | 90 |
| 116 | 93 |
| 122 | 101 |
| 124 | 95 |
| 127 | 93 |
| 130 | 88 |
| 131 | 92 |
| 132 | 73 |
| 133 | 91 |
| 134 | 87 |
| 135 | 95 |
| 143 | 78 |
| 181 | 96 |
| 183 | 99 |
| 186 | 96 |
| 196 | 89 |
| 197 | 90 |
| 198 | 80 |
| 199 | 82 |
| 200 | 96 |
| 201 | 89 |
| 202 | 95 |
| 203 | 93 |
| 204 | 89 |
| 205 | 86 |
| 206 | 93 |
| 207 | 90 |
| 214 | 74 |
| 215 | 66 |
| 216 | 80 |
| 217 | 96 |
| 218 | 94 |
| 221 | 88 |
| 222 | 77 |
| 223 | 60 |
| 224 | 79 |
| 225 | 80 |
| 226 | 86 |
| 227 | 90 |
| 228 | 95 |
| 229 | 98 |
| 230 | 92 |
| 231 | 89 |
| 234 | 90 |
| 237 | 82 |
| 238 | 94 |
| 239 | 89 |
| 240 | 84 |
| 241 | 87 |
| 293 | 95 |
| 298 | 94 |
| 300 | 92 |
| 303 | 100 |
| 305 | 96 |
| 306 | 98 |
| 308 | 96 |
| 312 | 94 |
| 317 | 95 |

### Affinity for Muscarinic M3 Receptors

Experimental conditions for tests are given in Table 10, and results of tests for representative compounds are given in Table 11.

TABLE 10
Experimental conditions for testing the affinity for M3 muscarinic receptors

| | M3 |
|---|---|
| Biological material | human recombinant, (CHO cells) |
| Radioligand | [³H]4-DAMP |
| Concentration | 0.2 nM |
| Kd | 0.5 nM |
| Non-specific binding | atropine (1 μM) |
| Incubation | 60 min, 22° C. |
| Methodology | Peralta et al. (1987), Embo. J., 6: 3923-3929. |

TABLE 11
Results of test of affinity for M3
muscarinic receptors for representative
compounds of the invention

| Compound | |
|---|---|
| No. | M3 [%] |
| 11 | 3 |
| 20 | −7 |
| 21 | −11 |
| 23 | 10 |
| 25 | 23 |
| 26 | 11 |
| 28 | 13 |
| 29 | 30 |
| 34 | 12 |
| 39 | 19 |
| 46 | −13 |
| 47 | -8 |
| 48 | 1 |
| 49 | 11 |
| 50 | 2 |
| 55 | −1 |
| 75 | 17 |
| 77 | 17 |
| 78 | 5 |
| 79 | −3 |
| 80 | 2 |
| 82 | 9 |
| 84 | 3 |
| 86 | 1 |
| 89 | −6 |
| 92 | 4 |
| 93 | 1 |
| 94 | 1 |
| 107 | 8 |
| 108 | −4 |
| 109 | 3 |
| 110 | −7 |
| 111 | 14 |
| 115 | 13 |
| 116 | 9 |
| 122 | 30 |
| 124 | 49 |
| 127 | 1 |
| 130 | 15 |
| 131 | 17 |
| 132 | 10 |
| 133 | 17 |
| 134 | 34 |
| 135 | 19 |
| 143 | −4 |
| 181 | 0 |
| 183 | 13 |
| 186 | −9 |
| 196 | 11 |
| 197 | 26 |
| 198 | 37 |
| 199 | 38 |
| 200 | 3 |
| 201 | 9 |
| 202 | 9 |
| 203 | 12 |
| 204 | 17 |
| 205 | 3 |
| 206 | 9 |
| 207 | 12 |
| 214 | 11 |
| 215 | 39 |
| 216 | 28 |
| 217 | 6 |
| 218 | 0 |
| 221 | 12 |
| 222 | 18 |
| 223 | 25 |
| 224 | 25 |
| 225 | 23 |
| 226 | 8 |
| 227 | 13 |
| 228 | 12 |
| 229 | 15 |
| 230 | 15 |
| 231 | 22 |
| 234 | 17 |
| 237 | 16 |
| 238 | 26 |
| 239 | 23 |
| 240 | 21 |
| 241 | 19 |
| 293 | 10 |
| 298 | 22 |
| 300 | −2 |
| 303 | 9 |
| 305 | 11 |
| 306 | 12 |
| 308 | 13 |
| 312 | 10 |
| 317 | 8 |

### Affinity for Serotonin Transporter (SERT)

Experimental conditions for tests are given in Table 12, and results of tests for representative compounds are given in Tables 13a and 13b.

TABLE 12
Experimental conditions for testing the affinity for
serotonin transporter (SERT)

| | SERT |
|---|---|
| Biological material | human recombinant SERT receptor (CHO cells) |
| Radioligand | [³H]imipramine |
| Concentration | 2 nM |
| Kd | 1.7 nM |
| Non-specific binding | imipramine (10 μM) |
| Incubation | 60 min, 22° C. |
| Methodology | Tatsumi et al. (1999), Eur. J. Pharmacol., |
| | 368: 277-283. |

TABLE 13a
Results of serotonin transporter (SERT) receptor affinity
tests for representative compounds of the invention

| Compound No. | SERT [%] |
|---|---|
| 6 | 18 |
| 8 | −13 |
| 9 | 2 |
| 10 | −2 |
| 11 | −6 |
| 14 | −3 |
| 15 | −7 |
| 17 | 52 |
| 18 | 37 |
| 20 | 6 |
| 21 | 59 |
| 22 | 5 |
| 23 | 53 |
| 24 | −10 |
| 25 | 42 |
| 26 | 66 |
| 28 | 54 |
| 29 | 44 |
| 30 | 36 |
| 31 | 36 |
| 32 | 5 |
| 33 | −4 |
| 34 | 43 |
| 35 | 29 |
| 36 | 0 |
| 37 | 30 |
| 38 | 17 |
| 39 | 29 |
| 40 | −5 |
| 43 | −7 |
| 44 | 25 |
| 45 | −4 |
| 46 | 7 |
| 47 | 6 |
| 48 | −3 |
| 49 | 41 |
| 50 | 20 |
| 51 | 6 |
| 52 | 38 |
| 53 | 56 |
| 54 | 46 |
| 55 | 6 |
| 56 | 15 |
| 60 | 21 |
| 61 | 43 |
| 67 | 38 |
| 71 | 49 |
| 75 | 103 |
| 77 | 104 |
| 78 | 101 |
| 79 | 105 |
| 80 | 104 |
| 81 | 78 |
| 82 | 100 |
| 83 | 78 |
| 84 | 104 |
| 85 | 70 |
| 86 | 105 |
| 87 | 64 |
| 88 | −13 |
| 89 | 3 |
| 90 | 18 |
| 91 | −4 |
| 92 | 10 |
| 93 | 23 |
| 94 | 62 |
| 95 | −4 |
| 96 | 11 |
| 97 | −1 |
| 98 | 16 |
| 99 | 36 |
| 100 | 57 |
| 107 | −4 |
| 108 | 36 |
| 109 | −10 |
| 110 | 39 |
| 111 | 15 |
| 113 | 18 |
| 114 | 5 |
| 115 | 83 |
| 116 | 95 |
| 117 | 33 |
| 118 | 41 |
| 119 | 10 |
| 120 | 21 |
| 122 | 98 |
| 123 | 83 |
| 124 | 100 |
| 127 | 49 |
| 130 | 64 |
| 131 | 55 |
| 132 | 20 |
| 133 | 42 |
| 134 | 47 |
| 136 | 9 |
| 135 | 68 |
| 137 | −5 |
| 141 | −10 |
| 142 | −4 |
| 143 | 6 |
| 147 | 24 |
| 149 | 22 |
| 150 | 12 |
| 153 | 21 |
| 155 | −12 |
| 157 | 25 |
| 160 | 100 |
| 161 | 66 |
| 162 | −9 |
| 163 | 45 |
| 164 | 34 |
| 165 | 111 |
| 166 | 32 |
| 167 | 31 |
| 168 | 25 |
| 169 | 75 |
| 170 | 93 |
| 171 | 44 |
| 172 | 46 |
| 173 | 50 |
| 174 | 20 |
| 175 | 19 |
| 176 | −2 |
| 177 | 25 |
| 178 | −6 |
| 179 | −8 |
| 180 | 14 |
| 181 | 26 |
| 182 | −7 |
| 183 | 48 |
| 184 | 1 |
| 185 | 23 |
| 186 | 39 |
| 187 | 69 |
| 188 | 44 |
| 189 | 41 |
| 190 | 31 |
| 191 | 32 |
| 192 | 38 |
| 193 | 41 |
| 194 | 1 |
| 195 | 7 |
| 196 | 42 |
| 197 | 58 |
| 198 | 44 |
| 199 | 39 |
| 200 | 74 |
| 201 | 59 |
| 202 | 58 |
| 203 | 73 |
| 204 | 70 |
| 205 | 45 |
| 207 | 58 |
| 206 | 62 |
| 208 | −2 |
| 209 | 6 |
| 210 | 35 |
| 211 | 4 |
| 213 | 12 |
| 214 | 15 |
| 215 | 35 |
| 216 | 53 |
| 217 | 90 |
| 218 | 95 |
| 219 | 52 |
| 221 | 67 |
| 222 | 2 |
| 223 | 17 |
| 224 | 64 |
| 225 | 77 |
| 226 | 54 |
| 227 | 69 |
| 228 | 61 |
| 229 | 50 |
| 230 | 75 |
| 231 | 63 |
| 234 | 65 |
| 235 | 11 |
| 237 | 61 |
| 238 | 64 |
| 239 | 36 |
| 240 | 43 |
| 241 | 71 |
| 242 | 37 |
| 243 | 7 |
| 245 | 18 |
| 249 | 28 |
| 250 | 23 |
| 252 | −2 |
| 253 | 12 |
| 254 | 10 |
| 256 | 40 |
| 257 | 20 |
| 258 | 25 |
| 259 | −5 |
| 261 | 3 |
| 262 | 26 |
| 263 | 11 |
| 268 | 85 |
| 269 | 53 |
| 270 | 15 |
| 271 | 40 |
| 272 | 20 |
| 273 | 64 |
| 274 | 109 |
| 275 | 93 |
| 276 | 84 |
| 277 | 68 |
| 278 | 91 |
| 285 | 60 |
| 291 | 66 |
| 289 | 53 |
| 293 | 82 |
| 298 | 72 |
| 300 | 64 |
| 301 | 38 |
| 302 | 37 |
| 303 | 73 |
| 305 | 94 |
| 306 | 90 |
| 308 | 95 |
| 312 | 87 |
| 316 | 8 |
| 317 | 63 |
| 324 | 18 |
| 325 | 21 |
| 326 | 10 |
| 327 | 33 |
| 328 | 25 |
| 329 | 43 |
| 330 | 22 |

TABLE 13b
Inhibition constants Kᵢ for SERT for
representative compounds of the invention

| Compound No. | SERT [nM] |
|---|---|
| 75 | 1.5 |
| 77 | 0.5 |
| 78 | 1.0 |
| 79 | 0.7 |
| 80 | 0.5 |
| 82 | 1.3 |
| 84 | 2.6 |
| 86 | 1.1 |
| 116 | 31.0 |
| 122 | 17.0 |

### Affinity for H1Histaminergic and σ Receptors

Experimental conditions for tests are given in Table 14, and results of tests for representative compounds are presented in Table 15.

TABLE 14
Experimental conditions tor testing the affinity for H1
histaminergic and σ receptors

| | σ | H1 |
|---|---|---|
| Biological | rat cerebral cortex | human recombinant |
| material | | (HEK-293 cells) |
| Radioligand | [³H]DTG | [³H]pyrilamine |
| Concentration | 8 nM | 1 nM |
| Kd | 29 nM | 1.7 nM |
| Non-specific | haloperidol (10 μM) | pyrilamine (1 μM) |
| binding | | |
| Incubation | 120 min, 22° C. | 60 min, 22° C. |
| Methodology | Shirayama et al. (1993). | Smit et al. (1996), . |
| | Eur. J. Pharmacol., | Brit. J Pharmacol., |
| | 237: 117-126 | 117: 1071-1080. |

TABLE 15
Results of σ and H1 receptors affinity tests for
representative compounds of the invention

| Compound No. | σ [%] | H1 [%] |
|---|---|---|
| 11 | 74 | 37 |
| 20 | 83 | 36 |
| 21 | 60 | 57 |
| 23 | 61 | 60 |
| 25 | 70 | 75 |
| 26 | 80 | 44 |
| 28 | 70 | 63 |
| 29 | 60 | 59 |
| 34 | 50 | 56 |
| 39 | 54 | |
| 46 | 15 | 6 |
| 47 | 44 | −3 |
| 48 | 18 | 21 |
| 49 | 50 | |
| 50 | 59 | 34 |
| 55 | 37 | 0 |
| 75 | 75 | 57 |
| 77 | 48 | 54 |
| 78 | 70 | 65 |
| 79 | 67 | 72 |
| 80 | 81 | |
| 82 | 63 | 61 |
| 84 | 59 | 79 |
| 86 | 65 | 70 |
| 89 | 24 | 3 |
| 92 | 15 | 6 |
| 93 | 35 | 34 |
| 94 | 71 | |
| 107 | 47 | 30 |
| 108 | 45 | 23 |
| 109 | 39 | 65 |
| 110 | 56 | 52 |
| 111 | 81 | 38 |
| 115 | 69 | 72 |
| 116 | 56 | |
| 122 | 81 | |
| 124 | 82 | |
| 127 | | 54 |
| 130 | 94 | 51 |
| 131 | 92 | 51 |
| 132 | 80 | 33 |
| 133 | 93 | 58 |
| 134 | 88 | 45 |
| 135 | 94 | 79 |
| 143 | | 36 |
| 181 | 75 | 27 |
| 183 | 94 | 42 |
| 186 | 84 | 17 |
| 196 | | 84 |
| 197 | | 76 |
| 198 | 86 | 81 |
| 199 | 80 | 58 |
| 200 | | |
| 201 | | 64 |
| 203 | | 67 |
| 204 | 90 | |
| 205 | 92 | 50 |
| 207 | | 65 |
| 214 | 84 | 52 |
| 215 | 81 | 50 |
| 216 | 88 | 61 |
| 217 | 86 | 46 |
| 218 | | 77 |
| 221 | 83 | 66 |
| 222 | 95 | 79 |
| 223 | 89 | 52 |
| 224 | 91 | 60 |
| 225 | 96 | 57 |
| 226 | 82 | 73 |
| 227 | 59 | 69 |
| 229 | | 75 |
| 230 | 90 | 71 |
| 231 | 87 | 54 |
| 234 | | 75 |
| 237 | 88 | 66 |
| 238 | 95 | 65 |
| 239 | 97 | |
| 240 | 92 | 55 |
| 241 | 88 | 69 |
| 293 | | 38 |
| 298 | | 40 |
| 300 | | 28 |
| 303 | | 68 |
| 305 | | 54 |
| 306 | | 57 |
| 308 | 91 | 37 |
| 312 | 92 | 35 |
| 317 | | 47 |

Ability to Block hERG Potassium Channel

Ability to block hERG potassium channels was determined using the electrophysiological method and cloned hERG potassium channels (KCNH2 gene, expressed in CHO cells) as biological material. The effects were evaluated using IonWorks™ Quattro system (MDS-AT).

hERG current was elicited using a pulse pattern with fixed amplitudes (conditioning pre-pulse: −80 mV for 25 ms; test pulse: +40 mV for 80 ms) from a holding potential of 0 mV. hERG current was measured as a difference between the peak current at 1 ms after the test step to +40 mV and the steady-state current at the end of the step to +40 mV.

### Data Analysis

Data acquisition and analyses was performed using the IonWorks Quattro™ system operation software (version 2.0.2; Molecular Devices Corporation, Union City, Calif.). Data were corrected for leak current.

The hERG block was calculated as:

% Block=(1−ITA/IControl)×100%,

where IControl and ITA were the currents elicited by the test pulse in control and in the presence of a test article, respectively.

Concentration-response data for the blocks were fit to an equation of the following form:

% Block=% VC+{(% PC−% VC)−(% PC−% VC)/[1+([Test]/IC50)N]},

where [Test] is the concentration of test article, IC50 was the concentration of the test article producing half-maximal inhibition, N was the Hill coefficient, % VC was the percentage of the current run-down (the mean current inhibition at the vehicle control), % PC was the mean inhibition of the current with the positive control (1 μM E-4031) and % Block was the percentage of ion channel current inhibited at each concentration of a test article. Nonlinear least squares fits were solved with the XLfit add-in for Excel 2003 (Microsoft, Redmond, Wash.).

Results of tests for representative compounds are presented in Table 16.

TABLE 16
Results of hERG potassium channels affinity tests
for representative compounds of the invention

| Compound No. | hERG [%] |
|---|---|
| 21 | 3.3 |
| 25 | 2.9 |
| 26 | 6.1 |
| 34 | −1.1 |
| 94 | 4.8 |

Results of in vitro tests as presented above show that compounds of the invention display high affinity for D2, D3, 5-HT1A, 5-HT2A, 5-HT6, 5-HT7, as well as for adrenergic receptors and for serotonin transporter. This confirms their potential usefulness in the treatment of diseases connected with disturbances in dopaminergic, serotoninergic and noradrenergic transmission, e.g. psychoses, depression as well as anxiety disorders etc. It should be stressed that some of the compounds possess simultaneously high affinity for 5-HT6 and 5-HT7 as well as for D2, and 5-HT2A receptors, what particularly distinguishes them from drugs currently used in therapy. Such a pharmacological profile suggests possible efficacy in the treatment of psychoses as well as antidepressant and procognitive activity. At the same time compounds of the invention possess weak affinity for hERG potassium channel and M3 muscarinic receptor, and in straight majority low affinity for H1 and 5-HT2C receptors. This may potentially contribute to lack of side effects such as excessive appetite or metabolic disorders, which may be caused by drugs currently used in therapy of the above-mentioned diseases.

### EXAMPLE 4

### In Vitro Pharmacology: Cellular Functional Assays

Conditions and methodology (by reference to the literature) of cellular functional assays are given in Table 17 and the tests results for representative compounds of the invention are presented in Tables 18, 19, 20, 21, 22 and 23.

The results are expressed as a percent of control specific agonist response ((measured specific response/control specific agonist response)×100) obtained in the presence of the test compounds.

The EC50 values (concentration producing a half-maximal specific response) and IC50 values (concentration causing a half-maximal inhibition of the control specific agonist response) were determined by non-linear regression analysis of the concentration-response curves generated with mean replicate values using Hill equation curve fitting (Y=D+[(A−D)/(1+(C/C50)nH)], where Y=specific response, D=minimum specific response, A=maximum specific response, C=compound concentration, and C50=EC50 or IC50, and nH=slope factor). This analysis was performed using a software developed at Cerep (Hill software) and validated by comparison with data generated by the commercial software SigmaPlot® 4.0 for Windows® (© 1997 by SPSS Inc.).

For the antagonists, the apparent dissociation constants (Kb) were calculated using the modified Cheng Prusoff equation (Kb=IC50/(1+(A/EC50A)), where A=concentration of reference agonist in the assay, and EC50A=EC50 value of the reference agonist).

| | | | | Reaction | | |
|---|---|---|---|---|---|---|
| Assay | Origin | Stimulus | Incubation | product | method of detection | Literature |
| D2S (h) | human recombinant, | none (3 μM dopamine | 28° C. | impedance | cellular dielectric | Payne et al. (2002), J. |
| (agonism) | (HEK-293 cells) | for control) | | | spectroscopy | Neurochem., 82: 1106-1117 |
| D2S (h) | human recombinant, | dopamine (30 nM) | 28° C. | impedance | cellular dielectric | Payne et al. (2002), J. |
| (antagonism) | (HEK-293 cells) | | | | spectroscopy | Neurochem., 82: 1106-1117 |
| D3 (h) | human recombinant, | none (0.3 μM | 10 min. | cAMP | HTRF (Homogenous | Missale et al. (1998), Physiol. Rev., |
| (agonism) | (CHO cells) | dopamine for control) | 37° C. | | Time Resolved | 78: 189-225 |
| | | | | | Fluorescence) | |
| D3 (h) | human recombinant, | dopamina (10 nM) | 10 min. | cAMP | HTRF | Missale et al. (1998), Physiol. Rev., |
| (antagonism) | (CHO cells) | | 37° C. | | | 78: 189-225 |
| D4.4 (h) | human recombinant | none (300 nM | 10 min | cAMP | HTRF | Missale et al. (1998), Physiol. Rev., |
| (agonism) | (CHO cells) | dopamine for control) | 37° C. | | | 78: 189-225 |
| D4.4 (h) | human recombinant | dopamine | 10 min | cAMP | HTRF | Missale et al. (1998), Physiol. Rev., |
| (antagonistm) | (CHO cells) | (100 nM) | 37° C. | | | 78: 189-225 |
| 5-HT1A (h) | human recombinant, | none (100 nM 8- | 30 min. | cAMP | HTRF | Newman-tancredi et al. (2001), |
| (agonism) | (CHO cells) | OHDPAT for control) | 22° C. | | | Brit. J. Pharmacol., 132: 518-524 |
| 5-HT1A (h) | human recombinant, | 8-OH-DPAT (10 nM) | 30 min. | cAMP | HTRF | Newman-tancredi et al. (2001), |
| (antagonism) | (CHO cells) | | 22° C. | | | Brit. J. Pharmacol., 132: 518-524 |
| 5-HT2A (h) | human recombinant, | none (10 μM serotonin | 30 min. | IP1 | HTRF | Porter et al. (1999), Brit. J. |
| (agonism) | (HEK-293 cells) | for control) | 37° C. | | | Pharmacol., 128: 13-20 |
| 5-HT2A (h) | human recombinant, | serotonin (100 nM) | 30 min. | IP1 | HTRF | Porter et al. (1999), Brit. J. |
| (antagonism) | (HEK-293 cells) | | 37° C. | | | Pharmacol., 128: 13-20 |
| 5-HT6 (h) | human recombinant, | none (10 μM serotonin | 45 min. | cAMP | HTRF | Kohen et al. (1996), J. |
| (agonism) | (CHO cells) | for control) | 37° C. | | | Neurochem., 66: 47-56 |
| 5-HT6 (h) | human recombinant, | serotonin (100 nM) | 45 min. | cAMP | HTRF | Kohen et al. (1996), J. |
| (antagonism) | (CHO cells) | | 37° C. | | | Neurochem., 66: 47-56 |
| 5-HT7 (h) | human recombinant, | none (10 μM serotonin | 45 min. | cAMP | HTRF | Adham et al. (1998), J. Pharmacol. |
| (agonism) | (CHO cells) | for control) | 37° C. | | | Exp. Ther., 287: 508-514 |
| 5-HT7 (h) | human recombinant, | serotonin (300 nM) | 45 min. | cAMP | HTRF | Adham et al. (1998), J. Pharmacol. |
| (antagonism) | (CHO cells) | | 37° C. | | | Exp. Ther., 287: 508-514 |
| 5-HT2C (h) | human recombinant | none (1 μM serotonin | 30 min | IP1 | HTRF | Porter et al. (1999), Brit. J. |
| (agonism) | (HEK-293 cells) | for control) | 37° C. | | | Pharmacol., 128: 13-20 |
| 5-HT2C (h) | human recombinant | Serotonin (10 nM) | 30 min | IP1 | HTRF | Porter et al. (1999), Brit. J. |
| (antagonism) | (HEK-293 cells) | | 37° C. | | | Pharmacol., 128: 13-20 |

TABLE 18
Results of cellular functional assays for D2 and D3 dopaminergic
receptors for representative compounds of the invention

| Compound | D2 ag | D2 antag | D3 ag | D3 antag |
|---|---|---|---|---|
| No. | [%] | [%] | [%] | [%] |
| 20 | 4 | 32 | | |
| 21 | 6 | 98 | −8 | 83 |
| 23 | 1 | 87 | −10 | 68 |
| 25 | 3 | 89 | 0 | 82 |
| 26 | 3 | 68 | | |
| 28 | 1 | 87 | −8 | 74 |
| 39 | | | 66 | 16 |
| 49 | 57 | 97 | 75 | 12 |
| 50 | 56 | 92 | 85 | 0 |
| 55 | | | 82 | −33 |
| 78 | | | 89 | −10 |
| 80 | 33 | 78 | 91 | −5 |
| 94 | 45 | 99 | 51 | 38 |
| 111 | 36 | 89 | | |
| 122 | 33 | 62 | 38 | 56 |
| 124 | 33 | 44 | 11 | 43 |
| 130 | 6 | 34 | 5 | 61 |
| 131 | 5 | 20 | −13 | 55 |
| 134 | 6 | 47 | | |
| 183 | 8 | 53 | | |
| 196 | 0 | 95 | −16 | 77 |
| 197 | 1 | 82 | | |
| 198 | 4 | 83 | −17 | 97 |
| 199 | 4 | 46 | | |
| 200 | 0 | 96 | −1 | 93 |
| 202 | 0 | 95 | −5 | 81 |
| 203 | 0 | 80 | 11 | 65 |
| 204 | 1 | 86 | −8 | 109 |
| 205 | 3 | 61 | 7 | 95 |
| 206 | 1 | 96 | 3 | 77 |
| 214 | 3 | 89 | | 117 |
| 215 | 6 | 91 | | |
| 216 | 0 | 86 | | |
| 217 | 10 | 79 | | |
| 221 | 14 | 97 | −3 | 100 |
| 222 | 6 | 65 | | |
| 223 | | | | |
| 224 | 5 | 57 | | |
| 225 | 7 | 52 | | |
| 226 | 0 | 87 | −26 | 107 |
| 227 | 11 | 99 | −6 | 105 |
| 228 | −1 | 94 | −15 | 98 |
| 230 | 3 | 94 | 0 | 89 |
| 231 | 13 | 49 | −6 | 54 |
| 234 | 2 | 91 | −14 | 89 |
| 239 | 2 | 56 | | |
| 240 | 3 | 64 | | |

TABLE 19
Results of cellular functional assays for D4
dopaminergic receptors for
representative compounds of the invention

| Compound | D4 ag | D4 antag |
|---|---|---|
| No. | [%] | [%] |
| 20 | 17 | 79 |
| 26 | −4 | 88 |
| 50 | 62 | 32 |
| 80 | 6 | 80 |
| 94 | 60 | 9 |
| 221 | 10 | 73 |
| 222 | 6 | 58 |
| 223 | 4 | 22 |
| 224 | −7 | 84 |
| 225 | −3 | 35 |

TABLE 20
Results of cellular functional assays for 5-HT1A, 5-HT2A, 5-HT6 and 5-HT7
serotoninergic receptors for representative compounds of the invention

| Compound | 5-HT1A | 5-HT1A | 5-HT2A | 5-HT2A | 5-HT6 | 5-HT6 | 5-HT7 | 5-HT7 |
|---|---|---|---|---|---|---|---|---|
| No. | ag [%] | antag [%] | ag [%] | antag [%] | ag [%] | antag [%] | ag [%] | antag [%] |
| 20 | | | 0 | 88 | 1 | 68 | −2 | 82 |
| 21 | | | 2 | 85 | 1 | 55 | −1 | 87 |
| 23 | | | −2 | 81 | −1 | 52 | −2 | 36 |
| 25 | | | −2 | 99 | 0 | 91 | −2 | 99 |
| 26 | | | −1 | 96 | 0 | 101 | −3 | 95 |
| 28 | | | −2 | 84 | −1 | 91 | −3 | 68 |
| 34 | | | −1 | 59 | | | −1 | 99 |
| 39 | 34 | 70 | 3 | 65 | −1 | 6 | 1 | 40 |
| 49 | 39 | 97 | | | | | 26 | 70 |
| 50 | 48 | 92 | −2 | 63 | | | 21 | 78 |
| 55 | 72 | 18 | −1 | | | | 5 | 48 |
| 78 | 54 | 76 | 51 | −1 | 54 | 19 | | |
| 80 | 42 | 66 | 34 | 23 | 9 | 54 | 3 | 38 |
| 92 | 47 | 89 | | | | | 18 | 69 |
| 93 | 37 | 76 | | | | | 13 | 76 |
| 94 | 41 | 90 | 7 | 48 | | | 19 | 68 |
| 111 | 48 | 89 | 5 | 58 | | | 24 | 55 |
| 122 | 35 | 83 | | | 0 | 78 | 21 | 39 |
| 124 | 16 | 77 | | | 7 | 72 | 10 | 1 |
| 130 | | | 0 | 84 | 4 | 65 | 0 | 59 |
| 131 | | | −2 | 69 | 3 | 53 | 1 | 45 |
| 134 | | | −3 | 92 | 4 | 62 | 1 | 64 |
| 181 | 66 | 100 | | | | | 0 | 33 |
| 183 | 71 | 103 | 1 | 72 | | | 2 | 82 |
| 196 | | | 0 | 98 | −2 | 75 | 0 | 97 |
| 197 | | | 1 | 96 | −1 | 59 | 0 | 94 |
| 198 | | | −3 | 98 | −1 | 37 | 1 | 19 |
| 199 | | | −2 | 96 | −3 | −1 | 1 | 42 |
| 200 | | | 0 | 56 | −2 | 53 | −1 | 40 |
| 202 | | | 0 | 90 | 1 | 67 | −3 | 73 |
| 203 | | | −1 | 90 | −2 | 62 | 0 | 77 |
| 204 | | | −1 | 94 | −1 | 49 | | |
| 205 | | | −3 | 87 | 1 | 20 | −1 | 38 |
| 206 | | | −1 | 96 | −2 | 69 | −1 | 97 |
| 214 | | | −2 | 98 | −4 | 76 | −1 | 96 |
| 215 | | | −2 | 100 | −2 | 50 | 0 | 90 |
| 216 | | | −2 | 100 | −5 | 80 | −5 | 90 |
| 217 | | | −2 | 101 | −6 | 85 | 5 | 74 |
| 221 | | | −2 | 100 | −4 | 79 | 2 | 80 |
| 222 | | | −1 | 96 | −1 | 70 | −1 | 95 |
| 223 | | | 0 | 96 | −1 | 66 | 0 | 85 |
| 224 | | | −2 | 97 | −1 | 87 | −1 | 98 |
| 225 | | | −1 | 98 | 1 | 89 | 0 | 84 |
| 226 | | | −1 | 96 | −5 | 48 | −2 | 10 |
| 227 | | | −2 | 103 | −4 | 66 | −2 | 30 |
| 228 | | | 1 | 82 | −2 | 50 | 0 | 27 |
| 230 | | | −2 | 101 | −1 | 51 | | |
| 231 | | | 0 | 94 | | _{—} | | |
| 234 | | | 1 | 98 | −1 | 51 | 0 | 70 |
| 239 | | | −2 | 73 | 2 | 82 | 1 | 63 |
| 240 | | | −1 | 89 | 4 | 61 | 0 | 63 |

TABLE 21
Results of cellular functional assays for
5-HT2C serotoninergic receptors for
representative compounds of the invention

| Compound | 5-HT2C ag | 5-HT2C antag |
|---|---|---|
| No. | [%] | [%] |
| 94 | 92 | −57 |
| 122 | 78 | −10 |
| 181 | 46 | 6 |
| 183 | 50 | 12 |
| 203 | 1 | 33 |
| 228 | 0 | 30 |
| 230 | −2 | 5 |

TABLE 22
Cellular functional profile for the representative
compounds of the invention

| | D2 | D3 | 5-HT2A | 5-HT6 | 5-HT7 |
|---|---|---|---|---|---|
| | antag | antag | antag | antag | antag |
| Compound | Kb | Kb | Kb | Kb | Kb |
| No. | [nM] | [nM] | [nM] | [nM] | [nM] |
| 21 | 1.6 | 7.3 | 8.3 | | 1.7 |
| 25 | 2.3 | 8.0 | 4.7 | 10.0 | 1.4 |
| 34 | | | | | 0.077 |
| 196 | 10.0 | 71.0 | 8.3 | 38.0 | 0.57 |

5-HT reuptake was tested according to Perovic, S. and Muller, W.E.G. (1995) Arzneim-Forsch. Drug Res., 45: 1145-1148 by measuring [³H]5-HT incorporation into rat brain synaptosomes. Assay conditions are as follows:

### Tracer: [³H]5-HT (0.2 μCi/ml)

Incubation: 15 min/37° C.
Detection method: Scintillation counting
Reference: imipramine (IC₅₀:30 nM)

TABLE 23

| Compound | SERT |
|---|---|
| No. | IC50 [nM] |
| 80 | 2.1 |
| 84 | 64.0 |
| 86 | 17.0 |

Compounds of invention displayed significant antagonistic properties at 5-HT6 and/or 5-HT7 receptors which was either isolated or combined with some other beneficial properties like blockade of dopaminergic D2 and serotonin 5-HT2A receptors and/or 5-HT1A receptor partial agonism. Some of the compounds of invention possessed also ability to inhibit serotonin uptake. Selected compounds of invention, possessing significant affinity for 5-HT2C receptor were found to either be weak antagonists or display agonistic profile. Those properties, taken together with their low affinity for muscarinic receptors or hERG channels, indicate potential usefulness of the compounds of invention in the treatment of numerous CNS disorders, especially psychotic states, as well as mood disorders and cognitive deficits.

### EXAMPLE 5

### Behavioral Tests in Mice

### Antipsychotic Activity in Mice

Potential antipsychotic activity was tested for the representative compounds in mouse model of psychosis, involving the induction of locomotor hyperactivity by administering psychotomimetic substance—dizocilpine. The ability of a test compound to remove this effect is a measure of potential antipsychotic activity.

### Animals

Male CD-1 mice were group-housed for 2-3 day period in polycarbonate Makrolon type 3 cages (dimensions 26.5×15×42 cm) in an environmentally controlled, experimental room (ambient temperature 22-20° C.; relative humidity 50-60%; 12:12 light:dark cycle, lights on at 8:00), in groups of 15. Standard laboratory food (Ssniff M-Z) and filtered water were freely available. On the day before experiments the equipment produced “white noise” was turned on for 30 minutes and mice were weighted exact to 1 g. Animals were assigned randomly to treatment groups. All the experiments were performed by two observers unaware of the treatment applied between 9:00 and 14:00 on separate groups of animals. Mice were used only once and were killed immediately after the experiment.

### Dizocilpine-Induced Locomotor Hyperactivity

The locomotor activity was recorded with an Opto M3 multi-channel activity monitor (MuttiDevice Software v.1.3, Columbus Instruments). The mice were individually placed in plastic cages (22×12×13 cm) for 30 minutes habituation period, and then the crossings of each channel (ambulation) were counted during 1 h with data recording every 5 minutes. The cages were cleaned up with 70% ethanol after examining each mouse. Drugs were administered to 10 mice per treatment group. Test compounds were given 30 minutes before the experiment. Dizocilpine was administered 30 minutes before the test.

### Test Compounds

Test compounds were prepared as a suspension in 1% aqueous solution of Tween 80, and dizocilpine was dissolved in distilled water immediately before administration. An injection volume of 10 ml/kg was used and all compounds were administered intraperitoneally (i.p.).

TABLE 24
Results of behavioural test in mice for the representative
compounds of the invention—reversal of dizocilpine
(MK-801)-induced hyperlocomotion in mice

| Compound No. | MED* [mg/kg] |
|---|---|
| 21 | 10 |
| 25 | 5 |
| 196 | 2.5 |

*minimum effective dose

Dizocilpine (MK-801) is widely recognized as a useful pharmacological tool for modeling of psychotic states in animals by causing glutamatergic dysregulation, similar to that occurring in humans. Ability of the compounds of invention to reverse the dizocilpine-induced hyperlocomotion proves their antipsychotic-like activity in animals and additionally confirms their therapeutic potential in treatment of psychotic states in humans.

1. Compound of the general formula (I) 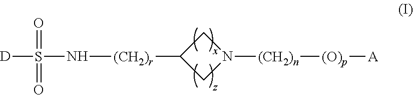
wherein
A represents naphthyl or 9- or 10-membered bicyclic group, linked to —(O)ₚ—(CH₂)ₙ through one of its aromatic carbon atoms, consisting of benzene ring fused with:
5-membered heteroaromatic ring having 1 heteroatom selected from N and S or 2 heteroatoms independently selected from N, O, and S, wherein such a bicyclic group may be unsubstituted or substituted with halogen atom; or
5- or 6-membered heterocyclic non-aromatic ring having 1 or 2 heteroatoms independently selected from N and O, wherein heterocyclic ring may be unsubstituted or substituted with ═O or one or more C₁-C₃-alkyls;
D represents a moiety selected from the group consisting of:
phenyl unsubstituted or substituted with one or more substituents independently selected from the group consisting of C₁-C₄-alkyl, C₁-C₃-alkyloxy, halogeno-C₁-C₃-alkyl, halogen atom, halogeno-C₁-C₃-alkyloxy-, —CN, —OH, and phenyl;
naphthyl unsubstituted or substituted with one or more substituents independently selected from the group consisting of C₁-C₄-alkyl, C₁-C₃-alkyloxy and halogen atom;
5-membered aromatic heterocyclic group having 1 or 2 heteroatoms independently selected from N, O, and S, unsubstituted or substituted with one or more substituents independently selected from the group consisting of C₁-C₄-alkyl, halogen atom, and 5-membered heteroaromatic ring having or 2 heteroatoms independently selected from N and O, linked to sulphonamide group through one of its aromatic carbon atoms; and
bicyclic group consisting of a ring selected from benzene and pyridine, fused with 5-membered aromatic or non-aromatic heterocyclic ring, having 1 or 2 heteroatoms independently selected from N, O, and S, unsubstituted or substituted with one or more substitutents independently selected from the group consisting of C₁-C₄-alkyl, halogen atom, and ═O, linked to sulphonamide moiety through one of its aromatic carbon atoms;
r represents 0 or 1;
x and z represent independently 1 or 2;
n represents 3 and p represents 0, or n represents 2 and p represents 1;
and enantiomers, pharmaceutically acceptable salts and solvates thereof.

2. The compound according to claim 1, wherein D represents the moiety selected from the group consisting of:
phenyl unsubstituted or substituted with one or more substituents independently selected from the group consisting of C₁-C₄-alkyl, C₁-C₃-alkyloxy, halogeno-C₁-C₃-alkyl, halogen atom, —CN, —OH, and phenyl;
naphthyl unsubstituted or substituted with one or more substituents independently selected from the group consisting of C₁-C₄-alkyl and halogen atom;
5-membered aromatic heterocyclic group having 1 or 2 heteroatoms independently selected from N, O, S, unsubstituted or substituted with one or more substituents independently selected from the group consisting of C₁-C₄-alkyl, halogen atom, 5-membered heteroaromatic ring having 1 or 2 heteroatoms independently selected from N, O; linked to sulphonamide group through one of its aromatic carbon atoms; and
bicyclic group consisting of a ring selected from benzene and pyridine, fused with 5-membered aromatic or non-aromatic heterocyclic ring, having 1 or 2 heteroatoms independently selected from N, O, and S, unsubstituted or substituted with one or more substitutents independently selected from the group consisting of C₁-C₄-alkyl, halogen atom, and ═O, linked to sulphonamide moiety through one of its aromatic carbon atoms.

3. The compound according to claim 2, wherein A represents naphthyl.

4. The compound according to claim 1, wherein A represents 9-membered bicyclic group consisting of benzene ring fused with 5-membered monoheteroaromatic ring having atom N.

5. The compound according to claim 1, wherein A represents 9-membered bicyclic group consisting of benzene ring fused with 5-membered heteroaromatic ring having 2 heteroatoms independently selected from N, O, and S.

6. The compound according to claim 1, wherein A represents 10-membered bicyclic group consisting of benzene ring fused with 6-membered heterocyclic ring having 1 or 2 heteroatoms independently selected from N and O.

7. The compound according to claim 1, wherein A represents 9-membered bicyclic group, consisting of benzene ring fused with non-aromatic 5-membered heterocyclic ring having 1 or 2 heteroatoms independently selected from N and O, wherein heterocyclic ring is substituted with ═O or one or more C₁-C₃-alkyls.

8. The compound according to claim 1, wherein D represents phenyl unsubstituted or substituted with one or more substituents independently selected from the group consisting of C₁-C₄-alkyl, C₁-C₃-alkyloxy, halogeno-C₁-C₃-alkyl, halogen atom, halogeno-C₁-C₃-alkyloxy-, —CN, —OH, and phenyl.

9. The compound according to claim 1, wherein D represents naphthyl unsubstituted or substituted with one or more substituents independently selected from the group consisting of C₁-C₄-alkyl, C₁-C₃-alkyloxy and halogen atom.

10. The compound according to claim 1, wherein D represents bicyclic group consisting of benzene ring fused with 5-membered aromatic or non-aromatic heterocyclic ring, having 1 or 2 heteroatoms independently selected from N, O, and S, unsubstituted or substituted with one or more substitutents independently selected from the group consisting of C₁-C₄-alkyl, halogen atom, and ═O.

11. The compound according to claim 1 wherein n is 3 and p is 0.

12. The compound according to claim 1 wherein n is 2 and p is 1.

13. The compound according to claim 1 wherein x and z are both 2.

14. The compound according to claim 1 wherein x is 2 and z is 1.

15. The compound according to claim 1 wherein x and z are both 1.

16. The compound according to claim 1 wherein r is 0.

17. The compound according to claim 1 wherein r is 1.

18. The compound according to claim 1 selected from the group consisting of the following:
N-[1-[3-(6-fluoro-1,2-benzoxazol-3-yl)propyl]azetidin-3-yl]benzenesulphonamide,
3-fluoro-N-[1-[3-(6-fluoro-1,2-benzoxazol-3-yl)propyl]azetidin-3-yl]benzene-sulphonamide,
4-fluoro-N-[1-[3-(6-fluoro-1,2-benzoxazol-3-yl)propyl]azetidin-3-yl]benzene-sulphonamide,
3-chloro-N-[1-[3-(6-fluoro-1,2-benzoxazol-3-yl)propyl]azetidin-3-yl]benzene-sulphonamide,
N-[1-[3-(6-fluoro-1,2-benzoxazol-3-yl)propyl]azetidin-3-yl]-3-methylbenzene-sulphonamide,
N-[1-(3-(6-fluoro-1,2-benzoxazol-3-yl)propyl)pyrrolidin-3-yl]benzenesulphonamide,
3-fluoro-N-[1-[3-(6-fluoro-1,2-benzoxazol-3-yl)propyl]pyrrolidin-3-yl]benzene-sulphonamide,
4-fluoro-N-[1-[3-(6-fluoro-1,2-benzoxazol-3-yl)propyl]pyrrolidin-3-yl]benzene-sulphonamide,
3-chloro-N-[1-[3-(6-fluoro-1,2-benzoxazol-3-yl)propyl]pyrrolidin-3-yl]benzene-sulphonamide,
4-bromo-N-[1-[3-(6-fluoro-1,2-benzoxazol-3-yl)propyl]pyrrolidin-3-yl]benzene-sulphonamide,
4-chloro-3-fluoro-N-[1-[(3-(6-fluoro-1,2-benzoxazol-3-yl)propyl]pyrrolidin-3-yl]benzenesulphonamide,
N-[1-[3-(6-fluoro-1,2-benzoxazol-3-yl)propyl]pyrrolidin-3-yl]-3-methylbenzene-sulphonamide,
N-[1-[3-(6-fluoro-1,2-benzoxazol-3-yl)propyl]pyrrolidin-3-yl]-4-propylbenzene-sulphonamide,
4-tert-butyl-N-[1-[3-(6-fluoro-1,2-benzoxazol-3-yl)propyl]pyrrolidin-3-yl)benzenesulphonamide,
N-[1-[3-(6-fluoro-1,2-benzoxazol-3-yl)propyl]pyrrolidin-3-yl]-3-(trifluoromethyl)-benzenesulphonamide,
N-[1-(3-(6-fluoro-1,2-benzoxazol-3-yl)propyl]pyrrolidin-3-yl]-4-(trifluoromethyl)-benzenesulphonamide,
N-[1-[3-(6-fluoro-1,2-benzoxazol-3-yl)propyl]pyrrolidin-3-yl]-3-methoxybenzenesulphonamide,
N-[1-[3-(6-fluoro-1,2-benzoxazol-3-yl)propyl]pyrrolidin-3-yl]-3-hydroxy-benzenesulphonamide,
3-cyano-N-[1-[3-(6-fluoro-1,2-benzoxazol-3-yl)propyl]pyrrolidin-3-yl]benzene-sulphonamide,
N-[1-[3-(6-fluoro-1,2-benzoxazol-3-yl)propyl]pyrrolidin-3-yl]naphthalene-1-sulphonamide,
N-[1-[3-(6-fluoro-1,2-benzoxazol-3-yl)propyl]pyrrolidin-3-yl]naphthalene-2-sulphonamide,
5-chloro-N-[1-[3-(6-fluoro-1,2-benzoxazol-3-yl)propyl]pyrrolidin-3-yl]thiophene-2-sulphonamide,
6-chloro-N-[1-[3-(6-fluoro-1,2-benzoxazol-3-yl)propyl]pyrrolidin-3-yl]naphthalene-2-sulphonamide,
N-[1-[3-(6-fluoro-1,2-benzoxazol-3-yl)propyl]pyrrolidin-3-yl]-2,3-dihydrobenzofurano-6-sulphonamide,
N-[1-[3-(6-fluoro-1,2-benzoxazol-3-yl)propyl]pyrrolidin-3-yl]benzothiophene-2-sulphonamide,
N-[1-[3-(6-fluoro-1,2-benzoxazol-3-yl)propyl]pyrrolidin-3-yl]benzothiophene-3-sulphonamide,
6-chloro-N-[1-[3-(6-fluoro-1,2-benzoxazol-3-yl)propyl]pyrrolidin-3-yl]benzothiophene-2-sulphonamide,
5-fluoro-N-[1-[3-(6-fluoro-1,2-benzoxazol-3-yl)propyl]pyrrolidin-3-yl]-3-methyl-benzothiophene-2-sulphonamide,
5-chloro-N-[1-[3-(6-fluoro-1,2-benzoxazol-3-yl)propyl]pyrrolidin-3-yl]-3-methyl-benzothiophene-2-sulphonamide,
N-[1-[3-(6-fluoro-1,2-benzoxazol-3-yl)propyl]pyrrolidin-3-yl]imidazo[1,2-a]pyridine-3-sulphonamide,
N-[1-[3-(6-fluoro-1,2-benzoxazol-3-yl)propyl]pyrrolidin-3-yl]-1,3-benzothiazole-4-sulphonamide,
N-[1-[3-(6-fluoro-1,2-benzoxazol-3-yl)propyl]-4-piperidine]benzenesulphonamide,
N-[1-[3-(6-fluoro-1,2-benzoxazol-3-yl)propyl]-4-piperidine]-3-methylbenzene-sulphonamide,
N-[1-[3-(6-fluoro-1,2-benzoxazol-3-yl)propyl]-4-piperidine]naphthalene-1-sulphonamide,
N-[1-[3-(6-fluoro-1,2-benzoxazol-3-yl)propyl]-4-piperidine]naphthalene-2-sulphonamide,
N-[[1-[3-(6-fluoro-1,2-benzoxazol-3-yl)propyl]-4-piperidine]methyl]-3-methyl-benzenesulphonamide,
N-[[1-[3-(6-fluoro-1,2-benzoxazol-3-yl)propyl]-4-piperidine]methyl]naphthalene-1-sulphonamide,
N-[[1-[3-(6-fluoro-1,2-benzoxazol-3-yl)propyl]-4-piperidine]methyl]naphthalene-2-sulphonamide,
N-[1-[2-(1,2-benzothiazol-3-yloxy)ethyl]pyrrolidin-3-yl]naphthalene-2-sulphonamide,
N-[1-[2-(1,2-benzothiazol-3-yloxy)ethyl]-4-piperidine]naphthalene-2-sulphonamide,
N-[[1-[2-(1,2-benzothiazol-3-yloxy)ethyl]azetidin-3-yl]methyl]-3-hydroxy-benzene-sulphonamide,
N-[[1-[2-(1,2-benzothiazol-3-yloxy)ethyl]azetidin-3-yl]methyl]naphthalene-2-sulphonamide,
N-[[1-[2-(1,2-benzothiazol-3-yloxy)ethyl]pyrrolidin-3-yl]methyl]-3-hydroxy-benzene-sulphonamide,
N-[[1-[2-(1,2-benzothiazol-3-yloxy)ethyl]pyrrolidin-3-yl]methyl]naphthalene-2-sulphonamide,
N-[1-[2-(1H-indol-4-yloxy)ethyl]azetidin-3-yl]benzenesulphonamide,
4-fluoro-N-[1-[2-(1H-indol-4-yloxy)ethyl]azetidin-3-yl]benzenesulphonamide,
3-chloro-N-[1-[2-(1H-indol-4-yloxy)ethyl]azetidin-3-yl]benzenesulphonamide,
N-[1-[2-(1H-indol-4-yloxy)ethyl]azetidin-3-yl]-3-methyl-benzenesulphonamide,
N-[1-[2-(1H-indol-4-yloxy)ethyl]azetidin-3-yl]naphthalene-1-sulphonamide,
N-[1-[2-(1H-indol-4-yloxy)ethyl]azetidin-3-yl]naphthalene-2-sulphonamide,
N-[1-[2-(1H-indol-4-yloxy)ethyl]pyrrolidin-3-yl]benzenesulphonamide,
N-[1-[2-(1H-indol-4-yloxy)ethyl]pyrrolidin-3-yl]-3-methylbenzenesulphonamide,
N-[1-[2-(1H-indol-4-yloxy)ethyl]pyrrolidin-3-yl]naphthalene-1-sulphonamide,
N-[1-[2-(1H-indol-4-yloxy)ethyl]pyrrolidin-3-yl]naphthalene-2-sulphonamide,
N-[1-[2-(1H-indol-4-yloxy)ethyl]-4-piperidine]benzenesulphonamide,
N-[1-[2-(1H-indol-4-yloxy)ethyl]-4-piperidine]-3-methylbenzenesulphonamide,
4-tert-butyl-N-[1-[2-(1H-indol-4-yloxy)ethyl]-4-piperidine]benzenesulphonamide,
N-[1-[2-(1H-indol-4-yloxy)ethyl]-4-piperidine]-4-(trifluoromethyl)benzenesulphonamide,
4-cyano-N-[1-[2-(1H-indol-4-yloxy)ethyl]-4-piperidine]benzenesulphonamide,
N-[1-[2-(1H-indol-4-yloxy)ethyl]-4-piperidine]naphthalene-1-sulphonamide,
N-[1-[2-(1H-indol-4-yloxy)ethyl]-4-piperidine]naphthalene-2-sulphonamide,
5-chloro-N-[1-[2-(1H-indol-4-yloxy)ethyl]-4-piperidine]-3-methylbenzothiophene-2-sulphonamide,
N-[[1-[2-(1H-indol-4-yloxy)ethyl]-4-piperidine]methyl]benzenesulphonamide,
N-[[1-[2-(1H-indol-4-yloxy)ethyl]-4-piperidine]methyl]-3-methylbenzenesulphonamide,
3-hydroxy-N-[[1-[2-(1H-indol-4-yloxy)ethyl]-4-piperidine]methyl]benzenesulphonamide,
N-[[1-[2-(1H-indol-4-yloxy)ethyl]-4-piperidine]methyl]naphthalene-1-sulphonamide,
N-[[(1-[2-(1H-indol-4-yloxy)ethyl]-4-piperidine]methyl]naphthalene-2-sulphonamide,
N-[1-[2-(1H-indol-6-yloxy)ethyl]pyrrolidin-3-yl]benzenesulphonamide,
N-[1-[2-(1H-indol-6-yloxy)ethyl]pyrrolidin-3-yl]-3-methylbenzenesulphonamide,
N-[[(1-[2-(1H-indol-6-yloxy)ethyl]-4-piperidine]methyl]benzenesulphonamide,
N-[[1-[2-(1H-indol-6-yloxy)ethyl]-4-piperidine]methyl]naphthalene-1-sulphonamide,
N-[[1-[2-(1H-indol-6-yloxy)ethyl]-4-piperidine]methyl]naphthalene-2-sulphonamide,
N-[1-[3-(5-fluoro-1H-indol-3-yl)propyl]pyrrolidin-3-yl]benzenesulphonamide,
3-fluoro-N-[1-[3-(5-fluoro-1H-indol-3-yl)propyl]pyrrolidin-3-yl]benzenesulphonamide,
4-fluoro-N-[1-[3-(5-fluoro-1H-indol-3-yl)propyl]pyrrolidin-3-yl]benzenesulphonamide,
3-chloro-N-[1-[3-(5-fluoro-1H-indol-3-yl)propyl]pyrrolidin-3-yl]benzenesulphonamide,
4-chloro-N-[1-[3-(5-fluoro-1H-indol-3-yl)propyl]pyrrolidin-3-yl]benzenesulphonamide,
N-[1-[3-(5-fluoro-1H-indol-3-yl)propyl]pyrrolidin-3-yl]-3-methyl-benzenesulphonamide,
N-[1-[3-(5-fluoro-1H-indol-3-yl)propyl]pyrrolidin-3-yl]naphthalene-1-sulphonamide,
N-[1-[3-(5-fluoro-1H-indol-3-yl)propyl]pyrrolidin-3-yl]naphthalene-2-sulphonamide,
N-[1-[3-(5-chloro-1H-indol-3-yl)propyl]pyrrolidin-3-yl]benzenesulphonamide,
N-[1-[3-(5-chloro-1H-indol-3-yl)propyl]pyrrolidin-3-yl]-3-fluoro-benzenesulphonamide,
N-[1-[3-(5-chloro-1H-indol-3-yl)propyl]pyrrolidin-3-yl]-4-fluoro-benzenesulphonamide,
3-chloro-N-[1-[3-(5-chloro-1H-indol-3-yl)propyl]pyrrolidin-3-yl]benzenesulphonamide,
4-chloro-N-[1-[3-(5-chloro-1H-indol-3-yl)propyl]pyrrolidin-3-yl]benzenesulphonamide,
N-[1-[3-(5-chloro-1H-indol-3-yl)propyl]pyrrolidin-3-yl]-3-methyl-benzenesulphonamide,
N-[1-[3-(5-chloro-1H-indol-3-yl)propyl]pyrrolidin-3-yl]naphthalene-2-sulphonamide,
N-[1-[2-(2,3-dihydro-1,4-benzodioxan-8-yloxy)ethyl]azetidin-3-yl]benzene-sulphonamide,
N-[1-[2-(2,3-dihydro-1,4-benzodioxan-8-yloxy)ethyl]azetidin-3-yl]-3-fluoro-benzenesulphonamide,
N-[1-[2-(2,3-dihydro-1,4-benzodioxan-8-yloxy)ethyl]azetidin-3-yl]-4-fluoro-benzenesulphonamide,
3-chloro-N-[1-[2-(2,3-dihydro-1,4-benzodioxan-8-yloxy)ethyl]azetidin-3-yl]benzene-sulphonamide,
4-chloro-N-[1-[2-(2,3-dihydro-1,4-benzodioxan-8-yloxy)ethyl]azetidin-3-yl]benzenesulphonamide,
N-[1-[2-(2,3-dihydro-1,4-benzodioxan-8-yloxy)ethyl]azetidin-3-yl]-3-methyl-benzenesulphonamide,
N-[1-[2-(2,3-dihydro-1,4-benzodioxan-8-yloxy)ethyl]azetidin-3-yl]naphthalene-1-sulphonamide,
N-[1-[2-(2,3-dihydro-1,4-benzodioxan-8-yloxy)ethyl]azetidin-3-yl]naphthalene-2-sulphonamide,
N-[1-[2-(2,3-dihydro-1,4-benzodioxan-8-yloxy)ethyl]pyrrolidin-3-yl]benzene-sulphonamide,
N-[1-[2-(2,3-dihydro-1,4-benzodioxan-8-yloxy)ethyl]pyrrolidin-3-yl]-3-methyl-benzene-sulphonamide,
N-[1-[2-(2,3-dihydro-1,4-benzodioxan-8-yloxy)ethyl]pyrrolidin-3-yl]naphthalene-1-sulphonamide
N-[1-[2-(2,3-dihydro-1,4-benzodioxan-8-yloxy)ethyl]pyrrolidin-3-yl]naphthalene-2-sulphonamide,
N-[1-[2-(2,3-dihydro-1,4-benzodioxan-8-yloxy)ethyl]-4-piperidine]naphthalene-1-sulphonamide,
N-[[1-[2-(2,3-dihydro-1,4-benzodioxan-5-yloxy)ethyl]-4-piperidine]methyl]benzenesulphonamide,
N-[[1-[2-(2,3-dihydro-1,4-benzodioxan-5-yloxy)ethyl]-4-piperidine]methyl]-3-methylbenzenesulphonamide,
N-[[1-[2-(2,3-dihydro-1,4-benzodioxan-5-yloxy)ethyl]-4-piperidine]methyl]-3-hydroxybenzenesulphonamide,
N-[[1-[2-(2,3-dihydro-1,4-benzodioxan-5-yloxy)ethyl]-4-piperidine]methyl]-naphthalene-1-sulphonamide,
N-[[1-[2-(2,3-dihydro-1,4-benzodioxan-5-yloxy)ethyl]-4-piperidine]methyl]-naphthalene-2-sulphonamide,
N-[[1-[2-(2-oxoindolin-4-yl)oxyethyl]pyrrolidin-3-yl]methyl]naphthalene-2-sulphonamide,
N-[1-[2-[(2,2-dimethyl-3H-benzofuran-7-yl)oxy]ethyl]pyrrolidin-3-yl]-3-hydroxy-benzenesulphonamide,
N-[1-[2-[(2,2-dimethyl-3H-benzofuran-7-yl)oxy]ethyl]pyrrolidin-3-yl]naphthalene-2-sulphonamide,
N-[1-[2-[(2,2-dimethyl-3H-benzofuran-7-yl)oxy]ethyl]-4-piperidine]-3-hydroxy-benzene-sulphonamide,
N-[1-[2-[(2,2-dimethyl-3H-benzofuran-7-yl)oxy]ethyl]-4-piperidine]naphthalene-2-sulphonamide,
N-[[1-[2-[(2,2-dimethyl-3H-benzofuran-7-yl)oxy]ethyl]azetidin-3-yl]methyl]-3-hydroxy-benzenesulphonamide,
N-[[1-[2-[(2,2-dimethyl-3H-benzofuran-7-yl)oxy]ethyl]azetidin-3-yl]methyl]-naphthalene-2-sulphonamide,
N-[[1-[2-[(2,2-dimethyl-3H-benzofuran-7-yl)oxy]ethyl]pyrrolidin-3-yl]methyl]-3-hydroxy-benzenesulphonamide,
N-[[1-[2-[(2,2-dimethyl-3H-benzofuran-7-yl)oxy]ethyl]pyrrolidin-3-yl]methyl]-naphthalene-2-sulphonamide,
3-hydroxy-N-[1-[2-(1-naphthyloxy)ethyl]pyrrolidin-3-yl]benzenesulphonamide,
N-[1-[2-(1-naphthyloxy)ethyl]pyrrolidin-3-yl]naphthalene-2-sulphonamide,
N-[1-[2-(2,3-dihydro-1,4-benzodioxan-8-yloxy)ethyl]-4-piperidine]benzene-sulphonamide,
N-[1-[2-(2,3-dihydro-1,4-benzodioxan-8-yloxy)ethyl]-4-piperidine]-3-methyl-benzenesulphonamide,
3-hydroxy-N-[1-[2-(1-naphthyloxy)ethyl]-4-piperidine]benzenesulphonamide,
N-[1-[2-(1-naphthyloxy)ethyl]-4-piperidine]naphthalene-2-sulphonamide,
3-hydroxy-N-[[1-[2-(1-naphthyloxy)ethyl]azetidin-3-yl]methyl]benzenesulphonamide,
N-[[1-[2-(1-naphthyloxy)ethyl]azetidin-3-yl]methyl]naphthalene-2-sulphonamide,
3-hydroxy-N-[[1-[2-(1-naphthyloxy)ethyl]pyrrolidin-3-yl]methyl]benzenesulphonamide,
N-[[1-[2-(1-naphthyloxy)ethyl]pyrrolidin-3-yl]methyl]naphthalene-2-sulphonamide,
N-[1-[2-(1,2-benzothiazol-3-yloxy)ethyl]pyrrolidin-3-yl]-3-hydroxybenzene-sulphonamide,
N-[[1-[3-(6-fluoro-1,2-benzoxazol-3-yl)propyl]azetidin-3-yl]methyl]-1,3-benzodioxole-5-sulphonamide,
N-[[1-[3-(6-fluoro-1,2-benzoxazol-3-yl)propyl]azetidin-3-yl]methyl]benzo-thiophene-2-sulphonamide,
N-[[1-[3-(6-fluoro-1,2-benzoxazol-3-yl)propyl]azetidin-3-yl]methyl]-1,3-benzothiazole-4-sulphonamide,
6-chloro-N-[[1-[3-(6-fluoro-1,2-benzoxazol-3-yl)propyl]azetidin-3-yl]-methyl]-naphthalene-2-sulphonamide,
5-fluoro-N-[[1-[3-(6-fluoro-1,2-benzoxazol-3-yl)propyl]azetidin-3-yl]methyl]-3-methyl-benzothiophene-2-sulphonamide,
5-chloro-N-[[1-[3-(6-fluoro-1,2-benzoxazol-3-yl)propyl]azetidin-3-yl]-methyl]-3-methyl-benzothiophene-2-sulphonamide,
N-[[1-[3-(6-fluoro-1,2-benzoxazol-3-yl)propyl]azetidin-3-yl]methyl]-1,3-benzothiazole-5-sulphonamide,
N-[[1-[3-(6-fluoro-1,2-benzoxazol-3-yl)propyl]azetidin-3-yl]methyl]-naphthalene-1-sulphonamide,
N-[[1-[3-(6-fluoro-1,2-benzoxazol-3-yl)propyl]azetidin-3-yl]methyl]-naphthalene-2-sulphonamide,
N-[[1-[3-(6-fluoro-1,2-benzoxazol-3-yl)propyl]azetidin-3-yl]methyl]-4-phenyl-benzenesulphonamide,
4-chloro-N-[[1-[3-(6-fluoro-1,2-benzoxazol-3-yl)propyl]azetidin-3-yl]methyl]benzenesulphonamide,
3-chloro-N-[[1-[3-(6-fluoro-1,2-benzoxazol-3-yl)propyl]azetidin-3-yl]methyl]benzenesulphonamide,
N-[[1-[3-(6-fluoro-1,2-benzoxazol-3-yl)propyl]azetidin-3-yl]methyl]-4-methyl-benzenesulphonamide,
N-[[1-[3-(6-fluoro-1,2-benzoxazol-3-yl)propyl]azetidin-3-yl]methyl]benzenesulphonamide,
N-[[1-[3-(6-fluoro-1,2-benzoxazol-3-yl)propyl]azetidin-3-yl]methyl]-4-(trifluoromethyl)benzenesulphonamide,
4-tert-butyl-N-[[1-[3-(6-fluoro-1,2-benzoxazol-3-yl)propyl]azetidin-3-yl]methyl]benzenesulphonamide,
N-[[1-[3-(6-fluoro-1,2-benzoxazol-3-yl)propyl]azetidin-3-yl]methyl]-3-methyl-benzenesulphonamide,
N-[[1-[3-(6-fluoro-1,2-benzoxazol-3-yl)propyl]azetidin-3-yl]methyl]-3-methoxy-benzenesulphonamide,
3-fluoro-N-[[1-[3-(6-fluoro-1,2-benzoxazol-3-yl)propyl]azetidin-3-yl]methyl]benzenesulphonamide,
4-cyano-N-[[1-[3-(6-fluoro-1,2-benzoxazol-3-yl)propyl]azetidin-3-yl]methyl]benzenesulphonamide,
3,4-dichloro-N-[[1-[3-(6-fluoro-1,2-benzoxazol-3-yl)propyl]azetidin-3-yl]methyl]benzenesulphonamide,
4-fluoro-N-[[1-[3-(6-fluoro-1,2-benzoxazol-3-yl)propyl]azetidin-3-yl]methyl]benzenesulphonamide,
4-bromo-N-[[1-[3-(6-fluoro-1,2-benzoxazol-3-yl)propyl]azetidin-3-yl]methyl]benzenesulphonamide,
N-[[1-[3-(6-fluoro-1,2-benzoxazol-3-yl)propyl]azetidin-3-yl]methyl]-3-hydroxy-benzenesulphonamide,N-[[1-[3-(6-fluoro-1,2-benzoxazol-3-yl)propyl]azetidin-3-yl]methyl]-1-methyl-indole-5-sulphonamide,
N-[[1-[3-(6-fluoro-1,2-benzoxazol-3-yl)propyl]azetidin-3-yl]methyl]benzofuran-2-sulphonamide,
N-[[1-[3-(6-fluoro-1,2-benzoxazol-3-yl)propyl]azetidin-3-yl]methyl]-1-methyl-indole-4-sulphonamide,
N-[[1-[3-(6-fluoro-1,2-benzoxazol-3-yl)propyl]azetidin-3-yl]methyl]benzothiophene-2-sulphonamide,
N-[[1-[3-(6-fluoro-1,2-benzoxazol-3-yl)propyl]azetidin-3-yl]methyl]thiophene-3-sulphonamide,
5-chloro-N-[[1-[3-(6-fluoro-1,2-benzoxazol-3-yl)propyl]azetidin-3-yl]methyl]thiophene-2-sulphonamide,
3-chloro-4-fluoro-N-[[1-[3-(6-fluoro-1,2-benzoxazol-3-yl)propyl]azetidin-3-yl]methyl]benzenesulphonamide,
N-[[1-[3-(6-fluoro-1,2-benzoxazol-3-yl)propyl]azetidin-3-yl]methyl]-4-propyl-benzenesulphonamide,
3,4-difluoro-N-[[1-[3-(6-fluoro-1,2-benzoxazol-3-yl)propyl]azetidin-3-yl]methyl]benzenesulphonamide,
N-[[1-[3-(6-fluoro-1,2-benzoxazol-3-yl)propyl]azetidin-3-yl]methyl]-4-(trifluoromethoxy)benzenesulphonamide,
N-[[1-[3-(5-fluoro-1H-indol-3-yl)propyl]azetidin-3-yl]methyl]naphthalene-2-sulphonamide,
N-[[1-[3-(5-chloro-1H-indol-3-yl)propyl]azetidin-3-yl]methyl]naphthalene-2-sulphonamide,
N-[[1-[2-(1,2-benzothiazol-3-yloxy)ethyl]azetidin-3-yl]methyl]-naphthalene-2-sulphonamide,
N-[[1-[2-(1-naphthyloxy)ethyl]azetidin-3-yl]methyl]benzothiophene-2-sulphonamide,
6-chloro-N-[[1-[2-(1-naphthyloxy)ethyl]azetidin-3-yl]methyl]-benzothiophene-2-sulphonamide,
6-chloro-N-[[1-[2-(1-naphthyloxy)ethyl]azetidin-3-yl]methyl]naphthalene-2-sulphonamide,
5-fluoro-3-methyl-N-[[1-[2-(1-naphthyloxy)ethyl]azetidin-3-yl]methyl]benzothiophene-2-sulphonamide,
5-chloro-3-methyl-N-[[1-[2-(1-naphthyloxy)ethyl]azetidin-3-yl]methyl]benzothiophene-2-sulphonamide,
N-[[1-[2-(1-naphthyloxy)ethyl]azetidin-3-yl]methyl]naphthalene-1-sulphonamide,
1-methyl-N-[[1-[2-(1-naphthyloxy)ethyl]azetidin-3-yl]methyl]indole-5-sulphonamide,
1-methyl-N-[[1-[2-(1-naphthyloxy)ethyl]azetidin-3-yl]methyl]indole-4-sulphonamide,
N-[[1-[2-(1-naphthyloxy)ethyl]azetidin-3-yl]methyl]benzothiophene-3-sulphonamide,
3-chloro-4-fluoro-N-[[1-[2-(1-naphthyloxy)ethyl]azetidin-3-yl]methyl]benzenesulphonamide,
3,4-difluoro-N-[[1-[2-(1-naphthyloxy)ethyl]azetidin-3-yl]methyl]benzenesulphonamide,
6-chloro-N-[[1-[2-(2,3-dihydro-1,4-benzodioxin-5-yloxy)ethyl]azetidin-3-yl]methyl]naphthalene-2-sulphonamide,
5-chloro-N-[[1-[2-(2,3-dihydro-1,4-benzodioxin-5-yloxy)ethyl]azetidin-3-yl]methyl]-3-methylbenzothiophene-2-sulphonamide,
N-[[1-[2-(2,3-dihydro-1,4-benzodioxin-5-yloxy)ethyl]azetidin-3-yl]methyl]-naphthalene-1-sulphonamide,
N-[[1-[2-(2,3-dihydro-1,4-benzodioxin-5-yloxy)ethyl]azetidin-3-yl]methyl]-naphthalene-2-sulphonamide,
N-[[1-[2-(2,3-dihydro-1,4-benzodioxin-5-yloxy)ethyl]azetidin-3-yl]methyl]-4-phenyl-benzenesulphonamide,
4-chloro-N-[[1-[2-(2,3-dihydro-1,4-benzodioxin-5-yloxy)ethyl]azetidin-3-yl]methyl]benzenesulphonamide,
N-[[1-[2-(2,3-dihydro-1,4-benzodioxin-5-yloxy)ethyl]azetidin-3-yl]methyl]-4-(trifluoromethyl)benzenesulphonamide,
4-tert-butyl-N-[[1-[2-(2,3-dihydro-1,4-benzodioxin-5-yloxy)ethyl]azetidin-3-yl]methyl]benzenesulphonamide,
N-[[1-[2-(2,3-dihydro-1,4-benzodioxin-5-yloxy)ethyl]azetidin-3-yl]methyl]-4-fluoro-benzenesulphonamide
3,4-dichloro-N-[[1-[2-(2,3-dihydro-1,4-benzodioxin-5-yloxy)ethyl]azetidin-3-yl]methyl]benzenesulphonamide,
N-[[1-[2-(2,3-dihydro-1,4-benzodioxin-5-yloxy)ethyl]azetidin-3-yl]methyl]-thiophene-2-sulphonamide,
4-bromo-N-[[1-[2-(2,3-dihydro-1,4-benzodioxin-5-yloxy)ethyl]azetidin-3-yl]methyl]benzenesulphonamide,
N-[[1-[2-(2,3-dihydro-1,4-benzodioxin-5-yloxy)ethyl]azetidin-3-yl]methyl]benzofuran-2-sulphonamide,
N-[[1-[2-(2,3-dihydro-1,4-benzodioxin-5-yloxy)ethyl]azetidin-3-yl]methyl]-1-methyl-indole-5-sulphonamide,
N-[[1-[2-(2,3-dihydro-1,4-benzodioxin-5-yloxy)ethyl]azetidin-3-yl]methyl]-1-methyl-indole-4-sulphonamide,
N-[[1-[2-(2,3-dihydro-1,4-benzodioxin-5-yloxy)ethyl]azetidin-3-yl]methyl]-2-oxo-indoline-5-sulphonamide,
N-[[1-[2-(2,3-dihydro-1,4-benzodioxin-5-yloxy)ethyl]azetidin-3-yl]methyl]benzothiophene-3-sulphonamide,
N-[[1-[2-(2,3-dihydro-1,4-benzodioxin-5-yloxy)ethyl]azetidin-3-yl]methyl]-thiophene-3-sulphonamide,
5-chloro-N-[[1-[2-(2,3-dihydro-1,4-benzodioxin-5-yloxy)ethyl]azetidin-3-yl]methyl]thiophene-2-sulphonamide,
N-[[1-[2-(2,3-dihydro-1,4-benzodioxin-5-yloxy)ethyl]azetidin-3-yl]methyl]-4-iodo-benzenesulphonamide,
N-[1-[3-(6-fluoro-1,2-benzoxazol-3-yl)propyl]pyrrolidin-3-yl]-1,3-benzo-dioxole-5-sulphonamide,
N-[1-[3-(6-fluoro-1,2-benzoxazol-3-yl)propyl]pyrrolidin-3-yl]-4-phenylbenzenesulphonamide,
N-[(3R)-1-[3-(6-fluoro-1,2-benzoxazol-3-yl)propyl]pyrrolidin-3-yl]benzothiophene-2-sulphonamide,
N-[(3S)-1-[3-CE-fluoro-1,2-benzoxazol-3-yl)propyl]pyrrolidin-3-yl]benzothiophene-2-sulphonamide,
6-chloro-N-[(3R)-1-[3-(6-fluoro-1,2-benzoxazol-3-yl)propyl]pyrrolidin-3-yl]benzothiophene-2-sulphonamide,
6-chloro-N-[(3S)-1-[3-(6-fluoro-1,2-benzoxazol-3-yl)propyl]pyrrolidin-3-yl]benzothiophene-2-sulphonamide,
6-chloro-N-[(3R)-1-[3-(6-fluoro-1,2-benzoxazol-3-yl)propyl]pyrrolidin-3-yl]naphthalene-2-sulphonamide,
6-chloro-N-[(3S)-1-[3-(6-fluoro-1,2-benzoxazol-3-yl)propyl]pyrrolidin-3-yl]naphthalene-2-sulphonamide,
5-fluoro-N-[(3R)-1-[3-(6-fluoro-1,2-benzoxazol-3-yl)propyl]pyrrolidin-3-yl]-3-methyl-benzothiophene-2-sulphonamide,
5-fluoro-N-[(3S)-1-[3-(6-fluoro-1,2-benzoxazol-3-yl)propyl]pyrrolidin-3-yl]-3-methyl-benzothiophene-2-sulphonamide,
5-chloro-N-[(3R)-1-[3-(6-fluoro-1,2-benzoxazol-3-yl)propyl]pyrrolidin-3-yl]-3-methyl-benzothiophene-2-sulphonamide,
5-chloro-N-[(3S)-1-[3-(6-fluoro-1,2-benzoxazol-3-yl)propyl]pyrrolidin-3-yl]-3-methyl-benzothiophene-2-sulphonamide,
N-[(3R)-1-[3-(6-fluoro-1,2-benzoxazol-3-yl)propyl]pyrrolidin-3-yl]naphthalene-2-sulphonamide,
N-[(3S)-1-[3-(6-fluoro-1,2-benzoxazol-3-yl)propyl]pyrrolidin-3-yl]-naphthalene-2-sulphonamide,
4-chloro-N-[1-[3-(6-fluoro-1,2-benzoxazol-3-yl)propyl]pyrrolidin-3-yl]benzenesulphonamide,
N-[1-[3-(6-fluoro-1,2-benzoxazol-3-yl)propyl]pyrrolidin-3-yl]-4-methylbenzenesulphonamide,
4-cyano-N-[1-[3-(6-fluoro-1,2-benzoxazol-3-yl)propyl]pyrrolidin-3-yl]benzenesulphonamide
3,4-dichloro-N-[1-[3-(6-fluoro-1,2-benzoxazol-3-yl)propyl]pyrrolidin-3-yl]benzenesulphonamide,
N-[1-[3-(6-fluoro-1,2-benzoxazol-3-yl)propyl]pyrrolidin-3-yl]thiophene-2-sulphonamide,
N-[1-[3-(6-fluoro-1,2-benzoxazol-3-yl)propyl]pyrrolidin-3-yl]-4-methoxybenzenesulphonamide,
N-[(3R)-1-[3-(6-fluoro-1,2-benzoxazol-3-yl)propyl]pyrrolidin-3-yl]benzo-furan-2-sulphonamide,
N-[(3S)-1-[3-(6-fluoro-1,2-benzoxazol-3-yl)propyl]pyrrolidin-3-yl]benzo-furan-2-sulphonamide,
N-[1-[3-(6-fluoro-1,2-benzoxazol-3-yl)propyl]pyrrolidin-3-yl]benzofuran-2-sulphonamide, N-[1-[3-(6-fluoro-1,2-benzoxazol-3-yl)propyl]pyrrolidin-3-yl]-1-methyl-imidazole-4-sulphonamide,
N-[1-[3-(6-fluoro-1,2-benzoxazol-3-yl)propyl]pyrrolidin-3-yl]-1-methyl-indole-5-sulphonamide,
N-[1-[3-(6-fluoro-1,2-benzoxazol-3-yl)propyl]pyrrolidin-3-yl]-1-methyl-indole-4-sulphonamide,
N-[1-[3-(6-fluoro-1,2-benzoxazol-3-yl)propyl]pyrrolidin-3-yl]-2-oxo-indoline-5-sulphonamide,
N-[1-[3-(6-fluoro-1,2-benzoxazol-3-yl)propyl]pyrrolidin-3-yl]-2,5-dimethyl-thiophene-3-sulphonamide,
N-[(3R)-1-[3-(6-fluoro-1,2-benzoxazol-3-yl)propyl]pyrrolidin-3-yl]-2,5-dimethyl-thiophene-3-sulphonamide,
N-[(3S)-1-[3-(6-fluoro-1,2-benzoxazol-3-yl)propyl]pyrrolidin-3-yl]-2,5-dimethyl-thiophene-3-sulphonamide,
N-[(3R)-1-[3-(6-fluoro-1,2-benzoxazol-3-yl)propyl]pyrrolidin-3-yl]-benzothiophene-3-sulphonamide
N-[(3S)-1-[3-(6-fluoro-1,2-benzoxazol-3-yl)propyl]pyrrolidin-3-yl]-benzothiophene-3-sulphonamide,
N-[1-[3-(6-fluoro-1,2-benzoxazol-3-yl)propyl]pyrrolidin-3-yl]-5-methylbenzothiophene-2-sulphonamide,
N-[1-[3-(6-fluoro-1,2-benzoxazol-3-yl)propyl]pyrrolidin-3-yl]-6-methoxy-naphthalene-2-sulphonamide,
N-[(3R)-1-[3-(6-fluoro-1,2-benzoxazol-3-yl)propyl]pyrrolidin-3-yl]-5-methylbenzothiophene-2-sulphonamide,
N-[(3S)-1-[3-(6-fluoro-1,2-benzoxazol-3-yl)propyl]pyrrolidin-3-yl]-5-methylbenzothiophene-2-sulphonamide,
N-[(3R)-1-[3-(6-fluoro-1,2-benzoxazol-3-yl)propyl]pyrrolidin-3-yl]-6-methoxy-naphthalene-2-sulphonamide,
N-[(3S)-1-[3-(6-fluoro-1,2-benzoxazol-3-yl)propyl]pyrrolidin-3-yl]-6-methoxy-naphthalene-2-sulphonamide,
7-chloro-N-[(3R)-1-[3-(6-fluoro-1,2-benzoxazol-3-yl)propyl]pyrrolidin-3-yl]naphthalene-2-sulphonamide,
7-chloro-N-[(3S)-1-[3-(6-fluoro-1,2-benzoxazol-3-yl)propyl]pyrrolidin-3-yl]naphthalene-2-sulphonamide,
6-fluoro-N-[1-[3-(6-fluoro-1,2-benzoxazol-3-yl)propyl]pyrrolidin-3-yl]benzothiophene-2-sulphonamide,
N-[[1-[3-(6-fluoro-1,2-benzoxazol-3-yl)propyl]pyrrolidin-3-yl]methyl]-1,3-benzodioxole-5-sulphonamide,
N-[[1-[3-(6-fluoro-1,2-benzoxazol-3-yl)propyl]pyrrolidin-3-yl]methyl]-1,3-benzothiazole-4-sulphonamide,
6-chloro-N-[[1-[3-(6-fluoro-1,2-benzoxazol-3-yl)propyl]pyrrolidin-3-yl]methyl]naphthalene-2-sulphonamide,
5-chloro-N-[[1-[3-(6-fluoro-1,2-benzoxazol-3-yl)propyl]pyrrolidin-3-yl]methyl]-3-methylbenzothiophene-2-sulphonamide,
N-[[1-[3-(6-fluoro-1,2-benzoxazol-3-yl)propyl]pyrrolidin-3-yl]methyl]naphthalene-1-sulphonamide,
N-[[1-[3-(6-fluoro-1,2-benzoxazol-3-yl)propyl]pyrrolidin-3-yl]methyl]-naphthalene-2-sulphonamide,
N-[[1-[3-(6-fluoro-1,2-benzoxazol-3-yl)propyl]pyrrolidin-3-yl]methyl]-4-phenyl-benzenesulphonamide,
4-chloro-N-[[1-[3-(6-fluoro-1,2-benzoxazol-3-yl)propyl]pyrrolidin-3-yl]methyl]benzenesulphonamide,
3-chloro-N-[[1-[3-(6-fluoro-1,2-benzoxazol-3-yl)propyl]pyrrolidin-3-yl]methyl]benzenesulphonamide,
N-[[1-[3-(6-fluoro-1,2-benzoxazol-3-yl)propyl]pyrrolidin-3-yl]methyl]-4-methyl-benzenesulphonamide,
N-[[1-[3-(6-fluoro-1,2-benzoxazol-3-yl)propyl]pyrrolidin-3-yl]methyl]benzenesulphonamide,
N-[[1-[3-(6-fluoro-1,2-benzoxazol-3-yl)propyl]pyrrolidin-3-yl]methyl]-4-(trifluoromethyl)benzenesulphonamide,
N-[[1-[3-(6-fluoro-1,2-benzoxazol-3-yl)propyl]pyrrolidin-3-yl]methyl]-3-(trifluoromethyl)benzenesulphonamide,
4-tert-butyl-N-[[1-[3-(6-fluoro-1,2-benzoxazol-3-yl)propyl]pyrrolidin-3-yl]methyl]benzenesulphonamide,
3-tert-butyl-N-[[1-[3-(6-fluoro-1,2-benzoxazol-3-yl)propyl]pyrrolidin-3-yl]methyl]benzenesulphonamide,
N-[[1-[3-(6-fluoro-1,2-benzoxazol-3-yl)propyl]pyrrolidin-3-yl]methyl]-3-methoxy-benzenesulphonamide,
4-cyano-N-[[1-[3-(6-fluoro-1,2-benzoxazol-3-yl)propyl]pyrrolidin-3-yl]methyl]benzenesulphonamide,
4-fluoro-N-[[1-[3-(6-fluoro-1,2-benzoxazol-3-yl)propyl]pyrrolidin-3-yl]methyl]benzenesulphonamide,
3,4-dichloro-N-[[1-[3-(6-fluoro-1,2-benzoxazol-3-yl)propyl]pyrrolidin-3-yl]methyl]benzenesulphonamide,
N-[[1-[3-(6-fluoro-1,2-benzoxazol-3-yl)propyl]pyrrolidin-3-yl]methyl]-3-hydroxy-benzenesulphonamide,
N-[[1-[3-(6-fluoro-1,2-benzoxazol-3-yl)propyl]pyrrolidin-3-yl]methyl]-4-methoxy-benzenesulphonamide,
N-[[1-[3-(6-fluoro-1,2-benzoxazol-3-yl)propyl]pyrrolidin-3-yl]methyl]-2,3-dihydrobenzofuran-5-sulphonamide,
N-[[1-[3-(6-fluoro-1,2-benzoxazol-3-yl)propyl]pyrrolidin-3-yl]methyl]-benzofuran-2-sulphonamide,
N-[[1-[3-(6-fluoro-1,2-benzoxazol-3-yl)propyl]pyrrolidin-3-yl]methyl]-1-methyl-indole-5-sulphonamide,
N-[[1-[3-(6-fluoro-1,2-benzoxazol-3-yl)propyl]pyrrolidin-3-yl]methyl]-1-methyl-indole-4-sulphonamide,
N-[[1-[3-(6-fluoro-1,2-benzoxazol-3-yl)propyl]pyrrolidin-3-yl]methyl]-2-oxo-indoline-5-sulphonamide,
N-[[1-[3-(6-fluoro-1,2-benzoxazol-3-yl)propyl]pyrrolidin-3-yl]methyl]benzothiophene-3-sulphonamide,
N-[[1-[3-(6-fluoro-1,2-benzoxazol-3-yl)propyl]pyrrolidin-3-yl]methyl]-2,5-dimethyl-thiophene-3-sulphonamide,
3-chloro-4-fluoro-N-[[1-[3-(6-fluoro-1,2-benzoxazol-3-yl)propyl]pyrrolidin-3-yl]methyl]benzenesulphonamide,
N-[[1-[3-(6-fluoro-1,2-benzoxazol-3-yl)propyl]pyrrolidin-3-yl]methyl]-4-propyl-benzenesulphonamide,
3,4-difluoro-N-[[1-[3-(6-fluoro-1,2-benzoxazol-3-yl)propyl]pyrrolidin-3-yl]methyl]benzenesulphonamide,
N-[[1-[3-(6-fluoro-1,2-benzoxazol-3-yl)propyl]pyrrolidin-3-yl]methyl]-4-(trifluoromethoxy)benzenesulphonamide,
N-[[1-[3-(5-fluoro-1H-indol-3-yl)propyl]pyrrolidin-3-yl]methyl]-naphthalene-2-sulphonamide,
N-[[1-[2-(1-naphthyloxy)ethyl]pyrrolidin-3-yl]methyl]benzothiophene-2-sulphonamide,
6-chloro-N-[[1-[2-(1-naphthyloxy)ethyl]pyrrolidin-3-yl]methyl]-benzothiophene-2-sulphonamide,
6-chloro-N-[[1-[2-(1-naphthyloxy)ethyl]pyrrolidin-3-yl]methyl]naphthalene-2-sulphonamide,
5-fluoro-3-methyl-N-[[1-[2-(1-naphthyloxy)ethyl]pyrrolidin-3-yl]methyl]benzothiophene-2-sulphonamide,
5-chloro-3-methyl-N-[[1-[2-(1-naphthyloxy)ethyl]pyrrolidin-3-yl]methyl]benzothiophene-2-sulphonamide,
N-[[1-[2-(1-naphthyloxy)ethyl]pyrrolidin-3-yl]methyl]naphthalene-1-sulphonamide,
1-methyl-N-[[1-[2-(1-naphthyloxy)ethyl]pyrrolidin-3-yl]methyl]indole-5-sulphonamide,
1-methyl-N-[[1-[2-(1-naphthyloxy)ethyl]pyrrolidin-3-yl]methyl]indole-4-sulphonamide,
N-[[1-[2-(1-naphthyloxy)ethyl]pyrrolidin-3-yl]methyl]benzothiophene-3-sulphonamide,
3-chloro-4-fluoro-N-[[1-[2-(1-naphthyloxy)ethyl]pyrrolidin-3-yl]methyl-benzene-sulphonamide,
3,4-difluoro-N-[[1-[2-(1-naphthyloxy)ethyl]pyrrolidin-3-yl]methyl]-benzenesulphonamide,
N-[[1-[2-(2,3-dihydro-1,4-benzodioxin-5-yloxy)ethyl]pyrrolidin-3-yl]methyl]-1,3-benzodioxole-5-sulphonamide,
N-[[1-[2-(2,3-dihydro-1,4-benzodioxin-5-yloxy)ethyl]pyrrolidin-3-yl]methyl]benzothiophene-2-sulphonamide,
N-[[1-[2-(2,3-dihydro-1,4-benzodioxin-5-yloxy)ethyl]pyrrolidin-3-yl]methyl]-1,3-benzothiazole-4-sulphonamide,
6-chloro-N-[[1-[2-(2,3-dihydro-1,4-benzodioxin-5-yloxy)ethyl]pyrrolidin-3-yl]methyl]naphthalene-2-sulphonamide,
5-chloro-N-[[1-[2-(2,3-dihydro-1,4-benzodioxin-5-yloxy)ethyl]pyrrolidin-3-yl]methyl]-3-methyl-benzothiophene-2-sulphonamide,
N-[[1-[2-(2,3-dihydro-1,4-benzodioxin-5-yloxy)ethyl]pyrrolidin-3-yl]methyl]thiazole-2-sulphonamide,
N-[[1-[2-(2,3-dihydro-1,4-benzodioxin-5-yloxy)ethyl]pyrrolidin-3-yl]methyl]-1,3-benzothiazole-5-sulphonamide,
N-[[1-[2-(2,3-dihydro-1,4-benzodioxin-5-yloxy)ethyl]pyrrolidin-3-yl]methyl]naphthalene-1-sulphonamide,
N-[[1-[2-(2,3-dihydro-1,4-benzodioxin-5-yloxy)ethyl]pyrrolidin-3-yl]methyl]naphthalene-2-sulphonamide,
N-[[1-[2-(2,3-dihydro-1,4-benzodioxin-5-yloxy)ethyl]pyrrolidin-3-yl]-methyl]-4-phenyl-benzenesulphonamide,
4-chloro-N-[[1-[2-(2,3-dihydro-1,4-benzodioxin-5-yloxy)ethyl]pyrrolidin-3-yl]methyl]benzenesulphonamide,
3-chloro-N-[[1-[2-(2,3-dihydro-1,4-benzodioxin-5-yloxy)ethyl]pyrrolidin-3-yl]methyl]benzenesulphonamide,
N-[[1-[2-(2,3-dihydro-1,4-benzodioxin-5-yloxy)ethyl]pyrrolidin-3-yl]methyl]-4-methyl-benzenesulphonamide,
N-[[1-[2-(2,3-dihydro-1,4-benzodioxin-5-yloxy)ethyl]pyrrolidin-3-yl]methyl]benzenesulphonamide,
N-[[1-[2-(2,3-dihydro-1,4-benzodioxin-5-yloxy)ethyl]pyrrolidin-3-yl]methyl]-4-(trifluoro-methyl)benzenesulphonamide,
N-[[1-[2-(2,3-dihydro-1,4-benzodioxin-5-yloxy)ethyl]pyrrolidin-3-yl]methyl]-3-(trifluoromethyl)benzenesulphonamide,
4-tert-butyl-N-[[1-[2-(2,3-dihydro-1,4-benzodioxin-5-yloxy)ethyl]pyrrolidin-3-yl]methyl]benzenesulphonamide,
N-[[1-[2-(2,3-dihydro-1,4-benzodioxin-5-yloxy)ethyl]pyrrolidin-3-yl]methyl]-3-methyl-benzenesulphonamide,
N-[[1-[2-(2,3-dihydro-1,4-benzodioxin-5-yloxy)ethyl]pyrrolidin-3-yl]-methyl]-3-methoxybenzenesulphonamide,
N-[[1-[2-(2,3-dihydro-1,4-benzodioxin-5-yloxy)ethyl]pyrrolidin-3-yl]-methyl]-3-fluoro-benzenesulphonamide,
4-cyano-N-[[1-[2-(2,3-dihydro-1,4-benzodioxin-5-yloxy)ethyl]pyrrolidin-3-yl]methyl]benzenesulphonamide,
N-[[1-[2-(2,3-dihydro-1,4-benzodioxin-5-yloxy)ethyl]pyrrolidin-3-yl]methyl]-4-fluoro-benzenesulphonamide,
3,4-dichloro-N-[[1-[2-(2,3-dihydro-1,4-benzodioxin-5-yloxy)ethyl]pyrrolidin-3-yl]methyl]benzenesulphonamide,
N-[[1-[2-(2,3-dihydro-1,4-benzodioxin-5-yloxy)ethyl]pyrrolidin-3-yl]methyl]thiophene-2-sulphonamide,
N-[[1-[2-(2,3-dihydro-1,4-benzodioxin-5-yloxy)ethyl]pyrrolidin-3-yl]methyl]-3-hydroxybenzenesulphonamide,
N-[[1-[2-(2,3-dihydro-1,4-benzodioxin-5-yloxy)ethyl]pyrrolidin-3-yl]methyl]-4-methoxybenzenesulphonamide,
4-bromo-N-[[1-[2-(2,3-dihydro-1,4-benzodioxin-5-yloxy)ethyl]pyrrolidin-3-yl]methyl]benzenesulphonamide,
N-[[1-[2-(2,3-dihydro-1,4-benzodioxin-5-yloxy)ethyl]pyrrolidin-3-yl]methyl]-2,3-dihydrobenzofuran-5-sulphonamide,
N-[[1-[2-(2,3-dihydro-1,4-benzodioxin-5-yloxy)ethyl]pyrrolidin-3-yl]methyl]benzofuran-2-sulphonamide,
N-[[1-[2-(2,3-dihydro-1,4-benzodioxin-5-yloxy)ethyl]pyrrolidin-3-yl]methyl]-1-methyl-indole-5-sulphonamide,
N-[[1-[2-(2,3-dihydro-1,4-benzodioxin-5-yloxy)ethyl]pyrrolidin-3-yl]methyl]-1-methyl-indole-4-sulphonamide,
N-[[1-[2-(2,3-dihydro-1,4-benzodioxin-5-yloxy)ethyl]pyrrolidin-3-yl]methyl]-2-oxo-indoline-5-sulphonamide,
N-[[1-[2-(2,3-dihydro-1,4-benzodioxin-5-yloxy)ethyl]pyrrolidin-3-yl]methyl]benzothiophene-3-sulphonamide,
N-[[1-[2-(2,3-dihydro-1,4-benzodioxin-5-yloxy)ethyl]pyrrolidin-3-yl]methyl]-2,5-dimethyl-thiophene-3-sulphonamide,
N-[[1-[2-(2,3-dihydro-1,4-benzodioxin-5-yloxy)ethyl]pyrrolidin-3-yl]methyl]thiophene-3-sulphonamide,
N-[[(1-[2-(2,3-dihydro-1,4-benzodioxin-5-yloxy)ethyl]pyrrolidin-3-yl]methyl]-5-isoxazol-5-yl-thiophene-2-sulphonamide,
3-cyano-N-[[1-[2-(2,3-dihydro-1,4-benzodioxin-5-yloxy)ethyl]pyrrolidin-3-yl]methyl]benzenesulphonamide,
5-chloro-N-[[1-[2-(2,3-dihydro-1,4-benzodioxin-5-yloxy)ethyl]pyrrolidin-3-yl]methyl]thiophene-2-sulphonamide,
3-chloro-N-[[1-[2-(2,3-dihydro-1,4-benzodioxin-5-yloxy)ethyl]pyrrolidin-3-yl]methyl]-4-fluoro-benzenesulphonamide,
N-[[1-[2-(2,3-dihydro-1,4-benzodioxin-5-yloxy)ethyl]pyrrolidin-3-yl]methyl]-4-propyl-benzenesulphonamide,
N-[[1-[2-(2,3-dihydro-1,4-benzodioxin-5-yloxy)ethyl]pyrrolidin-3-yl]methyl]-3,4-difluoro-benzenesulphonamide,
N-[[1-[2-(2,3-dihydro-1,4-benzodioxin-5-yloxy)ethyl]pyrrolidin-3-yl]methyl]-4-(trifluoromethoxy)benzenesulphonamide,
N-[[1-[2-(2,3-dihydro-1,4-benzodioxin-5-yloxy)ethyl]pyrrolidin-3-yl]methyl]-4-iodo-benzenesulphonamide,
3-bromo-N-[[1-[2-(2,3-dihydro-1,4-benzodioxin-5-yloxy)ethyl]pyrrolidin-3-yl]methyl]benzenesulphonamide,
N-[[1-[2-(2,3-dihydro-1,4-benzodioxin-5-yloxy)ethyl]pyrrolidin-3-yl]methyl]-5-methyl-isoxazole-4-sulphonamide,
N-[1-[3-(6-fluoro-1,2-benzoxazol-3-yl)propyl]-4-piperidyl]benzothiophene-2-sulphonamide
6-chloro-N-[1-[3-(6-fluoro-1,2-benzoxazol-3-yl)propyl]-4-piperidyl]naphthalene-2-sulphonamide,
5-chloro-N-[1-[3-(6-fluoro-1,2-benzoxazol-3-yl)propyl]-4-piperidyl]-3-methyl-benzothiophene-2-sulphonamide,
N-[1-[3-(6-fluoro-1,2-benzoxazol-3-yl)propyl]-4-piperidyl]-4-phenyl-benzenesulphonamide,
4-tert-butyl-N-[1-[3-(6-fluoro-1,2-benzoxazol-3-yl)propyl]-4-piperidyl]-benzenesulphonamide,
N-[1-[3-(6-fluoro-1,2-benzoxazol-3-yl)propyl]-4-piperidyl]-1-methyl-indole-4-sulphonamide,
N-[1-[3-(6-fluoro-1,2-benzoxazol-3-yl)propyl]-4-piperidyl]benzothiophene-3-sulphonamide,
N-[1-[3-(5-chloro-1H-indol-3-yl)propyl]pyrrolidin-3-yl]benzothiophene-2-sulphonamide,
6-chloro-N-[1-[3-(5-chloro-1H-indol-3-yl)propyl]pyrrolidin-3-yl]benzothiophene-2-sulphonamide,
6-chloro-N-[1-[3-(5-chloro-1H-indol-3-yl)propyl]pyrrolidin-3-yl]naphthalene-2-sulphonamide,
N-[1-[3-(5-chloro-1H-indol-3-yl)propyl]pyrrolidin-3-yl]-5-fluoro-3-methyl-benzothiophene-2-sulphonamide,
5-chloro-N-[1-[3-(5-chloro-1H-indol-3-yl)propyl]pyrrolidin-3-yl]-3-methyl-benzothiophene-2-sulphonamide,
3,4-dichloro-N-[1-[3-(5-chloro-1H-indol-3-yl)propyl]pyrrolidin-3-yl]benzenesulphonamide,
N-[1-[3-(5-chloro-1H-indol-3-yl)propyl]pyrrolidin-3-yl]thiophene-2-sulphonamide,
N-[1-[3-(5-chloro-1H-indol-3-yl)propyl]pyrrolidin-3-yl]-1-methyl-indole-5-sulphonamide,
N-[1-[3-(5-chloro-1H-indol-3-yl)propyl]pyrrolidin-3-yl]-1-methyl-indole-4-sulphonamide,
N-[1-[3-(5-chloro-1H-indol-3-yl)propyl]pyrrolidin-3-yl]benzothiophene-3-sulphonamide,
N-[1-[3-(5-chloro-1H-indol-3-yl)propyl]pyrrolidin-3-yl]naphthalene-1-sulphonamide,
3-chloro-N-[1-[3-(5-chloro-1H-indol-3-yl)propyl]pyrrolidin-3-yl]-4-fluorobenzenesulphonamide,
N-[1-[3-(5-chloro-1H-indol-3-yl)propyl]pyrrolidin-3-yl]-3,4-difluorobenzenesulphonamide,
N-[1-[3-(5-fluoro-1H-indol-3-yl)propyl]pyrrolidin-3-yl]benzothiophene-2-sulphonamide,
6-chloro-N-[1-[3-(5-fluoro-1H-indol-3-yl)propyl]pyrrolidin-3-yl]benzothiophene-2-sulphonamide,
6-chloro-N-[1-[3-(5-fluoro-1H-indol-3-yl)propyl]pyrrolidin-3-yl]naphthalene-2-sulphonamide,
5-fluoro-N-[1-[3-(5-fluoro-1H-indol-3-yl)propyl]pyrrolidin-3-yl]-3-methyl-benzothiophene-2-sulphonamide,
5-chloro-N-[1-[3-(5-fluoro-1H-indol-3-yl)propyl]pyrrolidin-3-yl]-3-methylbenzothiophene-2-sulphonamide,
3,4-dichloro-N-[1-[3-(5-fluoro-1H-indol-3-yl)propyl]pyrrolidin-3-yl]benzenesulphonamide,
N-[1-(3-(5-fluoro-1H-indol-3-yl)propyl]pyrrolidin-3-yl)thiophene-2-sulphonamide,
N-[1-[3-(5-fluoro-1H-indol-3-yl)propyl]pyrrolidin-3-yl]-1-methyl-indole-5-sulphonamide,
N-[1-[3-(5-fluoro-1H-indol-3-yl)propyl]pyrrolidin-3-yl]-1-methyl-indole-4-sulphonamide,
N-[1-[3-(5-fluoro-1H-indol-3-yl)propyl]pyrrolidin-3-yl]benzothiophene-3-sulphonamide,
3-chloro-4-fluoro-N-[1-[3-(5-fluoro-1H-indol-3-yl)propyl]pyrrolidin-3-yl]benzenesulphonamide,
3,4-difluoro-N-[1-[3-(5-fluoro-1H-indol-3-yl)propyl]pyrrolidin-3-yl]benzenesulphonamide,
and pharmaceutically acceptable salts and solvates thereof.

19. (canceled)

20. A pharmaceutical composition comprising a compound of formula (I) as defined in claim 1 as an active ingredient in combination with pharmaceutically acceptable carrier(s) and/or excipient(s).

21-22. (canceled)

23. A method of treatment and/or prevention of disorders of the central nervous system related to serotoninergic and dopaminergic transmission in mammals, comprising administration of the pharmaceutically effective amount of a compound of formula (I) as defined in claim 1 wherein the disorder of the central nervous system is selected from the group consisting of schizophrenia; schizoaffective disorders; schizophreniform disorders; delusional syndromes and other psychotic conditions related and not related to taking psychoactive substances; affective disorder; bipolar disorder; mania; depression; anxiety disorders of various etiology; stress reactions; consciousness disorders; coma; delirium of alcoholic or other etiology; aggression; psychomotor agitation and other conduct disorders; sleep disorders of various etiology; withdrawal syndromes of various etiology; addiction; pain syndromes of various etioloqy; intoxication with psychoactive substances; cerebral circulatory disorders of various etiology psychosomatic disorders of various etiology; conversion disorders; dissociative disorders; urination disorders; autism and other developmental disorders, including nocturia, stuttering, tics; cognitive disorders of various types, including Alzheimer's disease; and psychopathological symptoms and neurological disorders in the course of other diseases of the central and peripheral nervous systems.
